(19) United States
(12) Patent Application Publication
(10) Pub. No.: US 2011/0104680 A1
(43) Pub. Date: May 5, 2011

(54) RECURRENT GENE FUSIONS IN LUNG CANCER

(75) Inventors: Arul M. Chinnaiyan, Plymouth, MI (US); Xiaosong Wang, Houston, TX (US)

(73) Assignee: THE REGENTS OF THE UNIVERSITY OF MICHIGAN, Ann Arbor, MI (US)

(21) Appl. No.: 12/893,801

(22) Filed: Sep. 29, 2010

Related U.S. Application Data
(60) Provisional application No. 61/249,089, filed on Oct. 6, 2009.

(51) Int. Cl. C12Q 1/68; C07H 21/00; C07K 16/18; G01N 33/53; G01N 33/68

(52) U.S. Cl. 435/6; 536/24.31; 536/24.33; 530/389.1; 436/94; 435/7.1; 436/86

(57) ABSTRACT

The present invention relates to compositions and methods for cancer diagnosis, research and therapy, including but not limited to, cancer markers. In particular, the present invention relates to recurrent gene fusions as diagnostic markers and clinical targets for lung cancer.

### CROSS REFERENCE TO RELATED APPLICATIONS

This application claims priority to application Ser. No. 61/249,089, filed Oct. 6, 2009, which is herein incorporated by reference in its entirety.

### GOVERNMENT SUPPORT

This invention was made with government support under DA021519 awarded by the National Institutes of Health. The government has certain rights in the invention.

### FIELD OF THE INVENTION

The present invention relates to compositions and methods for cancer diagnosis, research and therapy, including but not limited to, cancer markers. In particular, the present invention relates to recurrent gene fusions as diagnostic markers and clinical targets for lung cancer.

### BACKGROUND OF THE INVENTION

A central aim in cancer research is to identify altered genes that are causally implicated in oncogenesis. Several types of somatic mutations have been identified including base substitutions, insertions, deletions, translocations, and chromosomal gains and losses, all of which result in altered activity of an oncogene or tumor suppressor gene. First hypothesized in the early 1900's, there is now compelling evidence for a causal role for chromosomal rearrangements in cancer (Rowley, Nat Rev Cancer 1: 245 (2001)). Recurrent chromosomal aberrations were thought to be primarily characteristic of leukemias, lymphomas, and sarcomas. Epithelial tumors (carcinomas), which are much more common and contribute to a relatively large fraction of the morbidity and mortality associated with human cancer, comprise less than 1% of the known, disease-specific chromosomal rearrangements (Mitelman, Mutat Res 462: 247 (2000)). While hematological malignancies are often characterized by balanced, disease-specific chromosomal rearrangements, most solid tumors have a plethora of non-specific chromosomal aberrations. It is thought that the karyotypic complexity of solid tumors is due to secondary alterations acquired through cancer evolution or progression.

Two primary mechanisms of chromosomal rearrangements have been described. In one mechanism, promoter/enhancer elements of one gene are rearranged adjacent to a proto-oncogene, thus causing altered expression of an oncogenic protein. This type of translocation is exemplified by the apposition of immunoglobulin (IG) and T-cell receptor (TCR) genes to MYC leading to activation of this oncogene in B- and T-cell malignancies, respectively (Rabbitts, Nature 372: 143 (1994)). In the second mechanism, rearrangement results in the fusion of two genes, which produces a fusion protein that may have a new function or altered activity. The prototypic example of this translocation is the BCR-ABL gene fusion in chronic myelogenous leukemia (CML) (Rowley, Nature 243: 290 (1973); de Klein et al., Nature 300: 765 (1982)). Importantly, this finding led to the rational development of imatinib mesylate (Gleevec), which successfully targets the BCR-ABL kinase (Deininger et al., Blood 105: 2640 (2005)). Thus, identifying recurrent gene rearrangements in common epithelial tumors may have profound implications for cancer drug discovery efforts as well as patient treatment.

### SUMMARY OF THE INVENTION

The present invention relates to compositions and methods for cancer diagnosis, research and therapy, including but not limited to, cancer markers. In particular, the present invention relates to recurrent gene fusions as diagnostic markers and clinical targets for lung cancer.

For example, in some embodiments, the present invention provides a method for identifying lung cancer in a patient comprising: providing a sample from the patient; and detecting the presence or absence in the sample of a gene fusion having a 5′ portion from a transcriptional regulatory region of an R3HDM2 gene and a 3′ portion from a NFE2 gene, wherein detecting the presence in the sample of the gene fusion identifies lung cancer in the patient. In some embodiments, the transcriptional regulatory region of the R3HDM2 gene comprises a promoter region of the R3HDM2 gene. In some embodiments, the detecting step comprises detecting chromosomal rearrangements of genomic DNA having a 5′ DNA portion from the transcriptional regulatory region of the R3HDM2 gene and a 3′ DNA portion from the NFE2 gene. In some embodiments, the detecting step comprises detecting chimeric mRNA transcripts having a 5′ RNA portion transcribed from the transcriptional regulatory region of the R3HDM2 gene and a 3′ RNA portion transcribed from a NFE2 gene. In some embodiments, the sample is tissue, blood, plasma, serum or lung cells.

In additional embodiments, the present invention provides a composition comprising at least one of the following: (a) an oligonucleotide probe comprising a sequence that hybridizes to a junction of a chimeric genomic DNA or chimeric mRNA in which a 5′ portion of the chimeric genomic DNA or chimeric mRNA is from a transcriptional regulatory region of an R3HDM2 gene and a 3′ portion of the chimeric genomic DNA or chimeric mRNA is from a NFE2 gene; (b) a first oligonucleotide probe comprising a sequence that hybridizes to a 5′ portion of a chimeric genomic DNA or chimeric mRNA from a transcriptional regulatory region of an R3HDM2 gene and a second oligonucleotide probe comprising a sequence that hybridizes to a 3′ portion of the chimeric genomic DNA or chimeric mRNA from a NFE2 gene; (c) a first amplification oligonucleotide comprising a sequence that hybridizes to a 5′ portion of a chimeric genomic DNA or chimeric mRNA from a transcriptional regulatory region of an R3HDM2 gene and a second amplification oligonucleotide comprising a sequence that hybridizes to a 3′ portion of the chimeric genomic DNA or chimeric mRNA from a NFE2 gene; (d) an antibody to a chimeric protein having an animo-terminal portion encoded by the R3HDM2 gene and a carboxy-terminal portion encoded by a NFE2 gene; or (e) an antibody to an overexpressed NFE2 gene.

### DESCRIPTION OF THE FIGURES

FIG. 1. Exploring cancer-related gene fusions in the context of known molecular interaction networks. (a) The hypergeometric statistics for the interrogation of the fusion gene groups defined by shared partners with molecular interaction gene sets, defining the significance of overlap between a set of fusion genes i (e.g., all BCR partners) and a set of interacting genes j (e.g., all PIK3R1 interacting genes). (b) The total number of significant links (589) and the number of fusion partner groups having these links (33) were plotted with the distribution calculated from randomly chosen gene sets with equal amount of connectivity (1000 permutations). (c) Analysis of the fusion partner groups with a compendium of molecular concepts by hypergeometric statistics. (d) By setting a p value threshold of 10⁻⁷, the fusion-interaction network was resolved and assembled into six major clusters. (i) Acute lymphoblastic lymphoma (ALL) clusters with a hub of GATA3; (ii) Acute/chronic myelogenous leukemia (AML and CML) clusters with a hub of MEIS1; (iii) B cell lymphoma and chronic lymphoblastic lymphoma (CLL) cluster through the hubs of CDK6, and CTNNB1; (iv) AML fusions partially focusing on HDAC1; RUNX1 is the hub of immunoglobulin fusions, and also involved in multiple fusions in AML, thus links cluster iii and iv. (v) ALL and CML cluster through the hub of PIK3R1; (vi) Sarcoma and prostate cancer cluster around ERG and MSK1. The PI3K3R1 and HDAC1 hubs are highlighted in dashed line regions.

FIG. 2. Distinguishing biological features of gene fusions and point mutations in cancer. (a) Enrichment analysis with a compendium of molecular concepts generates two sets of minimally-overlapping signature concepts for fusion and point mutation genes. (b) The ConSig algorithm. (c) Plotting the fusion and mutation ConSig-score against each other produced a segregation of known fusion and mutation genes. (d) Identifying the top 60 genes rated by rConSig-score produced a list highly enriched for established cancer genes.

FIG. 3. Characterizing the genomic imbalances of recurrent gene fusions in acute lymphocytic leukemia. A SNP array dataset was used to evaluate genomic aberrations causing gene fusions in acute lymphocytic leukemia (ALL). (a) The recurrent TCF3-PBX1 fusion (n=17) was associated with deletion of the 3′ region of TCF3 and duplication of the 3′ region of PBX1. (b) Of the 56 samples with unbalanced gene fusions in this dataset, 55 samples conformed to the fusion breakpoint principle. (c) Unbalanced fusions in the 12 leukemia cancer cell lines follow the fusion breakpoint principle.

FIG. 4. Discovery and validation of the R3HDM2-NFE2 fusion using the ConSig algorithm. (a) Pair-end transcriptome sequencing of the H2228 lung cancer cell line. Left, rating the 3′ partners of paired-end chimeras (≧3 paired reads) by rConSig score prioritizes EML4-ALK as a candidate fusion in the H2228 lung cancer cell line (known to harbor this fusion), which was supported by six paired reads. Right, ConSig analysis nominates the R3HDM2-NFE2 fusion as the top candidate in the H1792 lung cancer cell line. (b) Schematic of the R3HDM2-NFE2 fusion mRNA and protein. (c) The R3HDM2-NFE2 fusion was confirmed by RT-PCR and sequencing of the PCR product. (d) Analysis of SNP array data from 139 lung adenocarcinoma tissues revealed recurrent copy number aberrations in 2 patients at the 3′ NFE2 locus, as well as the focal amplification of R3HDM2-NFE2 fusion on H1792. (e) Left, schematic of the genomic organization of R3HDM2-NFE2 fusion. (f) As in (e), except the data from three lung adenocarcinoma patients.

FIG. 5. The domain architectures of known gene fusions. Domains for known fusion genes were clustered according to their sequence similarity (columns), while the gene fusions were clustered according to their domain similarity (rows).

FIG. 6. Kolmogorov-Smirnov (K-S) analysis for the known cancer genes based on the rConSig-score with or without the pathways significantly overlapping with the molecular interactions (p<0.01).

FIG. 7. The D-line y=kx (k=1.67) of the fusion-mutation ConSig plot was determined by setting optimal separation capacity. The D-line separates 85% of mutation genes from 80% of fusion genes.

FIG. 8. The signature molecular concepts for 5′ and 3′ fusion genes. The enrichment analysis of 5′ or 3′ fusion genes against all molecular concepts was done by Fisher's exact tests. As a result, 3′ fusion genes demonstrated much more enriched “signature concepts”. The p value cutoff was p<10⁻⁵ for 5′ fusion genes, and p<10⁻⁶ for 3′ fusion genes.

FIG. 9. The fusion breakpoint principle and the confirming evidence. (a) The fusion breakpoint principle. The left panel illustrates the application of the principle to inter-chromosomal translocations generating unbalanced gene fusions; the middle panel illustrates the inferred pattern of genomic imbalances for intra-chromosome gene fusions resulting from a single chromosome rearrangement with three different gene placements; the right panel shows the complex pattern of genomic imbalances for intra-chromosome translocations resulting from multiple rearrangements. (b) Evidence for the principle from analysis of independent datasets and literature curation. (c) Representative FISH results of the unbalanced ETS transcription factor fusions in 238 prostate cancer patients (University of Michigan Cohort).

FIG. 10. Complex chromosome rearrangements generate contradictory cases to the breakpoint principle on array CGH, but not on FISH data. Upper panel shows the location of FISH probes on the genomic loci of 5′ or 3′ partner genes. The middle panel shows the FISH appearance of complex chromosome rearrangements resulting in a balanced fusion (split signal) and an unbalanced translocation (from left to right: 3′ duplication, 5′ deletion, 5′ duplication, 3′ deletion). The lower panel shows the relative quantification of DNA copy number data generated by microarray CGH analysis from the genomic regions 1 Mb apart from the fusion genes. The x axis indicates the physical position of the genomic aberrations. The fusion partners are indicated by arrows.

FIG. 11. Gene expression profile of R3HDM2 and NFE2. (a) Microarray expression data of R3HDM2 and NFE2 on lung cancer cell lines. (b) The expression of NFE2 in 40 distinct normal tissues using Oncomine

FIG. 12. Exon-walking RT-PCR reveals specific overexpression of NFE2 coding exons. Exon-walking qRT-PCR with primer pairs corresponding to the indicated exons were used to evaluate exon-level expression changes in NFE2.

FIG. 13. FISH analysis of NFE2 and R3HDM2 loci by split probes strategy on selected lung cancer cell lines. Upper, the genomic organizations of NFE2 and R3HDM2 loci. Lower, interphase FISH analysis with NFE2 and R3HDM2 split probes showing normal co-localizing signals on H1975, H838, H358, and H1993 cell lines.

FIG. 14. WST-1 assay shows inhibited cell proliferation after the NFE2 knockdown on H1792 cell line, but not on the control cell line H460. (a). NFE2 knockdown inhibits cell proliferation on H1792 cell line expressing the R3HDM3-NFE2 fusion by a WST-1 assay (absorbance at 490 nm was measured). (b) NFE2 knockdown on H460 cell line with low level NFE2 expression did not have significant effect on cell proliferation.

### DEFINITIONS

To facilitate an understanding of the present invention, a number of terms and phrases are defined below:

As used herein, the term “gene fusion” refers to a chimeric genomic DNA, a chimeric messenger RNA, a truncated protein or a chimeric protein resulting from the fusion of at least a portion of a first gene to at least a portion of a second gene. The gene fusion need not include entire genes or exons of genes.

As used herein, the term “gene upregulated in cancer” refers to a gene that is expressed (e.g., mRNA or protein expression) at a higher level in cancer (e.g., lung cancer) relative to the level in other tissues. In some embodiments, genes upregulated in cancer are expressed at a level at least 10%, preferably at least 25%, even more preferably at least 50%, still more preferably at least 100%, yet more preferably at least 200%, and most preferably at least 300% higher than the level of expression in other tissues.

As used herein, the term “gene upregulated in lung tissue” refers to a gene that is expressed (e.g., mRNA or protein expression) at a higher level in lung tissue relative to the level in other tissue. In some embodiments, genes upregulated in lung tissue are expressed at a level at least 10%, preferably at least 25%, even more preferably at least 50%, still more preferably at least 100%, yet more preferably at least 200%, and most preferably at least 300% higher than the level of expression in other tissues. In some embodiments, genes upregulated in lung tissue are exclusively expressed in lung tissue.

As used herein, the term “transcriptional regulatory region” refers to the region of a gene comprising sequences that modulate (e.g., upregulate or downregulate) expression of the gene. In some embodiments, the transcriptional regulatory region of a gene comprises non-coding upstream sequence of a gene, also called the 5′ untranslated region (5′UTR). In other embodiments, the transcriptional regulatory region contains sequences located within the coding region of a gene or within an intron (e.g., enhancers).

As used herein, the terms “detect”, “detecting” or “detection” may describe either the general act of discovering or discerning or the specific observation of a detectably labeled composition.

As used herein, the term “stage of cancer” refers to a qualitative or quantitative assessment of the level of advancement of a cancer. Criteria used to determine the stage of a cancer include, but are not limited to, the size of the tumor and the extent of metastases (e.g., localized or distant).

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to at least a portion of another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

### DETAILED DESCRIPTION OF THE INVENTION

The present invention is based on the discovery of recurrent gene fusions in lung cancer. The present invention provides diagnostic, research, and therapeutic methods that either directly or indirectly detect or target the gene fusions. The present invention also provides compositions for diagnostic, research, and therapeutic purposes.

By undertaking a comprehensive analysis of the biological associations of all genes contributing to gene fusions, it is demonstrated that, while analysis of domain architectures and shared pathways was less informative, cancer-related fusion genes tend to engage distinct interaction networks or share common gene ontologies. Using such information, this finding was applied to a genomic scale and an algorithm called “concept signature” score or “ConSig” score was developed to assay the probability that any given gene contributes to a driving gene fusion based on the strength of that gene's association with biological concepts characteristic of cancer genes. To integrate use of high-throughput genomic data, the chromosomal imbalances associated with gene fusions were characterized and it was found that recurrent gene fusions exhibit distinctive patterns of copy number alteration corresponding to differential portions of fusion partners.

The ConSig score was applied to NGS transcriptome data to benchmark fusion candidates, which were then assessed for chromosomal aberrations complying with the fusion breakpoint principle by integrating high-quality copy number data. The ConSig score was able to identify the known EML4-ALK fusion as the top-ranked candidate in the H2228 lung cancer cell line, and, in addition, further evidence of a R3HDM2-NFE2 fusion in H1792 cell line was found. It was shown that the R3HDM2-NFE2 fusion, which results in overexpression of wild-type NFE2, promotes cell proliferation and invasion. Moreover, through analysis of SNP arrays and lung TMAs, it was found that chromosomal rearrangements at the NFE2 locus are recurrent in a small subset of patient tumors, indicating that NFE2 may contribute to a new class of lung cancer molecular biology. These methods are further supported by the observation of an oncogenic UBE2L3-KRAS fusion transcript in 30-40% of prostate tumors, which was nominated by similar analytical approaches. These data indicate that such approaches have broad applicability to the analysis of multi-dimensional cancer genomic data.

### I. Gene Fusions

The present invention identifies recurrent gene fusions indicative of lung cancer. In some embodiments, the gene fusions are the result of a chromosomal rearrangement of an transcriptional regulatory region of a first gene (e.g., R3HDM2) and NFE2. The gene fusions typically comprise a 5′ portion from a transcriptional regulatory region of first gene (e.g., R3HDM2) and a 3′ portion from NFE2. In some embodiments, expression of RSHDM2-NFE2 fusions results in overexpression of wild type NFE2 protein. The recurrent gene fusions have use as diagnostic markers and clinical targets for lung cancer.

Homo sapiens R3H domain containing 2 (R3HDM2) has an mRNA sequence described by Genbank accession No. NM_{—}014925. The most prominent feature of the R3H motif is the presence of an invariant arginine residue and a highly conserved histidine residue that are separated by three residues. The motif also displays a conserved pattern of hydrophobic residues, prolines and glycines.

The sequences that contain the R3H domain, many of which are hypothetical proteins predicted from genome sequencing projects, can be grouped into eight families on the basis of similarities outside the R3H region. Three of the families contain ATPase domains either upstream (families II and VII) or downstream of the R3H domain (family VIII). The N-terminal part of members of family VII contains an SF1 helicase domain5. The C-terminal part of family VIII contains an SF2 DEAH helicase domain5. The ATPase domain in the members of family II is similar to the stage-III sporulation protein AA (S3AA_BACSU), the proteasome ATPase, bacterial transcription-termination factor r and the mitochondrial F1-ATPase b subunit (the F5 helicase family5). Family VI contains Cys-rich repeats6, as well as a ring-type zinc finger upstream of the R3H domain. JAG bacterial proteins (family I) contain a KH domain N-terminal to the R3H domain. The functions of other domains in R3H proteins support the hypothesis that the R3H domain is involved in interactions with single-stranded nucleic acids.

Nuclear Factor Erythroid 2 (NFE2) has an mRNA sequence described by Genbank accession no. NM_{—}001136023.

The 45-kD subunit of the human globin locus control region binding protein, NFE2, was cloned by homology to the murine gene. Immunoprecipitation experiments demonstrated in vivo association of the p45 subunit with an 18-kD protein. Because bZIP proteins bind DNA as dimers, it is likely that native NFE2 is a heterodimer of 45- and 18-kD subunits. Extensive survey of human tissue samples found that NFE2 expression is not limited to erythropoietic organs. Expression in the colon and testis indicated that NFE2 may participate in the regulation of genes other than globin.

By fluorescence in situ hybridization, Chan et al. (1995) confirmed the localization to 12q13.1-q13.3 and demonstrated that 2 genes of the same family of transcription factors with many similarities of gene structure, NFE2L1 and NFE2L2, are each located on other chromosomes. The 3 genes probably were derived from a single ancestor by chromosomal duplication inasmuch as other genes that also map to the 3 chromosomal regions are related to one another.

Peters et al. (Nature 362: 768-770, 1993) demonstrated that the Nfe2 gene in the mouse maps to chromosome 15 in a region containing the microcytic anemia (mk) gene. Homozygous mk mice were shown by Bannerman et al. (Brit. J. Haemat. 23: 235-245, 1972, 1972) to have defective intestinal iron transport and severe anemia. Peters et al. (supra, 1993) demonstrated Nfe2 expression in the mouse small intestine and NF-E2 binding activity in nuclear extracts of a human colon carcinoma cell line (Caco-2). Caco-2 cells possess properties of the small intestine, including the ability to transport iron. These data together indicated that NF-E2 plays a role in all aspects of hemoglobin production: globin synthesis, heme synthesis, and the procurement of iron. (NF-E2 recognition sites are present not only in the locus control regions of the globin genes but also in the gene promoters of 2 heme biosynthetic enzymes, porphobilinogen deaminase and ferrochelatase. As an essential factor for megakaryocyte maturation and platelet production, NF-E2 regulates critical target genes independent of the action of thrombopoietin.

### II. Antibodies

The gene fusion proteins of the present invention, including fragments, derivatives and analogs thereof, may be used as immunogens to produce antibodies having use in the diagnostic, research, and therapeutic methods described below. The antibodies may be polyclonal or monoclonal, chimeric, humanized, single chain or Fab fragments. Various procedures known to those of ordinary skill in the art may be used for the production and labeling of such antibodies and fragments. See, e.g., Burns, ed., Immunochemical Protocols, 3^{rd} ed., Humana Press (2005); Harlow and Lane, Antibodies: A Laboratory Manual, Cold Spring Harbor Laboratory (1988); Kozbor et al., Immunology Today 4: 72 (1983); Köhler and Milstein, Nature 256: 495 (1975). Antibodies or fragments exploiting the differences between the truncated ETS family member protein or chimeric protein and their respective native proteins are particularly preferred.

### III. Diagnostic Applications

The gene fusions described herein are detectable as DNA, RNA or protein. Initially, the gene fusion is detectable as a chromosomal rearrangement of genomic DNA having a 5′ portion from a first gene (e.g., R3HDM2) and a 3′ portion from a second gene (e.g., NFE2). Once transcribed, the gene fusion is detectable as a chimeric mRNA having a 5′ portion from R3HDM2 and a 3′ portion from NFE2. Once translated, the gene fusion is detectable as fusion of a 5′ portion from R3HDM2 and a 3′ portion from NFE2 or wild type NFE2. The proteins may differ from their respective native proteins in amino acid sequence, post-translational processing and/or secondary, tertiary or quaternary structure. Such differences, if present, can be used to identify the presence of the gene fusion. Specific methods of detection are described in more detail below.

The present invention provides DNA, RNA and protein based diagnostic methods that either directly or indirectly detect the gene fusions. The present invention also provides compositions and kits for diagnostic purposes.

The diagnostic methods of the present invention may be qualitative or quantitative. Quantitative diagnostic methods may be used, for example, to discriminate between indolent and aggressive cancers via a cutoff or threshold level. Where applicable, qualitative or quantitative diagnostic methods may also include amplification of target, signal or intermediary (e.g., a universal primer).

An initial assay may confirm the presence of a gene fusion but not identify the specific fusion. A secondary assay is then performed to determine the identity of the particular fusion, if desired. The second assay may use a different detection technology than the initial assay.

The gene fusions of the present invention may be detected along with other markers in a multiplex or panel format. Markers are selected for their predictive value alone or in combination with the gene fusions. Markers for other cancers, diseases, infections, and metabolic conditions are also contemplated for inclusion in a multiplex or panel format.

The diagnostic methods of the present invention may also be modified with reference to data correlating particular gene fusions with the stage, aggressiveness or progression of the disease or the presence or risk of metastasis. Ultimately, the information provided by the methods of the present invention will assist a physician in choosing the best course of treatment for a particular patient.

A. Sample

Any patient sample suspected of containing the gene fusions may be tested according to the methods of the present invention. By way of non-limiting examples, the sample may be tissue (e.g., a lung biopsy, blood, or other bodily fluid.

The patient sample typically requires preliminary processing designed to isolate or enrich the sample for the gene fusions or cells that contain the gene fusions. A variety of techniques known to those of ordinary skill in the art may be used for this purpose, including but not limited to: centrifugation; immunocapture; cell lysis; and, nucleic acid target capture (See, e.g., EP Pat. No. 1 409 727, herein incorporated by reference in its entirety).

B. DNA and RNA Detection

The gene fusions of the present invention may be detected as chromosomal rearrangements of genomic DNA or chimeric mRNA using a variety of nucleic acid techniques known to those of ordinary skill in the art, including but not limited to: nucleic acid sequencing; nucleic acid hybridization; and, nucleic acid amplification.

1. Sequencing

Illustrative non-limiting examples of nucleic acid sequencing techniques include, but are not limited to, chain terminator (Sanger) sequencing and dye terminator sequencing. Those of ordinary skill in the art will recognize that because RNA is less stable in the cell and more prone to nuclease attack experimentally RNA is usually reverse transcribed to DNA before sequencing.

Chain terminator sequencing uses sequence-specific termination of a DNA synthesis reaction using modified nucleotide substrates. Extension is initiated at a specific site on the template DNA by using a short radioactive, or other labeled, oligonucleotide primer complementary to the template at that region. The oligonucleotide primer is extended using a DNA polymerase, standard four deoxynucleotide bases, and a low concentration of one chain terminating nucleotide, most commonly a di-deoxynucleotide. This reaction is repeated in four separate tubes with each of the bases taking turns as the di-deoxynucleotide. Limited incorporation of the chain terminating nucleotide by the DNA polymerase results in a series of related DNA fragments that are terminated only at positions where that particular di-deoxynucleotide is used. For each reaction tube, the fragments are size-separated by electrophoresis in a slab polyacrylamide gel or a capillary tube filled with a viscous polymer. The sequence is determined by reading which lane produces a visualized mark from the labeled primer as you scan from the top of the gel to the bottom.

Dye terminator sequencing alternatively labels the terminators. Complete sequencing can be performed in a single reaction by labeling each of the di-deoxynucleotide chain-terminators with a separate fluorescent dye, which fluoresces at a different wavelength.

2. Hybridization

Illustrative non-limiting examples of nucleic acid hybridization techniques include, but are not limited to, in situ hybridization (ISH), microarray, and Southern or Northern blot.

In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. RNA ISH is used to measure and localize mRNAs and other transcripts within tissue sections or whole mounts. Sample cells and tissues are usually treated to fix the target transcripts in place and to increase access of the probe. The probe hybridizes to the target sequence at elevated temperature, and then the excess probe is washed away. The probe that was labeled with either radio-, fluorescent- or antigen-labeled bases is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

a. FISH

In some embodiments, fusion sequences are detected using fluorescence in situ hybridization (FISH). The preferred FISH assays for the present invention utilize bacterial artificial chromosomes (BACs). These have been used extensively in the human genome sequencing project (see Nature 409: 953-958 (2001)) and clones containing specific BACs are available through distributors that can be located through many sources, e.g., NCBI. Each BAC clone from the human genome has been given a reference name that unambiguously identifies it. These names can be used to find a corresponding GenBank sequence and to order copies of the clone from a distributor.

b. Microarrays

Different kinds of biological assays are called microarrays including, but not limited to: DNA microarrays (e.g., cDNA microarrays and oligonucleotide microarrays); protein microarrays; tissue microarrays; transfection or cell microarrays; chemical compound microarrays; and, antibody microarrays. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes by comparing gene expression in disease and normal cells. Microarrays can be fabricated using a variety of technologies, including but not limiting: printing with fine-pointed pins onto glass slides; photolithography using pre-made masks; photolithography using dynamic micromirror devices; ink-jet printing; or, electrochemistry on microelectrode arrays.

Southern and Northern blotting is used to detect specific DNA or RNA sequences, respectively. DNA or RNA extracted from a sample is fragmented, electrophoretically separated on a matrix gel, and transferred to a membrane filter. The filter bound DNA or RNA is subject to hybridization with a labeled probe complementary to the sequence of interest. Hybridized probe bound to the filter is detected. A variant of the procedure is the reverse Northern blot, in which the substrate nucleic acid that is affixed to the membrane is a collection of isolated DNA fragments and the probe is RNA extracted from a tissue and labeled.

3. Amplification

Chromosomal rearrangements of genomic DNA and chimeric mRNA may be amplified prior to or simultaneous with detection. Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

The polymerase chain reaction (U.S. Pat. Nos. 4,683,195, 4,683,202, 4,800,159 and 4,965,188, each of which is herein incorporated by reference in its entirety), commonly referred to as PCR, uses multiple cycles of denaturation, annealing of primer pairs to opposite strands, and primer extension to exponentially increase copy numbers of a target nucleic acid sequence. In a variation called RT-PCR, reverse transcriptase (RT) is used to make a complementary DNA (cDNA) from mRNA, and the cDNA is then amplified by PCR to produce multiple copies of DNA. For other various permutations of PCR see, e.g., U.S. Pat. Nos. 4,683,195, 4,683,202 and 4,800,159; Mullis et al., Meth. Enzymol. 155: 335 (1987); and, Murakawa et al., DNA 7: 287 (1988), each of which is herein incorporated by reference in its entirety.

Transcription mediated amplification (U.S. Pat. Nos. 5,480,784 and 5,399,491, each of which is herein incorporated by reference in its entirety), commonly referred to as TMA, synthesizes multiple copies of a target nucleic acid sequence autocatalytically under conditions of substantially constant temperature, ionic strength, and pH in which multiple RNA copies of the target sequence autocatalytically generate additional copies. See, e.g., U.S. Pat. Nos. 5,399,491 and 5,824,518, each of which is herein incorporated by reference in its entirety. In a variation described in U.S. Publ. No. 20060046265 (herein incorporated by reference in its entirety), TMA optionally incorporates the use of blocking moieties, terminating moieties, and other modifying moieties to improve TMA process sensitivity and accuracy.

The ligase chain reaction (Weiss, R., Science 254: 1292 (1991), herein incorporated by reference in its entirety), commonly referred to as LCR, uses two sets of complementary DNA oligonucleotides that hybridize to adjacent regions of the target nucleic acid. The DNA oligonucleotides are covalently linked by a DNA ligase in repeated cycles of thermal denaturation, hybridization and ligation to produce a detectable double-stranded ligated oligonucleotide product.

Strand displacement amplification (Walker, G. et al., Proc. Natl. Acad. Sci. USA 89: 392-396 (1992); U.S. Pat. Nos. 5,270,184 and 5,455,166, each of which is herein incorporated by reference in its entirety), commonly referred to as SDA, uses cycles of annealing pairs of primer sequences to opposite strands of a target sequence, primer extension in the presence of a dNTPaS to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product. Thermophilic SDA (tSDA) uses thermophilic endonucleases and polymerases at higher temperatures in essentially the same method (EP Pat. No. 0 684 315).

Other amplification methods include, for example: nucleic acid sequence based amplification (U.S. Pat. No. 5,130,238, herein incorporated by reference in its entirety), commonly referred to as NASBA; one that uses an RNA replicase to amplify the probe molecule itself (Lizardi et al., BioTechnol. 6: 1197 (1988), herein incorporated by reference in its entirety), commonly referred to as Qβ replicase; a transcription based amplification method (Kwoh et al., Proc. Natl. Acad. Sci. USA 86:1173 (1989)); and, self-sustained sequence replication (Guatelli et al., Proc. Natl. Acad. Sci. USA 87: 1874 (1990), each of which is herein incorporated by reference in its entirety). For further discussion of known amplification methods see Persing, David H., “In Vitro Nucleic Acid Amplification Techniques” in Diagnostic Medical Microbiology: Principles and Applications (Persing et al., Eds.), pp. 51-87 (American Society for Microbiology, Washington, DC (1993)).

4. Detection Methods

Non-amplified or amplified gene fusion nucleic acids can be detected by any conventional means. For example, the gene fusions can be detected by hybridization with a detectably labeled probe and measurement of the resulting hybrids. Illustrative non-limiting examples of detection methods are described below.

One illustrative detection method, the Hybridization Protection Assay (HPA) involves hybridizing a chemiluminescent oligonucleotide probe (e.g., an acridinium ester-labeled (AE) probe) to the target sequence, selectively hydrolyzing the chemiluminescent label present on unhybridized probe, and measuring the chemiluminescence produced from the remaining probe in a luminometer. See, e.g., U.S. Pat. No. 5,283,174 and Norman C. Nelson et al., Nonisotopic Probing, Blotting, and Sequencing, ch. 17 (Larry J. Kricka ed., 2d ed. 1995, each of which is herein incorporated by reference in its entirety).

Another illustrative detection method provides for quantitative evaluation of the amplification process in real-time. Evaluation of an amplification process in “real-time” involves determining the amount of amplicon in the reaction mixture either continuously or periodically during the amplification reaction, and using the determined values to calculate the amount of target sequence initially present in the sample. A variety of methods for determining the amount of initial target sequence present in a sample based on real-time amplification are well known in the art. These include methods disclosed in U.S. Pat. Nos. 6,303,305 and 6,541,205, each of which is herein incorporated by reference in its entirety. Another method for determining the quantity of target sequence initially present in a sample, but which is not based on a real-time amplification, is disclosed in U.S. Pat. No. 5,710,029, herein incorporated by reference in its entirety.

Amplification products may be detected in real-time through the use of various self-hybridizing probes, most of which have a stem-loop structure. Such self-hybridizing probes are labeled so that they emit differently detectable signals, depending on whether the probes are in a self-hybridized state or an altered state through hybridization to a target sequence. By way of non-limiting example, “molecular torches” are a type of self-hybridizing probe that includes distinct regions of self-complementarity (referred to as “the target binding domain” and “the target closing domain”) which are connected by a joining region (e.g., non-nucleotide linker) and which hybridize to each other under predetermined hybridization assay conditions. In a preferred embodiment, molecular torches contain single-stranded base regions in the target binding domain that are from 1 to about 20 bases in length and are accessible for hybridization to a target sequence present in an amplification reaction under strand displacement conditions. Under strand displacement conditions, hybridization of the two complementary regions, which may be fully or partially complementary, of the molecular torch is favored, except in the presence of the target sequence, which will bind to the single-stranded region present in the target binding domain and displace all or a portion of the target closing domain. The target binding domain and the target closing domain of a molecular torch include a detectable label or a pair of interacting labels (e.g., luminescent/quencher) positioned so that a different signal is produced when the molecular torch is self-hybridized than when the molecular torch is hybridized to the target sequence, thereby permitting detection of probe:target duplexes in a test sample in the presence of unhybridized molecular torches. Molecular torches and a variety of types of interacting label pairs are disclosed in U.S. Pat. No. 6,534,274, herein incorporated by reference in its entirety.

Another example of a detection probe having self-complementarity is a “molecular beacon.” Molecular beacons include nucleic acid molecules having a target complementary sequence, an affinity pair (or nucleic acid arms) holding the probe in a closed conformation in the absence of a target sequence present in an amplification reaction, and a label pair that interacts when the probe is in a closed conformation. Hybridization of the target sequence and the target complementary sequence separates the members of the affinity pair, thereby shifting the probe to an open conformation. The shift to the open conformation is detectable due to reduced interaction of the label pair, which may be, for example, a fluorophore and a quencher (e.g., DABCYL and EDANS). Molecular beacons are disclosed in U.S. Pat. Nos. 5,925,517 and 6,150,097, herein incorporated by reference in its entirety.

Other self-hybridizing probes are well known to those of ordinary skill in the art. By way of non-limiting example, probe binding pairs having interacting labels, such as those disclosed in U.S. Pat. No. 5,928,862 (herein incorporated by reference in its entirety) might be adapted for use in the present invention. Probe systems used to detect single nucleotide polymorphisms (SNPs) might also be utilized in the present invention. Additional detection systems include “molecular switches,” as disclosed in U.S. Publ. No. 20050042638, herein incorporated by reference in its entirety. Other probes, such as those comprising intercalating dyes and/or fluorochromes, are also useful for detection of amplification products in the present invention. See, e.g., U.S. Pat. No. 5,814,447 (herein incorporated by reference in its entirety).

C. Protein Detection

The gene fusions of the present invention may be detected as truncated or chimeric proteins using a variety of protein techniques known to those of ordinary skill in the art, including but not limited to: protein sequencing; and, immunoassays.

1. Sequencing

Illustrative non-limiting examples of protein sequencing techniques include, but are not limited to, mass spectrometry and Edman degradation.

Mass spectrometry can, in principle, sequence any size protein but becomes computationally more difficult as size increases. A protein is digested by an endoprotease, and the resulting solution is passed through a high pressure liquid chromatography column. At the end of this column, the solution is sprayed out of a narrow nozzle charged to a high positive potential into the mass spectrometer. The charge on the droplets causes them to fragment until only single ions remain. The peptides are then fragmented and the mass-charge ratios of the fragments measured. The mass spectrum is analyzed by computer and often compared against a database of previously sequenced proteins in order to determine the sequences of the fragments. The process is then repeated with a different digestion enzyme, and the overlaps in sequences are used to construct a sequence for the protein.

In the Edman degradation reaction, the peptide to be sequenced is adsorbed onto a solid surface (e.g., a glass fiber coated with polybrene). The Edman reagent, phenylisothiocyanate (PTC), is added to the adsorbed peptide, together with a mildly basic buffer solution of 12% trimethylamine, and reacts with the amine group of the N-terminal amino acid. The terminal amino acid derivative can then be selectively detached by the addition of anhydrous acid. The derivative isomerizes to give a substituted phenylthiohydantoin, which can be washed off and identified by chromatography, and the cycle can be repeated. The efficiency of each step is about 98%, which allows about 50 amino acids to be reliably determined.

2. Immunoassays

Illustrative non-limiting examples of immunoassays include, but are not limited to: immunoprecipitation; Western blot; ELISA; immunohistochemistry; immunocytochemistry; flow cytometry; and, immuno-PCR. Polyclonal or monoclonal antibodies detectably labeled using various techniques known to those of ordinary skill in the art (e.g., colorimetric, fluorescent, chemiluminescent or radioactive) are suitable for use in the immunoassays.

Immunoprecipitation is the technique of precipitating an antigen out of solution using an antibody specific to that antigen. The process can be used to identify protein complexes present in cell extracts by targeting a protein believed to be in the complex. The complexes are brought out of solution by insoluble antibody-binding proteins isolated initially from bacteria, such as Protein A and Protein G. The antibodies can also be coupled to sepharose beads that can easily be isolated out of solution. After washing, the precipitate can be analyzed using mass spectrometry, Western blotting, or any number of other methods for identifying constituents in the complex.

A Western blot, or immunoblot, is a method to detect protein in a given sample of tissue homogenate or extract. It uses gel electrophoresis to separate denatured proteins by mass. The proteins are then transferred out of the gel and onto a membrane, typically polyvinyldiflroride or nitrocellulose, where they are probed using antibodies specific to the protein of interest. As a result, researchers can examine the amount of protein in a given sample and compare levels between several groups.

An ELISA, short for Enzyme-Linked ImmunoSorbent Assay, is a biochemical technique to detect the presence of an antibody or an antigen in a sample. It utilizes a minimum of two antibodies, one of which is specific to the antigen and the other of which is coupled to an enzyme. The second antibody will cause a chromogenic or fluorogenic substrate to produce a signal. Variations of ELISA include sandwich ELISA, competitive ELISA, and ELISPOT. Because the ELISA can be performed to evaluate either the presence of antigen or the presence of antibody in a sample, it is a useful tool both for determining serum antibody concentrations and also for detecting the presence of antigen.

Immunohistochemistry and immunocytochemistry refer to the process of localizing proteins in a tissue section or cell, respectively, via the principle of antigens in tissue or cells binding to their respective antibodies. Visualization is enabled by tagging the antibody with color producing or fluorescent tags. Typical examples of color tags include, but are not limited to, horseradish peroxidase and alkaline phosphatase. Typical examples of fluorophore tags include, but are not limited to, fluorescein isothiocyanate (FITC) or phycoerythrin (PE).

Flow cytometry is a technique for counting, examining and sorting microscopic particles suspended in a stream of fluid. It allows simultaneous multiparametric analysis of the physical and/or chemical characteristics of single cells flowing through an optical/electronic detection apparatus. A beam of light (e.g., a laser) of a single frequency or color is directed onto a hydrodynamically focused stream of fluid. A number of detectors are aimed at the point where the stream passes through the light beam; one in line with the light beam (Forward Scatter or FSC) and several perpendicular to it (Side Scatter (SSC) and one or more fluorescent detectors). Each suspended particle passing through the beam scatters the light in some way, and fluorescent chemicals in the particle may be excited into emitting light at a lower frequency than the light source. The combination of scattered and fluorescent light is picked up by the detectors, and by analyzing fluctuations in brightness at each detector, one for each fluorescent emission peak, it is possible to deduce various facts about the physical and chemical structure of each individual particle. FSC correlates with the cell volume and SSC correlates with the density or inner complexity of the particle (e.g., shape of the nucleus, the amount and type of cytoplasmic granules or the membrane roughness).

Immuno-polymerase chain reaction (IPCR) utilizes nucleic acid amplification techniques to increase signal generation in antibody-based immunoassays. Because no protein equivalence of PCR exists, that is, proteins cannot be replicated in the same manner that nucleic acid is replicated during PCR, the only way to increase detection sensitivity is by signal amplification. The target proteins are bound to antibodies which are directly or indirectly conjugated to oligonucleotides. Unbound antibodies are washed away and the remaining bound antibodies have their oligonucleotides amplified. Protein detection occurs via detection of amplified oligonucleotides using standard nucleic acid detection methods, including real-time methods.

D. Data Analysis

In some embodiments, a computer-based analysis program is used to translate the raw data generated by the detection assay (e.g., the presence, absence, or amount of a given gene fusion or other markers) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., likelihood of cancer being present) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease.

E. In Vivo Imaging

The gene fusions of the present invention may also be detected using in vivo imaging techniques, including but not limited to: radionuclide imaging; positron emission tomography (PET); computerized axial tomography, X-ray or magnetic resonance imaging method, fluorescence detection, and chemiluminescent detection. In some embodiments, in vivo imaging techniques are used to visualize the presence of or expression of cancer markers in an animal (e.g., a human or non-human mammal). For example, in some embodiments, cancer marker mRNA or protein is labeled using a labeled antibody specific for the cancer marker. A specifically bound and labeled antibody can be detected in an individual using an in vivo imaging method, including, but not limited to, radionuclide imaging, positron emission tomography, computerized axial tomography, X-ray or magnetic resonance imaging method, fluorescence detection, and chemiluminescent detection. Methods for generating antibodies to the cancer markers of the present invention are described below.

The in vivo imaging methods of the present invention are useful in the diagnosis of cancers that express the cancer markers of the present invention (e.g., lung cancer). In vivo imaging is used to visualize the presence of a marker indicative of the cancer. Such techniques allow for diagnosis without the use of an unpleasant biopsy. The in vivo imaging methods of the present invention are also useful for providing prognoses to cancer patients. For example, the presence of a marker indicative of cancers likely to metastasize can be detected. The in vivo imaging methods of the present invention can further be used to detect metastatic cancers in other parts of the body.

In some embodiments, reagents (e.g., antibodies) specific for the cancer markers of the present invention are fluorescently labeled. The labeled antibodies are introduced into a subject (e.g., orally or parenterally). Fluorescently labeled antibodies are detected using any suitable method (e.g., using the apparatus described in U.S. Pat. No. 6,198,107, herein incorporated by reference).

In other embodiments, antibodies are radioactively labeled. The use of antibodies for in vivo diagnosis is well known in the art. Sumerdon et al., (Nucl. Med. Biol 17:247-254 [1990] have described an optimized antibody-chelator for the radioimmunoscintographic imaging of tumors using Indium-111 as the label. Griffin et al., (J Clin Onc 9:631-640 [1991]) have described the use of this agent in detecting tumors in patients suspected of having recurrent colorectal cancer. The use of similar agents with paramagnetic ions as labels for magnetic resonance imaging is known in the art (Lauffer, Magnetic Resonance in Medicine 22:339-342 [1991]). The label used will depend on the imaging modality chosen. Radioactive labels such as Indium-111, Technetium-99m, or Iodine-131 can be used for planar scans or single photon emission computed tomography (SPECT). Positron emitting labels such as Fluorine-19 can also be used for positron emission tomography (PET). For MRI, paramagnetic ions such as Gadolinium(III) or Manganese(II) can be used.

Radioactive metals with half-lives ranging from 1 hour to 3.5 days are available for conjugation to antibodies, such as scandium-47 (3.5 days) gallium-67 (2.8 days), gallium-68 (68 minutes), technetiium-99m (6 hours), and indium-111 (3.2 days), of which gallium-67, technetium-99m, and indium-111 are preferable for gamma camera imaging, gallium-68 is preferable for positron emission tomography.

A useful method of labeling antibodies with such radiometals is by means of a bifunctional chelating agent, such as diethylenetriaminepentaacetic acid (DTPA), as described, for example, by Khaw et al. (Science 209:295 [1980]) for In-111 and Tc-99m, and by Scheinberg et al. (Science 215:1511 [1982]). Other chelating agents may also be used, but the 1-(p-carboxymethoxybenzyl)EDTA and the carboxycarbonic anhydride of DTPA are advantageous because their use permits conjugation without affecting the antibody's immunoreactivity substantially.

Another method for coupling DPTA to proteins is by use of the cyclic anhydride of DTPA, as described by Hnatowich et al. (Int. J. Appl. Radiat. Isot. 33:327 [1982]) for labeling of albumin with In-111, but which can be adapted for labeling of antibodies. A suitable method of labeling antibodies with Tc-99m which does not use chelation with DPTA is the pretinning method of Crockford et al., (U.S. Pat. No. 4,323,546, herein incorporated by reference).

A preferred method of labeling immunoglobulins with Tc-99m is that described by Wong et al. (Int. J. Appl. Radiat. Isot., 29:251 [1978]) for plasma protein, and recently applied successfully by Wong et al. (J. Nucl. Med., 23:229 [1981]) for labeling antibodies.

In the case of the radiometals conjugated to the specific antibody, it is likewise desirable to introduce as high a proportion of the radiolabel as possible into the antibody molecule without destroying its immunospecificity. A further improvement may be achieved by effecting radiolabeling in the presence of the specific cancer marker of the present invention, to insure that the antigen binding site on the antibody will be protected. The antigen is separated after labeling.

In still further embodiments, in vivo biophotonic imaging (Xenogen, Almeda, Calif.) is utilized for in vivo imaging. This real-time in vivo imaging utilizes luciferase. The luciferase gene is incorporated into cells, microorganisms, and animals (e.g., as a fusion protein with a cancer marker of the present invention). When active, it leads to a reaction that emits light. A CCD camera and software is used to capture the image and analyze it.

F. Compositions & Kits

Any of these compositions, alone or in combination with other compositions of the present invention, may be provided in the form of a kit. For example, the single labeled probe and pair of amplification oligonucleotides may be provided in a kit for the amplification and detection of gene fusions of the present invention. Kits may further comprise appropriate controls and/or detection reagents. The probe and antibody compositions of the present invention may also be provided in the form of an array.

Compositions for use in the diagnostic methods of the present invention include, but are not limited to, probes, amplification oligonucleotides, and antibodies. Particularly preferred compositions detect a product only when a R3HDM2 gene fuses to a NFE2 gene. These compositions include: a single labeled probe comprising a sequence that hybridizes to the junction at which a 5′ portion from a R3HDM2 gene fuses to a 3′ portion from a NFE2 gene (i.e., spans the gene fusion junction); a pair of amplification oligonucleotides wherein the first amplification oligonucleotide comprises a sequence that hybridizes to a transcriptional regulatory region of a 5′ portion from a R3HDM2 gene fuses to a 3′ portion from a NFE2 gene; an antibody to an overexpressed NFE2 protein or portion thereof, an antibody to a chimeric protein having an amino-terminal portion from a R3HDM2 gene and a carboxy-terminal portion from a NFE2 gene. Other useful compositions, however, include: a pair of labeled probes wherein the first labeled probe comprises a sequence that hybridizes to a transcriptional regulatory region of a R3HDM2 gene and the second labeled probe comprises a sequence that hybridizes to a NFE2 gene.

### IV. Drug Screening Applications

In some embodiments, the present invention provides drug screening assays (e.g., to screen for anticancer drugs). The screening methods of the present invention utilize cancer markers identified using the methods of the present invention (e.g., including but not limited to, gene fusions of the present invention). For example, in some embodiments, the present invention provides methods of screening for compounds that alter (e.g., decrease) the expression of gene fusions. The compounds or agents may interfere with transcription, by interacting, for example, with the promoter region. The compounds or agents may interfere with mRNA produced from the fusion (e.g., by RNA interference, antisense technologies, etc.). The compounds or agents may interfere with pathways that are upstream or downstream of the biological activity of the fusion. In some embodiments, candidate compounds are antisense or interfering RNA agents (e.g., oligonucleotides) directed against cancer markers. In other embodiments, candidate compounds are antibodies or small molecules that specifically bind to a cancer marker regulator or expression products of the present invention and inhibit its biological function.

In one screening method, candidate compounds are evaluated for their ability to alter cancer marker expression by contacting a compound with a cell expressing a cancer marker and then assaying for the effect of the candidate compounds on expression. In some embodiments, the effect of candidate compounds on expression of a cancer marker gene is assayed for by detecting the level of cancer marker mRNA expressed by the cell. mRNA expression can be detected by any suitable method.

In other embodiments, the effect of candidate compounds on expression of cancer marker genes is assayed by measuring the level of polypeptide encoded by the cancer markers. The level of polypeptide expressed can be measured using any suitable method, including but not limited to, those disclosed herein.

Specifically, the present invention provides screening methods for identifying modulators, i.e., candidate or test compounds or agents (e.g., proteins, peptides, peptidomimetics, peptoids, small molecules or other drugs) which bind to cancer markers of the present invention, have an inhibitory (or stimulatory) effect on, for example, cancer marker expression or cancer marker activity, or have a stimulatory or inhibitory effect on, for example, the expression or activity of a cancer marker substrate. Compounds thus identified can be used to modulate the activity of target gene products (e.g., cancer marker genes) either directly or indirectly in a therapeutic protocol, to elaborate the biological function of the target gene product, or to identify compounds that disrupt normal target gene interactions. Compounds that inhibit the activity or expression of cancer markers are useful in the treatment of proliferative disorders, e.g., cancer, particularly lung cancer.

In one embodiment, the invention provides assays for screening candidate or test compounds that are substrates of a cancer marker protein or polypeptide or a biologically active portion thereof. In another embodiment, the invention provides assays for screening candidate or test compounds that bind to or modulate the activity of a cancer marker protein or polypeptide or a biologically active portion thereof.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; see, e.g., Zuckennann et al., J. Med. Chem. 37: 2678-85 [1994]); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 [1993]; Erb et al., Proc. Nad. Acad. Sci. USA 91:11422 [1994]; Zuckermann et al., J. Med. Chem. 37:2678 [1994]; Cho et al., Science 261:1303 [1993]; Carrell et al., Angew. Chem. Int. Ed. Engl. 33.2059 [1994]; Carell et al., Angew. Chem. Int. Ed. Engl. 33:2061 [1994]; and Gallop et al., J. Med. Chem. 37:1233 [1994].

Libraries of compounds may be presented in solution (e.g., Houghten, Biotechniques 13:412-421 [1992]), or on beads (Lam, Nature 354:82-84 [1991]), chips (Fodor, Nature 364:555-556 [1993]), bacteria or spores (U.S. Pat. No. 5,223,409; herein incorporated by reference), plasmids (Cull et al., Proc. Nad. Acad. Sci. USA 89:18651869 [1992]) or on phage (Scott and Smith, Science 249:386-390 [1990]; Devlin Science 249:404-406 [1990]; Cwirla et al., Proc. NatI. Acad. Sci. 87:6378-6382 [1990]; Felici, J. Mol. Biol. 222:301 [1991]).

In one embodiment, an assay is a cell-based assay in which a cell that expresses a cancer marker mRNA or protein or biologically active portion thereof is contacted with a test compound, and the ability of the test compound to the modulate cancer marker's activity is determined. Determining the ability of the test compound to modulate cancer marker activity can be accomplished by monitoring, for example, changes in enzymatic activity, destruction or mRNA, or the like.

The ability of the test compound to modulate cancer marker binding to a compound, e.g., a cancer marker substrate or modulator, can also be evaluated. This can be accomplished, for example, by coupling the compound, e.g., the substrate, with a radioisotope or enzymatic label such that binding of the compound, e.g., the substrate, to a cancer marker can be determined by detecting the labeled compound, e.g., substrate, in a complex.

Alternatively, the cancer marker is coupled with a radioisotope or enzymatic label to monitor the ability of a test compound to modulate cancer marker binding to a cancer marker substrate in a complex. For example, compounds (e.g., substrates) can be labeled with ¹²⁵I, ³⁵S ¹⁴C or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound (e.g., a cancer marker substrate) to interact with a cancer marker with or without the labeling of any of the interactants can be evaluated. For example, a microphysiorneter can be used to detect the interaction of a compound with a cancer marker without the labeling of either the compound or the cancer marker (McConnell et al. Science 257:1906-1912 [1992]). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and cancer markers.

In yet another embodiment, a cell-free assay is provided in which a cancer marker protein or biologically active portion thereof is contacted with a test compound and the ability of the test compound to bind to the cancer marker protein, mRNA, or biologically active portion thereof is evaluated. Preferred biologically active portions of the cancer marker proteins or mRNA to be used in assays of the present invention include fragments that participate in interactions with substrates or other proteins, e.g., fragments with high surface probability scores.

Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FRET) (see, for example, Lakowicz et al., U.S. Pat. No. 5,631,169; Stavrianopoulos et al., U.S. Pat. No. 4,968,103; each of which is herein incorporated by reference). A fluorophore label is selected such that a first donor molecule's emitted fluorescent energy will be absorbed by a fluorescent label on a second, ‘acceptor’ molecule, which in turn is able to fluoresce due to the absorbed energy.

Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label should be maximal. A FRET binding event can be conveniently measured through standard fluorometric detection means well known in the art (e.g., using a fluorimeter).

In another embodiment, determining the ability of the cancer marker protein or mRNA to bind to a target molecule can be accomplished using real-time Biomolecular Interaction Analysis (BIA) (see, e.g., Sjolander and Urbaniczky, Anal. Chem. 63:2338-2345 [1991] and Szabo et al. Curr. Opin. Struct. Biol. 5:699-705 [1995]). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

In one embodiment, the target gene product or the test substance is anchored onto a solid phase. The target gene product/test compound complexes anchored on the solid phase can be detected at the end of the reaction. Preferably, the target gene product can be anchored onto a solid surface, and the test compound, (which is not anchored), can be labeled, either directly or indirectly, with detectable labels discussed herein.

It may be desirable to immobilize cancer markers, an anti-cancer marker antibody or its target molecule to facilitate separation of complexed from non-complexed forms of one or both of the proteins, as well as to accommodate automation of the assay. Binding of a test compound to a cancer marker protein, or interaction of a cancer marker protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase-cancer marker fusion proteins or glutathione-S-transferase/target fusion proteins can be adsorbed onto glutathione Sepharose beads (Sigma Chemical, St. Louis, Mo.) or glutathione-derivatized microtiter plates, which are then combined with the test compound or the test compound and either the non-adsorbed target protein or cancer marker protein, and the mixture incubated under conditions conducive for complex formation (e.g., at physiological conditions for salt and pH). Following incubation, the beads or microtiter plate wells are washed to remove any unbound components, the matrix immobilized in the case of beads, complex determined either directly or indirectly, for example, as described above.

Alternatively, the complexes can be dissociated from the matrix, and the level of cancer markers binding or activity determined using standard techniques. Other techniques for immobilizing either cancer markers protein or a target molecule on matrices include using conjugation of biotin and streptavidin. Biotinylated cancer marker protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-IgG antibody).

This assay is performed utilizing antibodies reactive with cancer marker protein or target molecules but which do not interfere with binding of the cancer markers protein to its target molecule. Such antibodies can be derivatized to the wells of the plate, and unbound target or cancer markers protein trapped in the wells by antibody conjugation. Methods for detecting such complexes, in addition to those described above for the GST-immobilized complexes, include immunodetection of complexes using antibodies reactive with the cancer marker protein or target molecule, as well as enzyme-linked assays which rely on detecting an enzymatic activity associated with the cancer marker protein or target molecule.

Alternatively, cell free assays can be conducted in a liquid phase. In such an assay, the reaction products are separated from unreacted components, by any of a number of standard techniques, including, but not limited to: differential centrifugation (see, for example, Rivas and Minton, Trends Biochem Sci 18:284-7 [1993]); chromatography (gel filtration chromatography, ion-exchange chromatography); electrophoresis (see, e.g., Ausubel et al., eds. Current Protocols in Molecular Biology 1999, J. Wiley: New York.); and immunoprecipitation (see, for example, Ausubel et al., eds. Current Protocols in Molecular Biology 1999, J. Wiley: New York). Such resins and chromatographic techniques are known to one skilled in the art (See e.g., Heegaard J. Mol. Recognit 11:141-8 [1998]; Hageand Tweed J. Chromatogr. Biomed. Sci. Appl 699:499-525 [1997]). Further, fluorescence energy transfer may also be conveniently utilized, as described herein, to detect binding without further purification of the complex from solution.

The assay can include contacting the cancer markers protein, mRNA, or biologically active portion thereof with a known compound that binds the cancer marker to form an assay mixture, contacting the assay mixture with a test compound, and determining the ability of the test compound to interact with a cancer marker protein or mRNA, wherein determining the ability of the test compound to interact with a cancer marker protein or mRNA includes determining the ability of the test compound to preferentially bind to cancer markers or biologically active portion thereof, or to modulate the activity of a target molecule, as compared to the known compound.

To the extent that cancer markers can, in vivo, interact with one or more cellular or extracellular macromolecules, such as proteins, inhibitors of such an interaction are useful. A homogeneous assay can be used can be used to identify inhibitors.

For example, a preformed complex of the target gene product and the interactive cellular or extracellular binding partner product is prepared such that either the target gene products or their binding partners are labeled, but the signal generated by the label is quenched due to complex formation (see, e.g., U.S. Pat. No. 4,109,496, herein incorporated by reference, that utilizes this approach for immunoassays). The addition of a test substance that competes with and displaces one of the species from the preformed complex will result in the generation of a signal above background. In this way, test substances that disrupt target gene product-binding partner interaction can be identified. Alternatively, cancer markers protein can be used as a “bait protein” in a two-hybrid assay or three-hybrid assay (see, e.g., U.S. Pat. No. 5,283,317; Zervos et al., Cell 72:223-232 [1993]; Madura et al., J. Biol. Chem. 268.12046-12054 [1993]; Bartel et al., Biotechniques 14:920-924 [1993]; Iwabuchi et al., Oncogene 8:1693-1696 [1993]; and Brent WO 94/10300; each of which is herein incorporated by reference), to identify other proteins, that bind to or interact with cancer markers (“cancer marker-binding proteins” or “cancer marker-bp”) and are involved in cancer marker activity. Such cancer marker-bps can be activators or inhibitors of signals by the cancer marker proteins or targets as, for example, downstream elements of a cancer markers-mediated signaling pathway.

Modulators of cancer markers expression can also be identified. For example, a cell or cell free mixture is contacted with a candidate compound and the expression of cancer marker mRNA or protein evaluated relative to the level of expression of cancer marker mRNA or protein in the absence of the candidate compound. When expression of cancer marker mRNA or protein is greater in the presence of the candidate compound than in its absence, the candidate compound is identified as a stimulator of cancer marker mRNA or protein expression. Alternatively, when expression of cancer marker mRNA or protein is less (i.e., statistically significantly less) in the presence of the candidate compound than in its absence, the candidate compound is identified as an inhibitor of cancer marker mRNA or protein expression. The level of cancer markers mRNA or protein expression can be determined by methods described herein for detecting cancer markers mRNA or protein.

A modulating agent can be identified using a cell-based or a cell free assay, and the ability of the agent to modulate the activity of a cancer markers protein can be confirmed in vivo, e.g., in an animal such as an animal model for a disease (e.g., an animal with lung cancer or metastatic lung cancer; or an animal harboring a xenograft of a lung cancer from an animal (e.g., human) or cells from a cancer resulting from metastasis of a lung cancer (e.g., to a lymph node, bone, or liver), or cells from a lung cancer cell line.

This invention further pertains to novel agents identified by the above-described screening assays (See e.g., below description of cancer therapies). Accordingly, it is within the scope of this invention to further use an agent identified as described herein (e.g., a cancer marker modulating agent, an antisense cancer marker nucleic acid molecule, a siRNA molecule, a cancer marker specific antibody, or a cancer marker-binding partner) in an appropriate animal model (such as those described herein) to determine the efficacy, toxicity, side effects, or mechanism of action, of treatment with such an agent. Furthermore, novel agents identified by the above-described screening assays can be, e.g., used for treatments as described herein.

### V. Transgenic Animals

The present invention contemplates the generation of transgenic animals comprising an exogenous cancer marker gene (e.g., gene fusion) of the present invention or mutants and variants thereof (e.g., truncations or single nucleotide polymorphisms). In preferred embodiments, the transgenic animal displays an altered phenotype (e.g., increased or decreased presence of markers) as compared to wild-type animals. Methods for analyzing the presence or absence of such phenotypes include but are not limited to, those disclosed herein. In some preferred embodiments, the transgenic animals further display an increased or decreased growth of tumors or evidence of cancer.

The transgenic animals of the present invention find use in drug (e.g., cancer therapy) screens. In some embodiments, test compounds (e.g., a drug that is suspected of being useful to treat cancer) and control compounds (e.g., a placebo) are administered to the transgenic animals and the control animals and the effects evaluated.

The transgenic animals can be generated via a variety of methods. In some embodiments, embryonal cells at various developmental stages are used to introduce transgenes for the production of transgenic animals. Different methods are used depending on the stage of development of the embryonal cell. The zygote is the best target for micro-injection. In the mouse, the male pronucleus reaches the size of approximately 20 micrometers in diameter that allows reproducible injection of 1-2 picoliters (pl) of DNA solution. The use of zygotes as a target for gene transfer has a major advantage in that in most cases the injected DNA will be incorporated into the host genome before the first cleavage (Brinster et al., Proc. Natl. Acad. Sci. USA 82:4438-4442 [1985]). As a consequence, all cells of the transgenic non-human animal will carry the incorporated transgene. This will in general also be reflected in the efficient transmission of the transgene to offspring of the founder since 50% of the germ cells will harbor the transgene. U.S. Pat. No. 4,873,191 describes a method for the micro-injection of zygotes; the disclosure of this patent is incorporated herein in its entirety.

In other embodiments, retroviral infection is used to introduce transgenes into a non-human animal. In some embodiments, the retroviral vector is utilized to transfect oocytes by injecting the retroviral vector into the perivitelline space of the oocyte (U.S. Pat. No. 6,080,912, incorporated herein by reference). In other embodiments, the developing non-human embryo can be cultured in vitro to the blastocyst stage. During this time, the blastomeres can be targets for retroviral infection (Janenich, Proc. Natl. Acad. Sci. USA 73:1260 [1976]). Efficient infection of the blastomeres is obtained by enzymatic treatment to remove the zona pellucida (Hogan et al., in Manipulating the Mouse Embryo, Cold Spring Harbor Laboratory Press, Cold Spring Harbor, N.Y. [1986]). The viral vector system used to introduce the transgene is typically a replication-defective retrovirus carrying the transgene (Jahner et al., Proc. Natl. Acad Sci. USA 82:6927 [1985]). Transfection is easily and efficiently obtained by culturing the blastomeres on a monolayer of virus-producing cells (Stewart, et al., EMBO J., 6:383 [1987]). Alternatively, infection can be performed at a later stage. Virus or virus-producing cells can be injected into the blastocoele (Jahner et al., Nature 298:623 [1982]). Most of the founders will be mosaic for the transgene since incorporation occurs only in a subset of cells that form the transgenic animal. Further, the founder may contain various retroviral insertions of the transgene at different positions in the genome that generally will segregate in the offspring. In addition, it is also possible to introduce transgenes into the germline, albeit with low efficiency, by intrauterine retroviral infection of the midgestation embryo (Jahner et al., supra [1982]). Additional means of using retroviruses or retroviral vectors to create transgenic animals known to the art involve the micro-injection of retroviral particles or mitomycin C-treated cells producing retrovirus into the perivitelline space of fertilized eggs or early embryos (PCT International Application WO 90/08832 [1990], and Haskell and Bowen, Mol. Reprod. Dev., 40:386 [1995]).

In other embodiments, the transgene is introduced into embryonic stem cells and the transfected stem cells are utilized to form an embryo. ES cells are obtained by culturing pre-implantation embryos in vitro under appropriate conditions (Evans et al., Nature 292:154 [1981]; Bradley et al., Nature 309:255 [1984]; Gossler et al., Proc. Acad. Sci. USA 83:9065 [1986]; and Robertson et al., Nature 322:445 [1986]). Transgenes can be efficiently introduced into the ES cells by DNA transfection by a variety of methods known to the art including calcium phosphate co-precipitation, protoplast or spheroplast fusion, lipofection and DEAE-dextran-mediated transfection. Transgenes may also be introduced into ES cells by retrovirus-mediated transduction or by micro-injection. Such transfected ES cells can thereafter colonize an embryo following their introduction into the blastocoel of a blastocyst-stage embryo and contribute to the germ line of the resulting chimeric animal (for review, See, Jaenisch, Science 240:1468 [1988]). Prior to the introduction of transfected ES cells into the blastocoel, the transfected ES cells may be subjected to various selection protocols to enrich for ES cells which have integrated the transgene assuming that the transgene provides a means for such selection. Alternatively, the polymerase chain reaction may be used to screen for ES cells that have integrated the transgene. This technique obviates the need for growth of the transfected ES cells under appropriate selective conditions prior to transfer into the blastocoel.

In still other embodiments, homologous recombination is utilized to knock-out gene function or create deletion mutants (e.g., truncation mutants). Methods for homologous recombination are described in U.S. Pat. No. 5,614,396, incorporated herein by reference.

### Experimental

The following examples are provided in order to demonstrate and further illustrate certain preferred embodiments and aspects of the present invention and are not to be construed as limiting the scope thereof.

### EXAMPLE 1

### Methods

### Sequence and Domain Analysis

3,068,965 mRNA sequences were extracted from GenBank and mapped to the human genome by BLAT. Sequences that aligned to exon boundaries of two different genes were considered fusion chimeras and compared to the Mitelman database of known fusions to identify deposited fusion sequences. The fusion proteins were delineated based on the exon recombination sites and the open reading frames (ORF) of both partners. The conserved domains in each fusion protein were delineated based on the protein-domain mapping data extracted from the Entrez Gene database.

Interrogation of the Gene-Fusion Network with the Molecular-Interaction Network

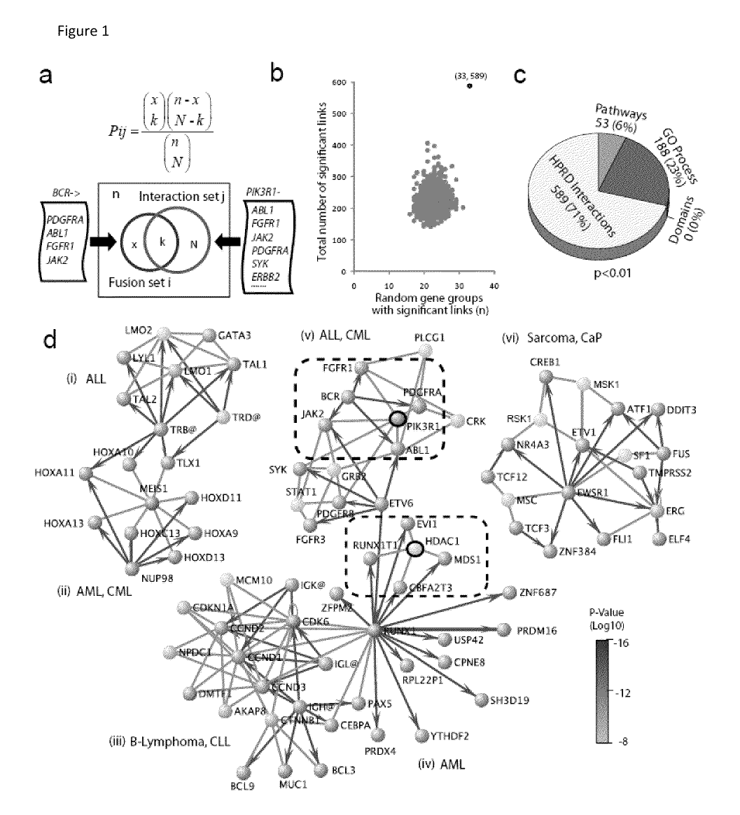

The molecular interactions for human genes were extracted from the HPRD database (Vastrik, Genome Biol 8:R39 2007), a resource that contains expert curated reference protein-protein interactions. The gene fusion network was constructed using established fusions from the Mitelman database (Mullighan, Nature 446:758 2007). Hypergeometric probabilities were applied to detect the enrichment of gene fusion partners in the molecular interactions sets. Suppose an interaction gene set for gene j, consisting of N interacting genes, and a fusion partner set for gene i, consisting of x partners; the intersection of these two sets is calculated as kij. Then, taking the complete set of all human genes (size n), the probability that kij is a more significant overlap than expected by chance is calculated using the hypergeometric distribution (FIG. 1a). Using these statistics, the gene fusion network was interrogated with the molecular interaction network, which yielded 589 significant shared interacting genes for 30 out of 90 fusion gene groups. To evaluate the top FIN hub candidates, the fusion-interaction (FI) network was resolved for shared interacting genes with p value less than 10⁻⁷ (≧3 connectivities with a fusion partner group). The FI network was visualized by VisANT⁶, and then processed by the spring embedded relax function. The fusion partner groups that fall into the six major clusters were exhibited together with their shared interacting genes on FIG. 1d. The hubs were nominated based on the significance from the above statistical test within each subset of connected fusions, and ablating drugs were identified by mapping the hubs to the DrugBank database as of Aug. 8, 2008 (Wishart, Nucleic Acids Res. 34:668 2006).

### Enrichment Analysis of Cancer Genes in the Compendium of Molecular Concepts and Calculation of the ConSig-Score

28,963 molecular concepts were compiled from the Gene Ontology database, the Reactome database, the Kyoto Encyclopedia of Genes and Genomes (KEGG), Biocarta, the HPRD database, and the Entrez Gene conserved domain database (Table 1). In the processing of gene ontologies, the genes that appeared in the child ontologies were subtracted from the parents to avoid duplicate representation. Next, the enrichment of established fusion or point mutation genes was mapped and analyzed against all concepts and the fusion and mutation ConSig-scores were calculated for all known human genes based on their participation in signature concepts. The point mutation genes were compiled from the Cancer Gene Census. Computationally, let k be the number of concepts associated with a specified gene. Let ni represent the number of total genes and xi represent the number of fusion or mutation genes participating in a given concept i, i=1, . . . , k. The ConSig-score then integrates a signal measure of fusion or mutation genes participating in concept i (xi/ni 0.5) over all possible i, with the incorporation of normalization factor for k using the formula:

$$\frac{1}{\sqrt{k}} {\sum }_{i = 1}^{k} {log}_{20} ( 1 + \frac{{x}_{i}}{\sqrt{{n}_{i}}} )$$

With this computation, if a gene has high probability to be involved in gene fusions or mutations, the fusion/mutation ConSig-score will be high respectively; thus the radius in the two-dimensional ConSig-score plot for fusions and mutations will correlate with the role of tested genes in cancer. To eliminate the bias from the gene itself in the overlap, the seeding genes were subtracted from the signature concepts during the calculation of their own ConSig score.

### Kolmogorov-Smirnov Analysis for ConSig-Score

The established cancer genes from the Mitelman and cancer gene consensus databases were used as a prototype, and compiled into ordered gene lists by descending rConSig-score. The enrichment of these established cancer genes in top scored genes was measured using the Kolmogorov-Smirnov Rank statistic (K-S, Hollander and Wolfe, 1999, p=1.39e-114). Let X be the number of known cancer genes in the ordered gene list (X=470). Set Y=n/X-1 where n represents the total number of human genes interrogated and construct a vector V where V(i) is the component corresponding to gene i. Let V(i)=Y if i is in the target gene set and V(i)=−X if not. Thus, the K-S statistical score is the maximum value of the running sum of consecutive values of V(i).

### Random Gene Set Statistics

Randomization tests were performed to evaluate the statistical significance of the observations. First, to test whether the fusion partner groups are significantly more linked by mutual interacting genes than by chance, randomized gene sets were generated with the same gene sizes and equal amount of interacting genes as the fusion partner groups. Fusion genes that have less than 58 interacting genes are substituted by genes with the same amount of interactions; the others are substituted randomly by genes having equal to or more than 58 interactions. Then the number of statistically significant links generated by HPRD database were calculated (p<0.01). This process was permutated for 1000 times; none of the random gene family sets generated more significant links than fusion partner groups (p<0.001). Second, to test the significance of ConSig score in isolating known cancer genes, randomized gene sets were generated corresponding to the sizes of the fusion and mutation gene lists. Then ConSig-score was calculated as if these random genes were actual cancer genes. As above, the K-S score was calculated and recorded. This process was repeated 10 times for each cancer gene list size, resulting in non-significant K-S statistical scores, thus validating the K-S score as unbiased and providing a null distribution of ConSig-score under the null hypothesis of no functional signal in the input gene list.

### Meta-Analysis of Public Array CGH/SNP Datasets for Multiple Human Cancers

Public array CGH/SNP datasets were complied from Gene Expression Omnibus. A total of seven datasets were included in this study (GSE4659, GSE8918, GSE7255, GSE9611, GSE9113, GSE3930 and GSE8398), covering six cancer types (leukemia, lymphoma, sarcoma, salivary adenoma, brain and prostate tumors). The samples from each dataset were manually curated and classified according to pathological associations. For Affymetrix SNP arrays, model-based expression was performed to summarize signal intensities for each probe set, using the perfect-match/mismatch (PM/MM) model. For copy number inference, raw copy numbers were calculated for each tumor sample by comparing the signal intensity of each SNP probe set against a diploid reference set of samples. In two channel array CGH datasets, the differential ratio between the processed testing channel signal and processed reference channel signal was calculated. All resulting relative DNA copy number data were log2 transformed, which reflects the DNA copy number difference between the testing and reference channels. For normalization, log ratios were transformed into a normal distribution with a mean of 0 under the null model assumption. The data were then segmented by the circular binary segmentation (CBS) algorithm (Olshen et al., Biostat. 5: 557 2004). Cutoffs of 0.3 and −0.4 were used to call amplifications and deletions, respectively. To explore the evidence of fusion breakpoint pattern at the NFE2 loci in lung cancer, the SNP array data of lung cancer tissues and cell lines from publication 24 and array express (E-MTAB-38), respectively were compiled. The relative copy number data were inferred and segmented as discussed above to reveal the DNA breakpoint patterns.

### Analysis of Paired-End Transcriptome Sequencing Data

Mate pair transcriptome reads were mapped to the human genome (hg18) and Refseq transcripts, allowing up to two mismatches, using Efficient Alignment of Nucleotide Databases (ELAND) program within the Illumina Genome Analyzer Pipeline. Using a Perl script, the Illumina export output files were parsed to identify chimerical mate-pairs, with the following criteria: (a) putative chimeras must be supported by at least one mate pair that is best unique match across genome; and at least three mate pairs in total; (b) the distances between the 5′ and 3′ partners of the intra-chromosome chimeras must be more than 1 Mb. The resultant candidate chimeras were aligned by rConSig-score of 3′ partner genes to reveal functionally important gene fusions in lung cancer cell lines.

### Reverse Transcription PCR and Sequencing

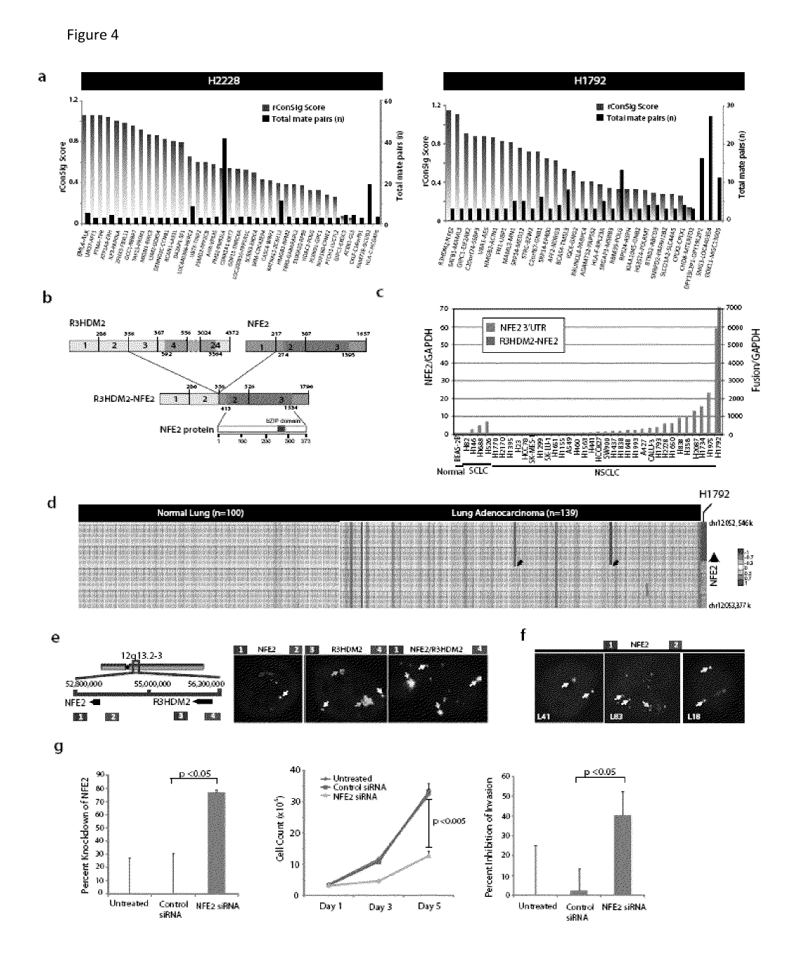

RNAs from lung cancer cell lines, obtained from the American Type Culture Collection (Manassas, Va.), were extracted and reverse transcribed with superscript III (Invitrogen, Carlsbad, Calif.) and random primers. Polymerase chain reaction was performed with Platinum Taq High Fidelity and fusion or NFE2 specific primers for 35 cycles. The primers used in this study are listed in Table 5. Products were resolved by electrophoresis on 1.5% agarose gels, and TOPO TA cloned into pCR 4-TOPO. Purified plasmid DNA from at least 4 colonies was sequenced bi-directionally using M13 Reverse and M13 Forward primers on an ABI Model 3730 automated sequencer at the University of Michigan DNA Sequencing Core. Quantitative PCR (qPCR) was performed using the StepOne Real Time PCR system (Applied Biosystems, Foster City, Calif.). The amount of each target gene relative to the housekeeping gene glyceraldehyde-3-phosphate dehydrogenase (GAPDH) for each sample was determined using the comparative threshold cycle (Ct) method (Applied Biosystems User Bulletin #2). For the experiments presented in FIG. 4c, the relative amount of the target gene was calibrated to the relative amount from a lung cancer cell line with the latest Ct value.

### Gene Expression Data Analysis

To determine the expression of R3HDM2 and NFE2 in lung cancer cell lines and normal tissues, the Richard Wooster et al gene expression study of 73 lung cancer cell lines, and Richard B. Roth et al 40 normal tissue dataset (Neurogenetics 7:67 2006) were interogated using the Oncomine database (Rhodes, Neoplasia 9:166 2007). Descriptions of tissue types from the datasets are provided in Table 13.

Fluorescence in situ Hybridization (FISH)

To detect possible translocations on lung cancer cell lines involving R3HDM2 and NFE2 loci, break-apart and colocalizing probe FISH strategies, with two probes spanning the R3HDM2 locus (digoxin-dUTP labeled BAC clone RP11-258J5 (5′ R3HDM2) and biotin-14-dCTP labeled BAC clone RP11-799O6 (3′ R3HDM2)) and NFE2 locus (digoxin-dUTP labeled BAC clone RP11-753H16 (5′ NFE2) and biotin-14-dCTP labeled BAC clone RP11-621J12 (3′ NFE2)) were used. All BAC clones were obtained from the Children's Hospital of Oakland Research Institute (CHORI). Prior to FISH analysis, the integrity and purity of all probes were verified by hybridization to metaphase spreads of normal peripheral lymphocytes. For interphase FISH on lung cancer cell lines, interphase spreads were prepared using standard cytogenetic techniques. For interphase FISH on a lung cancer tissue microarray, tissue hybridization, washing and color detection were performed as described (Rubin Cancer Res 64:3814 2004; Garraway, Nature 436:117 2005). The total evaluable cases include 76 lung adenocarcinoma cases. For evaluation of the interphase FISH on the TMA, an average of 50-100 cells per case were evaluated for assessment of the NFE2 rearrangement. In addition, formalin-fixed paraffin-embedded (FFPE) tissue sections from a positive case were used to confirm the TMA results.

### Small RNA Interference, Cell Proliferation and Invasion Assays

The NFE2 fusion positive H1792 cell line and a H460 cell line with low NFE2 expression were plated into 10 cm dishes, and transfected with siRNA against NFE2 (J-010049-06, Dharmacon, Chicago, Ill., USA) or non-targeting controls. Transfection was performed with oligofectamine following manufacturer's suggestion (Invitrogen). Forty eight hours post-transfection, cells were trypsinized and counted. For each treatment, equal amount of cells were plated into 24-well plates for cell counting, 96-well plates for WST-1 assay, Boyden invasion chambers for invasion assay. The rest cells were harvested for qPCR analysis. The knockdown study on H1792 cell lines was performed twice. Cell counting analysis was performed by Coulter counter (Beckman Coulter, Fullerton, Calif.) at the indicated time points in triplicate. WST-1 proliferation assay was performed using manufacturer's protocol. Invasion assay was performed as described previously (Cao, Oncogene 27:7274 2008).

### Results

Gene fusions resulting from chromosomal rearrangements often define molecular subtypes of cancers and appear as initial events in oncogenesis¹. The discovery of recurrent fusions in common epithelial cancers (Tomlins et al., Science 310:644 2005; Soda, Nature 448:561 2007) has stimulated a widespread search for novel gene fusions. Yet, new fusion discovery and molecular targeting of known fusions is complicated by the complex biological behavior displayed by fusion genes. First, most genes involved in fusions recombine with many different partners, forming interrelated gene fusion networks (Kumar-Sinha et al., Nat. Rev. Cancer 8:497 2008). Second, recurrent gene fusions in carcinomas are often found in the background of many non-specific gene fusions, which illustrates the karyotypic complexity of solid tumor evolution. Distinguishing non-specific (passenger) fusions from recurrent (driver) fusions provides useful information. This study describes the functional and genetic landscape of fusion genes and characterize fundamental principles to help facilitate new gene fusion discovery from large-scale genomic data and next generation sequencing (NGS) data.

### Understanding the Recombination of Fusion Partners

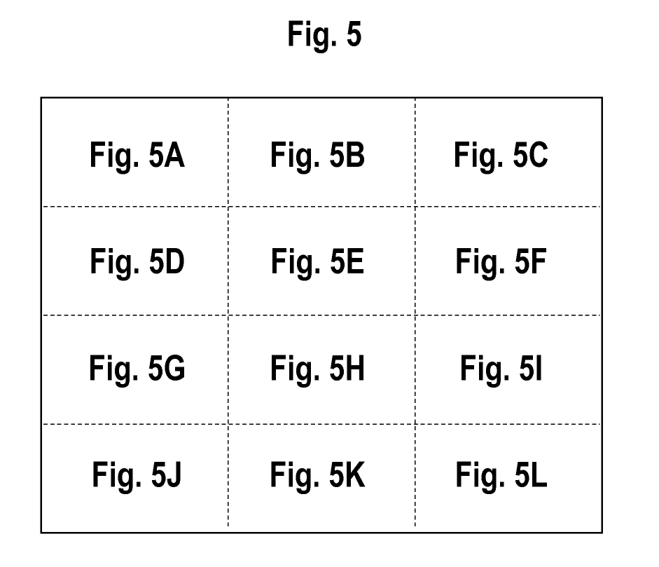

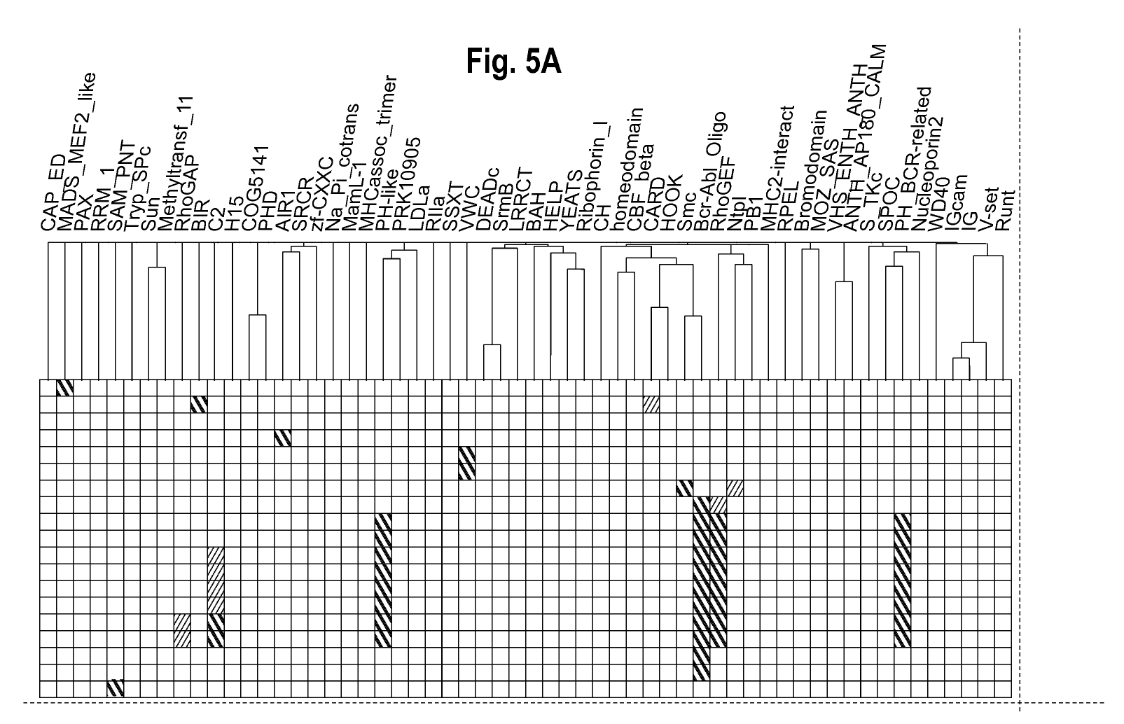

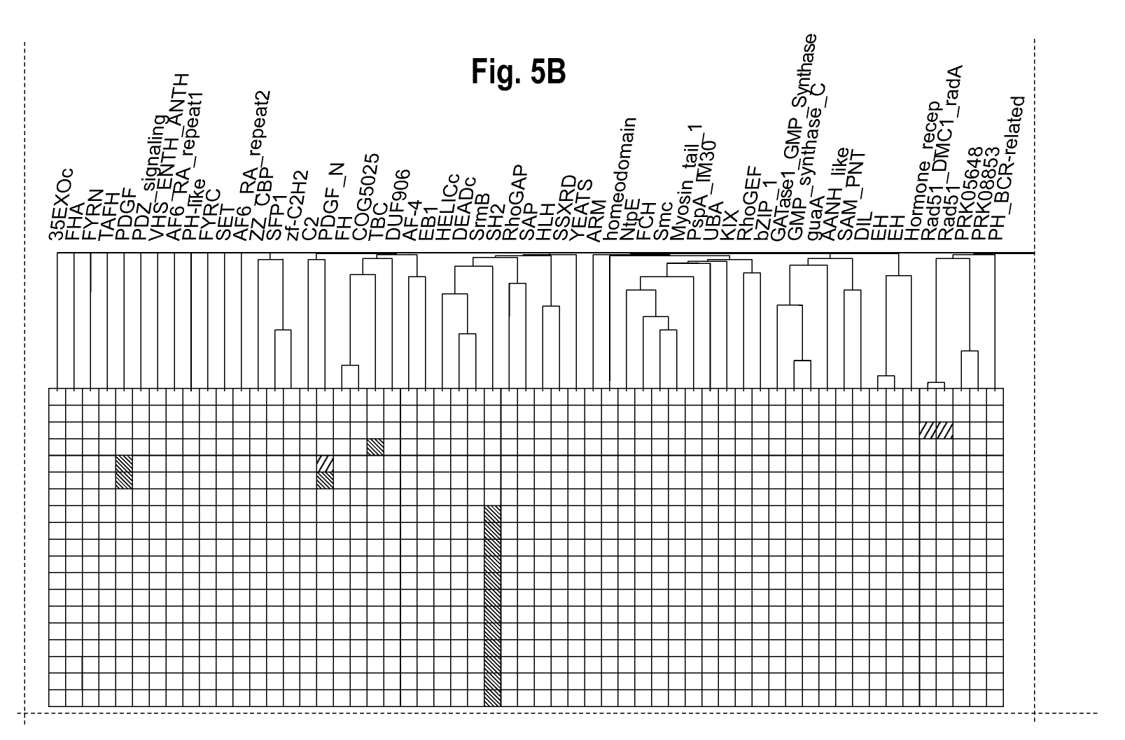

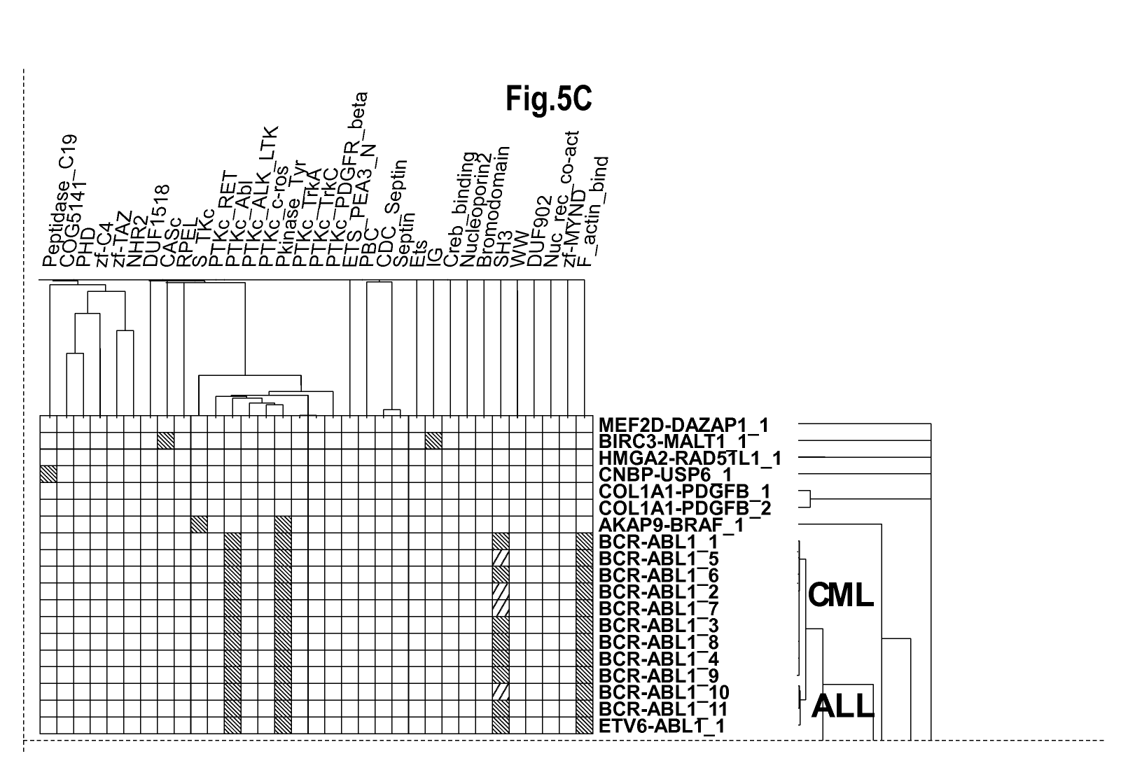

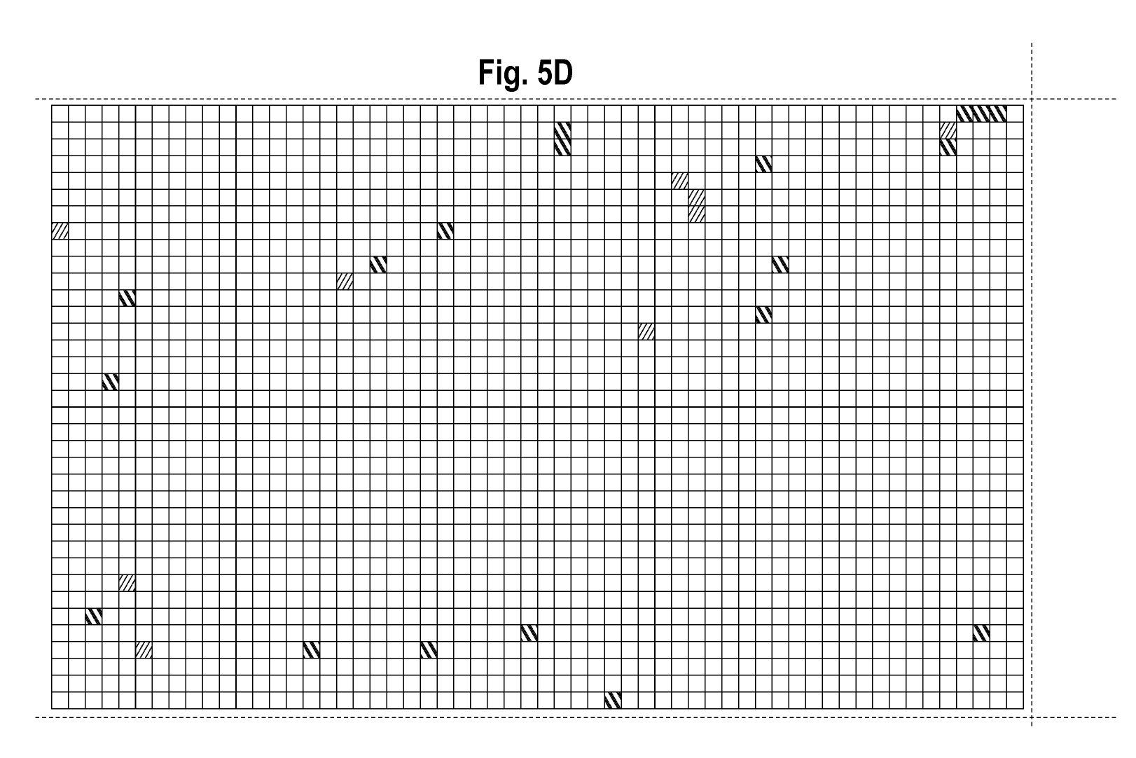

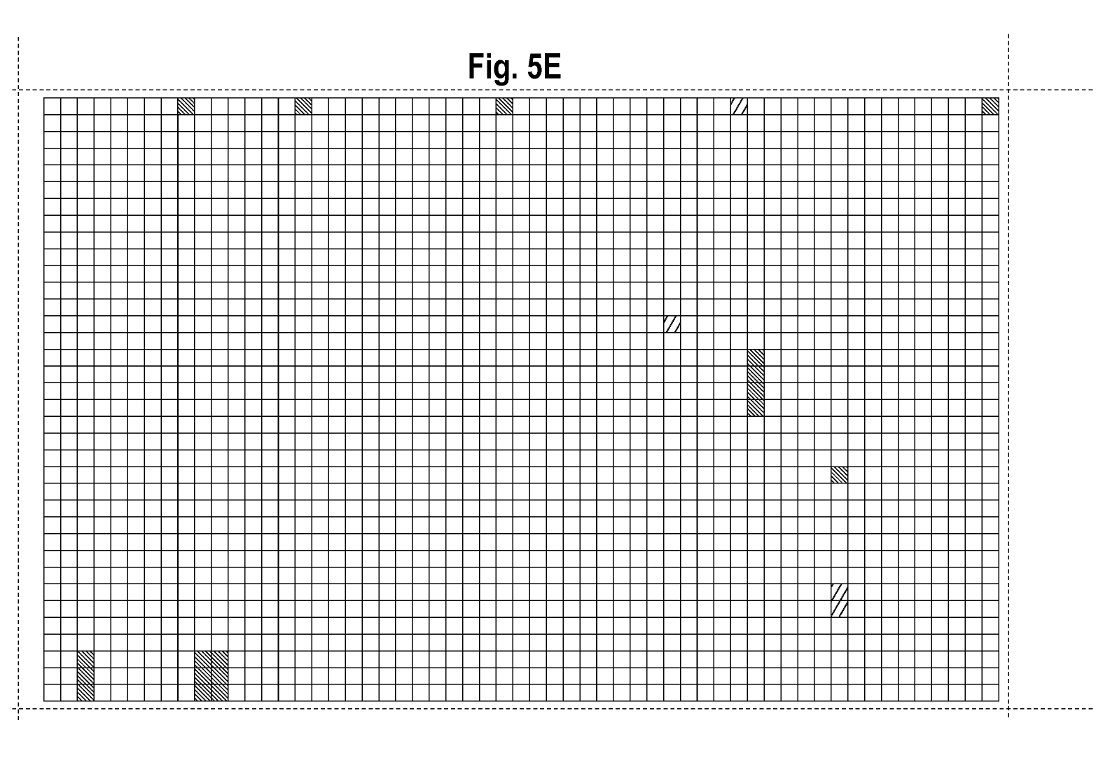

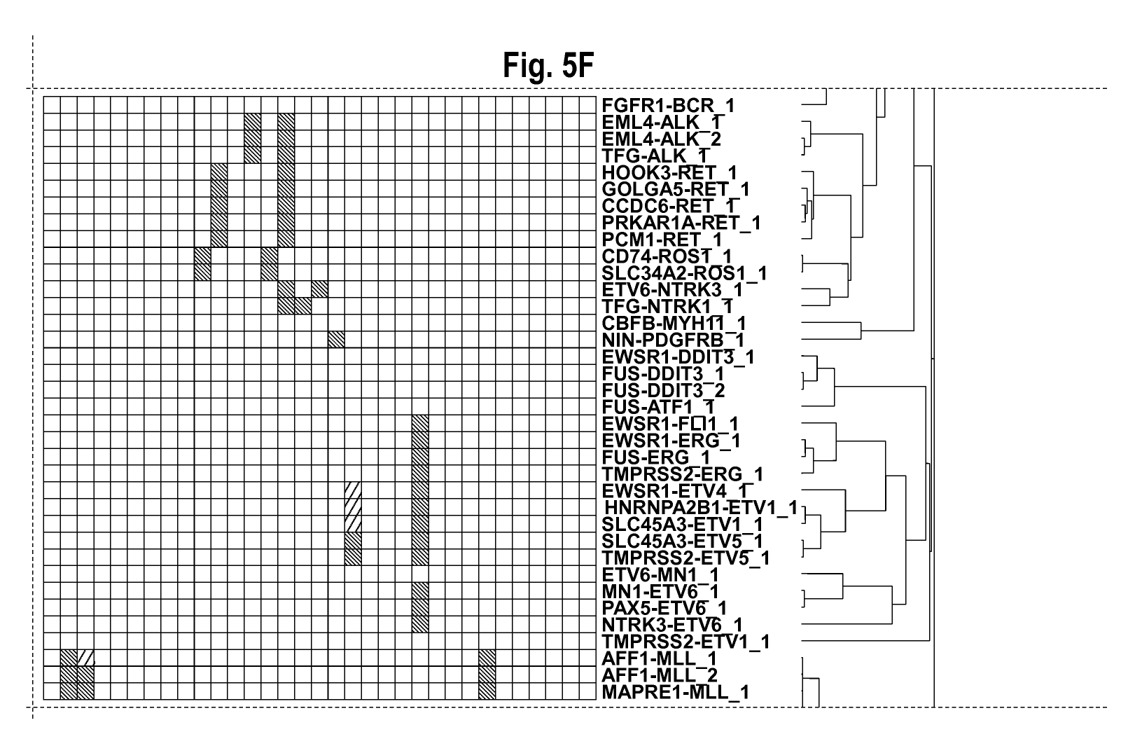

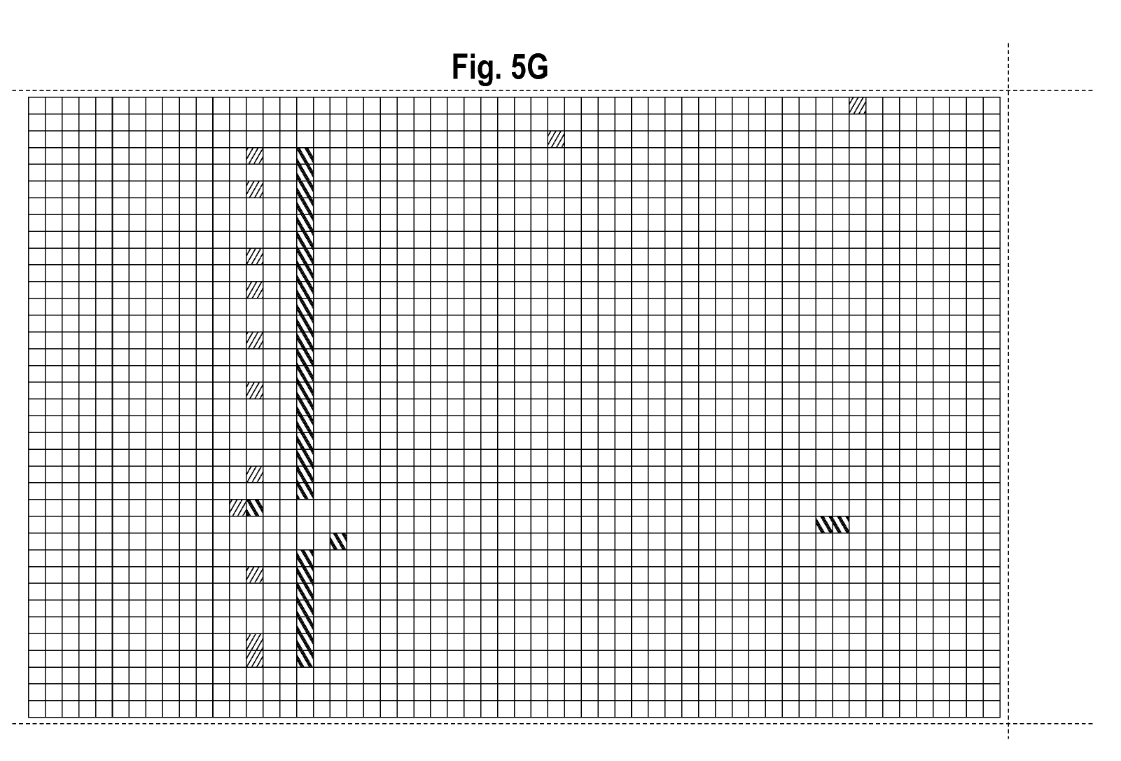

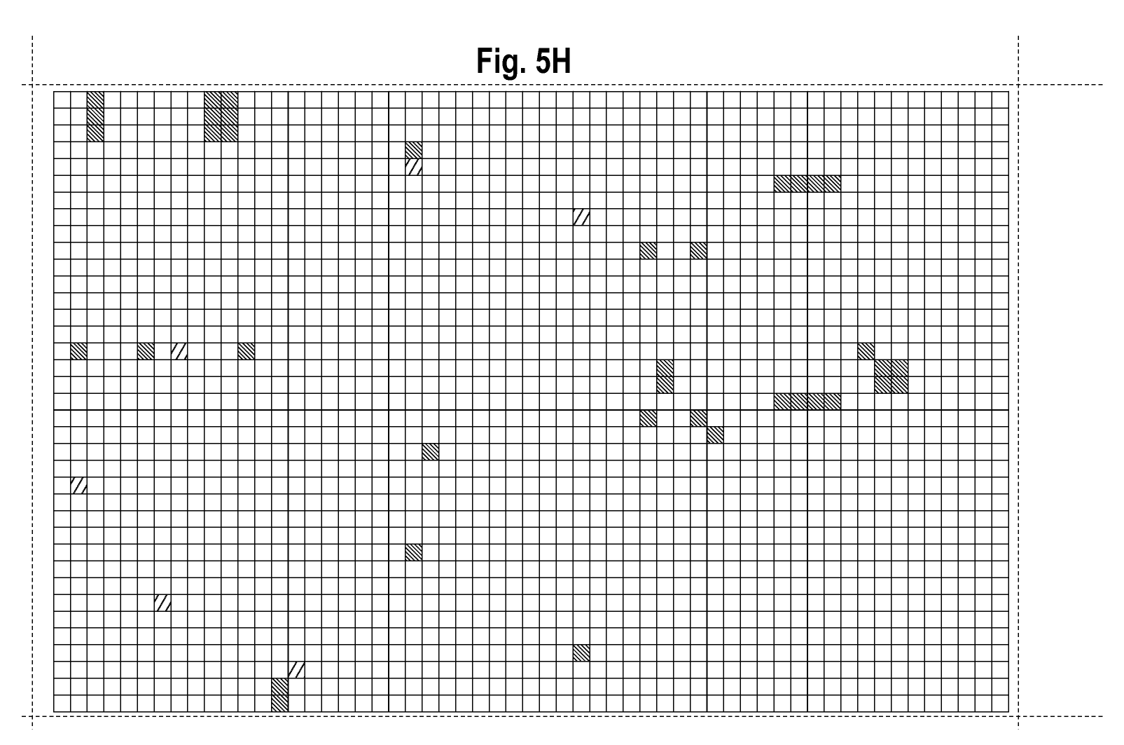

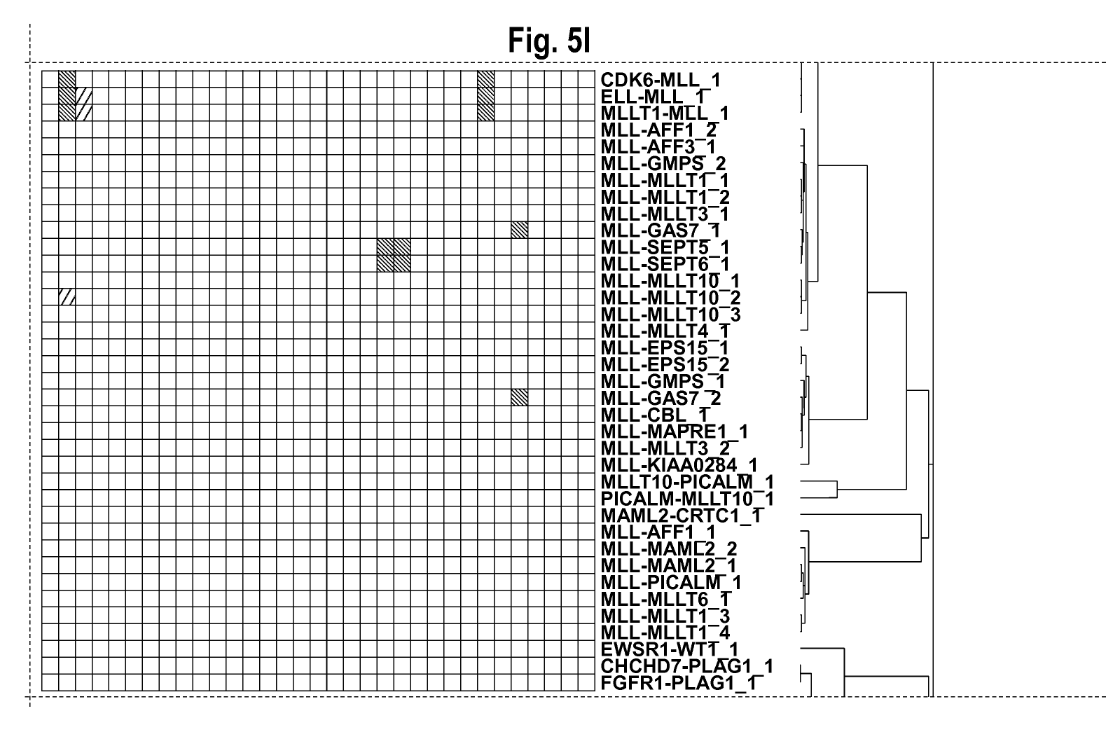

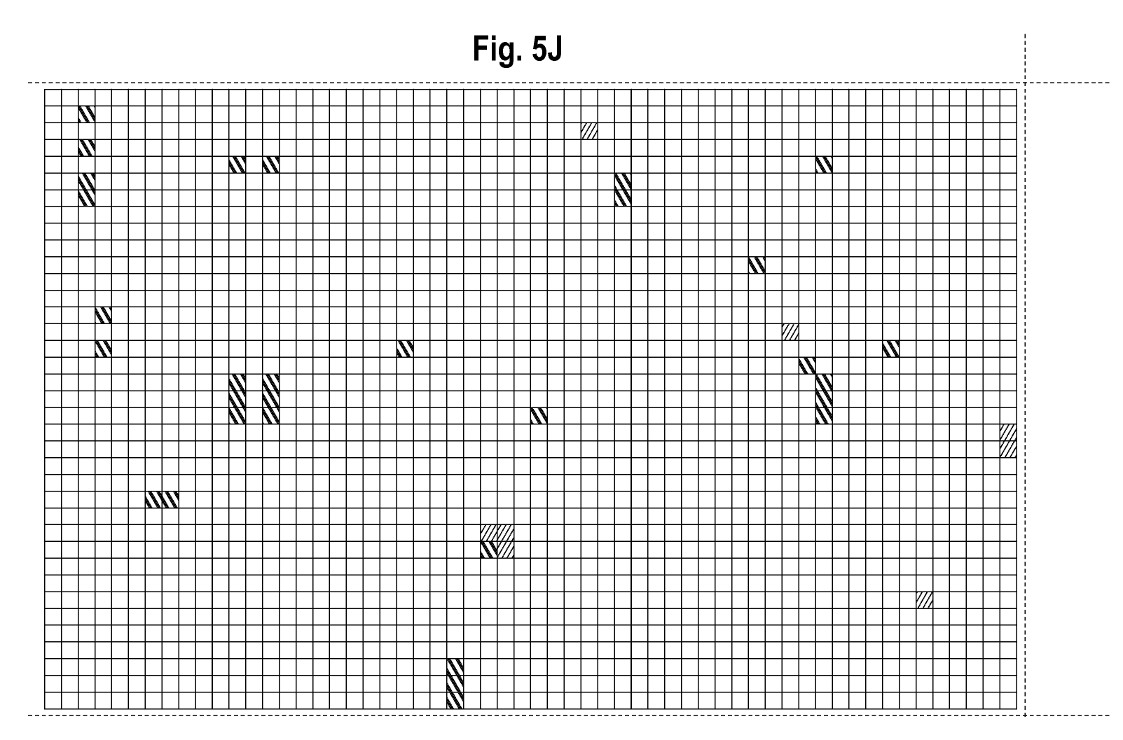

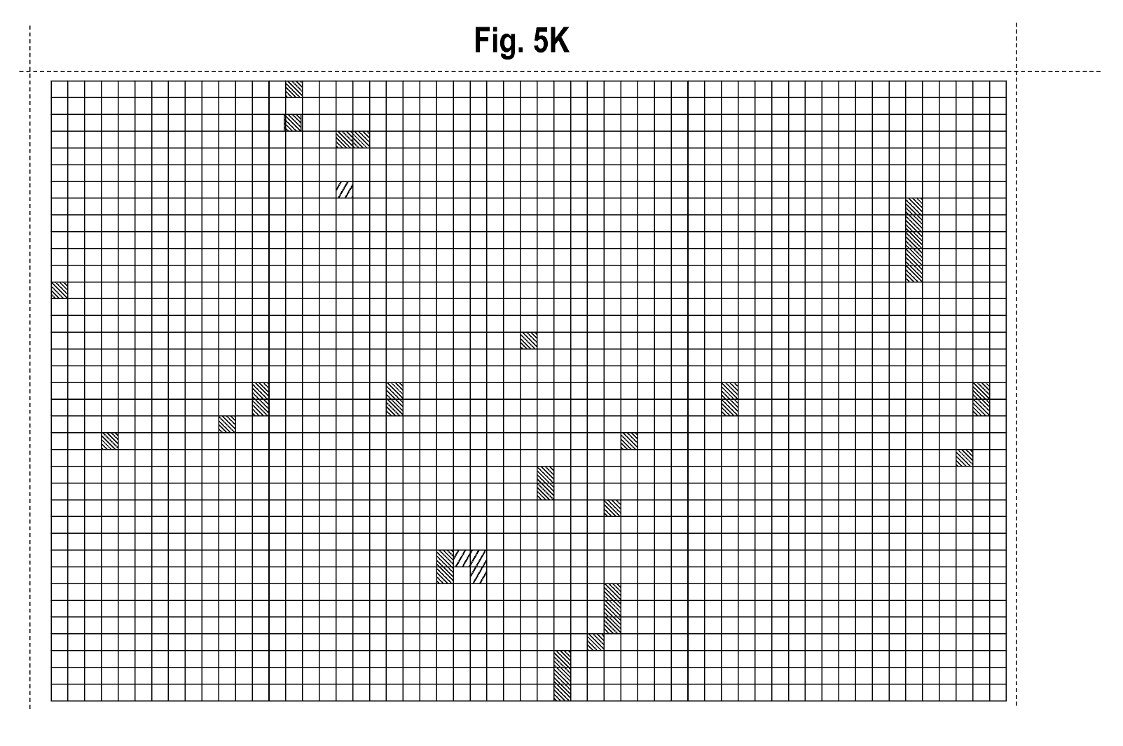

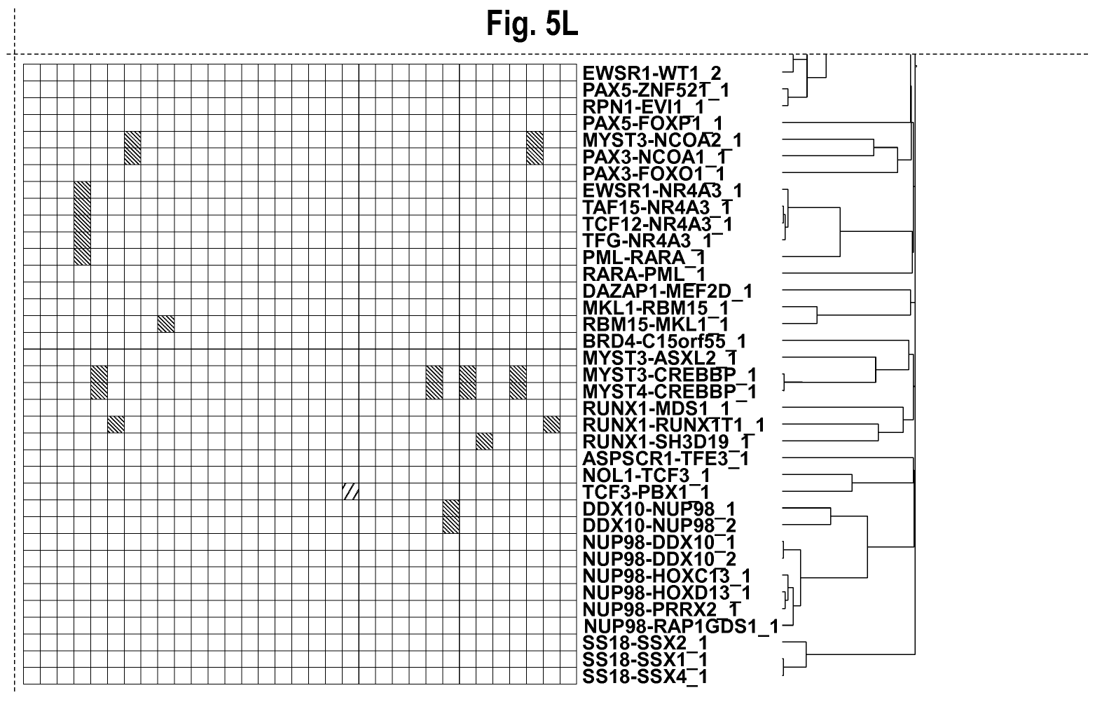

To determine common characteristics of fusion gene recombinations, the hypothesis that fusion genes sharing a common partner might share common domain architectures was investigated. Using Genbank, core nucleotide sequences of chimeras representing known fusions were obtained. Open reading frames and their domain architectures were determined using the Entrez Gene conserved domain database. The resulting unique domain architectures (Table 3) were clustered by domain similarities, enabling the global analysis of domain recombination in gene fusions (FIG. 5). The domain architectures of fusion proteins are very diverse, especially for 5′ partners. In addition, clustering gene fusions according to their domain architectures resulted in few pathologically-related clusters; the majority of the clusters did not show tumor entity specificity. This indicated the existence of other major factors influencing fusion gene recombinations, such as preferential selection for shared pathways or gene ontologies.

The pathway data was then compiled from Reactome, Kyoto Encyclopedia of Genes and Genomes (KEGG), and Biocarta, and shared pathways within fusion partner groups were analyzed. However, most fusion genes with a mutual partner are involved in distinct cell signaling pathways. Yet, because canonical pathways may not encompass the complexities of cell biology, the molecular interaction database was interrogated to generate a comprehensive view of cancer signaling. The 90 fusion partner groups derived from the Mitelman database was mapped with the molecular interaction network extracted from the Human Protein Reference Database (HPRD). For all human genes in the database, the interaction gene set (j) was defined to be all genes that interact with gene j. If one denotes a given fusion gene and its fusion partners as i and (i), respectively, one can then individually test the significance of overlap between every set of fusion partners (i) with every gene interaction set (j) using hypergeometric distribution (FIG. 1a). In aggregate, this analysis yielded a total of 589 genes that shared significant interaction among 33 out of 90 fusion partner groups in the Mitelman database. The top shared interacting genes are supplied in Table 4.

To systematically evaluate the importance of shared interacting genes in fusion gene recombinations, these statistics were applied to the pooled domains, pathways, Gene Ontology (GO) biological process, and HPRD interactions data (see Table 1 for a compendia of molecular concepts used). Each of these categories was benchmarked by statistically assessing the number of molecular concepts shared by fusion partner groups. By setting the p-value threshold to 0.01, the HPRD interactions data yielded 589 unique explanations, while pathway data and GO general process terms gave only 53 and 188, respectively (FIG. 1c).

To explore the functional role of the most significant shared interacting genes more deeply, the fusion-interaction (FI) network was resolved by setting the p value to 10⁻⁷. FI networks were visualized using the VisANT program (Kanehisa et al., Nucleic Acids Res. 32:277 2004), and six major clusters of interactions that connected gene fusions from similar tumor entities were observed (FIG. 1d). The shared interacting genes with the greatest statistical significance in each subset of connected fusions were designated as “fusion-interaction hubs” (FI hub) in each cluster. For example, BCR has four 3′ partners, ABL1, FGFR1, JAK2, and PDGFRA, all of which interact with PIK3R1, a subunit of PI3K (p=9.54×10⁻¹¹). This finding indicated that BCR fusion partners interact with and activate PIK3R1 as part of leukaemogenesis, which was confirmed by mining the literature to validate this hypothesis (Prasad, Nucleic Acids Res. 37:767 2009; Hu, Nucleic Acids Res. 35:625 2007; Chen Endocr. Relat. Cancer 14:513 2007; Chen et al., J. Cell. Biochem. 101:1492 2007; Vantler, FEBS Lett. 580:6769 2006; Walz et al., Leukemia 22:1320 2008). These results show the utility of the FI networks in elucidating fusion biology by distinguishing key genes that serve as network hubs with functional importance in mediating fusion signaling

To test whether fusion genes are significantly enriched for mutual interacting genes, 90 gene sets with an equivalent level of connectivity as the fusion partner groups were randomly chosen (see Methods), and the extent to which they were linked by mutual interacting genes was determined. This process was repeated 1000 times, and then the total number of significant links and the number of gene groups having these links were plotted (FIG. 1b). The number of links generated is significantly greater for fusion genes, validating this observation (p<0.001).

### Quantification of Concept Signatures

The fact that cancer-related fusion partner groups tend to cluster around shared interacting genes or share common gene ontologies led to the development of a method that could filter non-specific gene fusions. It was contemplated that such “signature molecular concepts” frequently found in fusion genes may be used to define biologically meaningful gene fusions underlying cancer, similar to signature genes defining certain phenotypes. This requires a systematic characterization of all fusion genes as a coherent group from multiple functional perspectives.

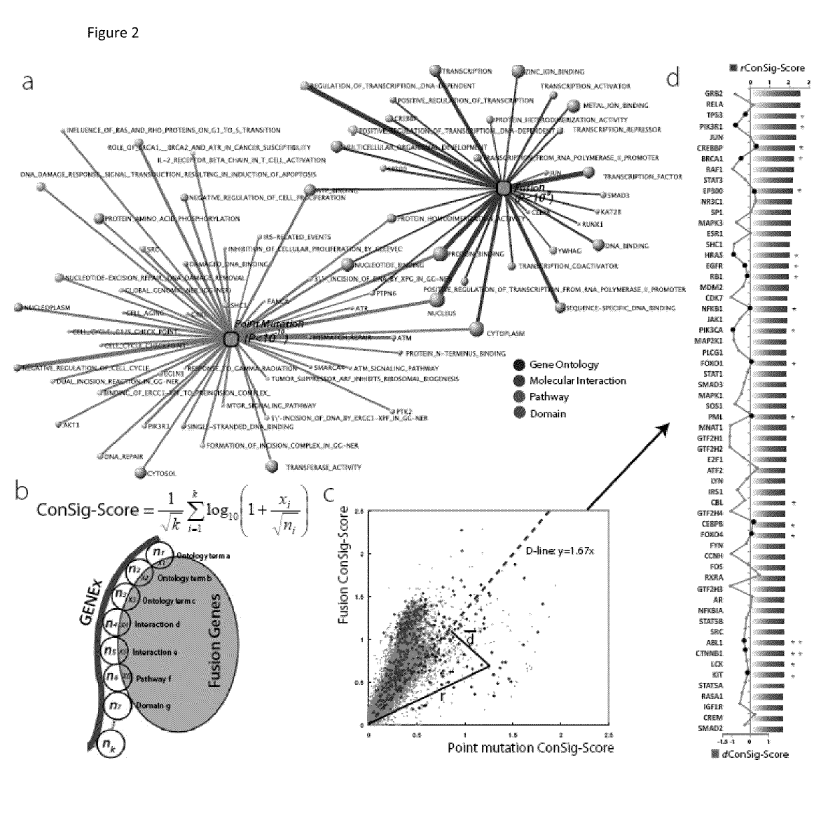

To contrast the functional characteristics of fusion genes, a comparative enrichment analysis was performed using point mutation genes in cancer. Assessing the association of all fusion and mutation genes to the compendium of molecular concepts by Fisher's exact test generated two sets of minimally-overlapping signature concepts (FIG. 2a). In this setting, fusion genes display molecular concepts related to signal transduction and transcription activation; in contrast, mutation genes display molecular concepts related to DNA repair and cell cycle checkpoints. Here, the set of molecular concepts populated by a given gene set was defined as a “concept signature”. Using these concept signatures, it was hypothesized that genes involved in fusions or mutations could be distinguished from each other and from the remaining human genes by algorithmically compiling signature concepts. Toward this end, an algorithm termed Concept Signature score (ConSig-score) was designed to quantify the relevance of genes underlying cancer by the strength of their association with the signature concepts (FIG. 2b). For each gene, the ConSig-score integrates a signal measure of fusion or mutation genes participating in each concept i assigned to this gene, with incorporation of normalization factors for concept size ni and the total number of assigned concepts k.

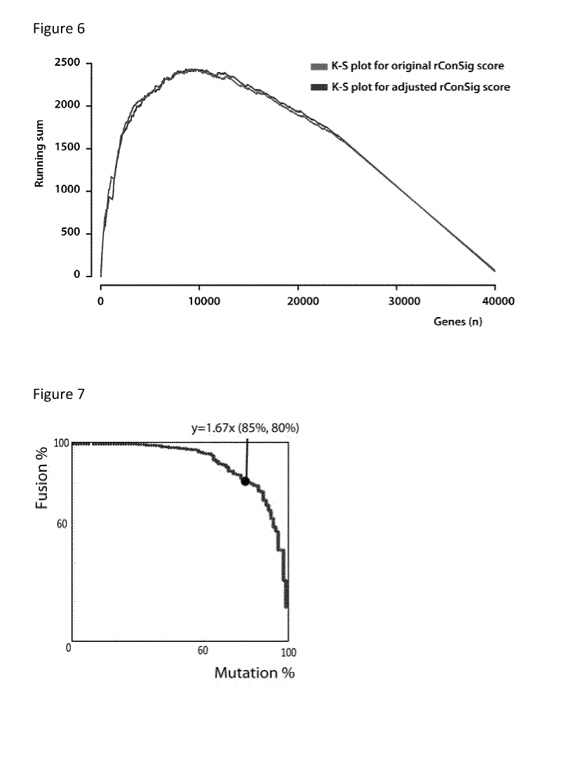

The next step in this analysis was to remove the redundant information from the calculation of the ConSig score. First, to avoid redundant representation in the GO database, the genes that appeared in the child ontologies were subtracted from the parents. Second, to eliminate the bias from the gene itself in the overlap, the seeding genes were subtracted from the signature concepts during the calculation of their own ConSig score. Finally, to minimize the redundant information in the interactome and pathway databases, the pathways significantly overlapping with the molecular interactions (Fisher's exact test, p<0.01) were removed from the calculation of the ConSig score. However, this adjusted ConSig score did not demonstrate an advantage over the initial approach employed (FIG. 6).

Next, all known human genes were tested with the fusion and mutation ConSig analysis. A plot of the fusion vs. mutation ConSig-scores produced segregation of known fusion genes from mutation genes (FIG. 2c). The distinction line (D-line), y=1.67x, was determined by testing optimal separation capacity, which separates 85% of mutation genes from 80% of fusion genes (FIG. 7). This significant segregation indicates the distinct functionality of fusion and mutation genes that occur in cancer. In this setting, the radius to the zero point is defined as the radial ConSig-score of a gene (rConSig-Score), which indicates the strength of association with signature concepts of both fusion and mutation genes, thus implies the functional relevance of candidate genes in cancer. The distance vector from the node to the D-line, which illustrates a distinction between fusion and mutation genes, is defined as the distinction ConSig Score (dConSig-Score). Rating all human genes by the rConSig-Score produced enrichment of established cancer genes in top-scoring genes, with the majority of fusion or mutation genes matching the prediction from the dConSig-Score (FIG. 2d). Replacing the fusion or mutation gene sets with random gene sets produced no enrichment of the randomly selected genes, thus validating the significance of this observation. The ConSig algorithm is able to segregate fusion genes and mutation genes and is useful in the identification of biologically-important gene fusions from NGS data, where a large number of candidate gene fusions hinders a quick evaluation of their functional importance (see FIG. 4).

### Genetic Characteristics of Unbalanced Fusion Genes

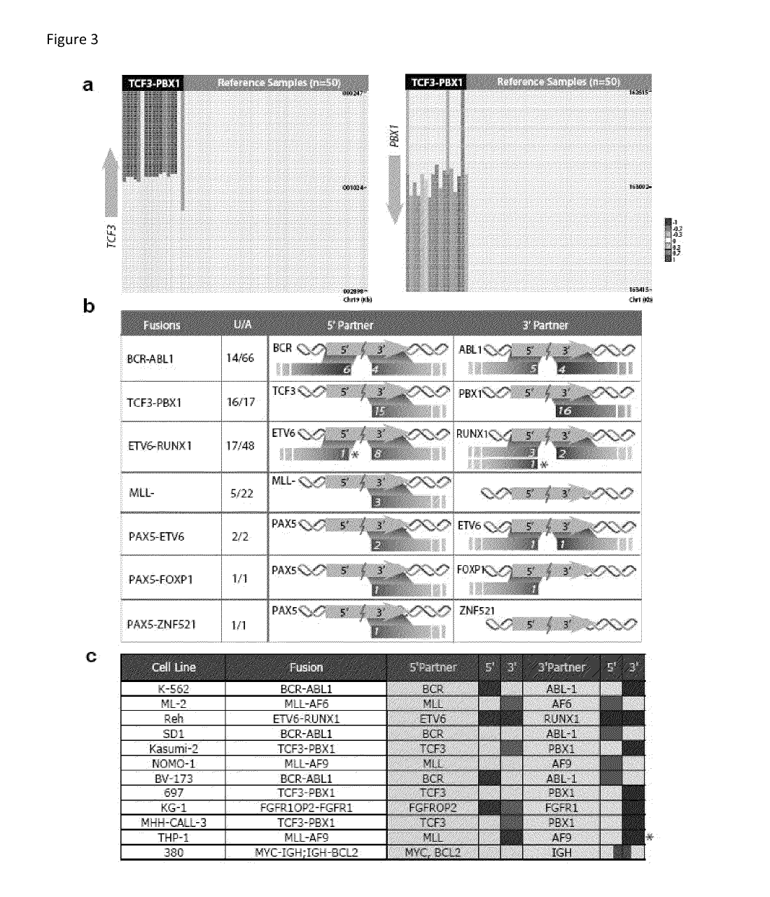

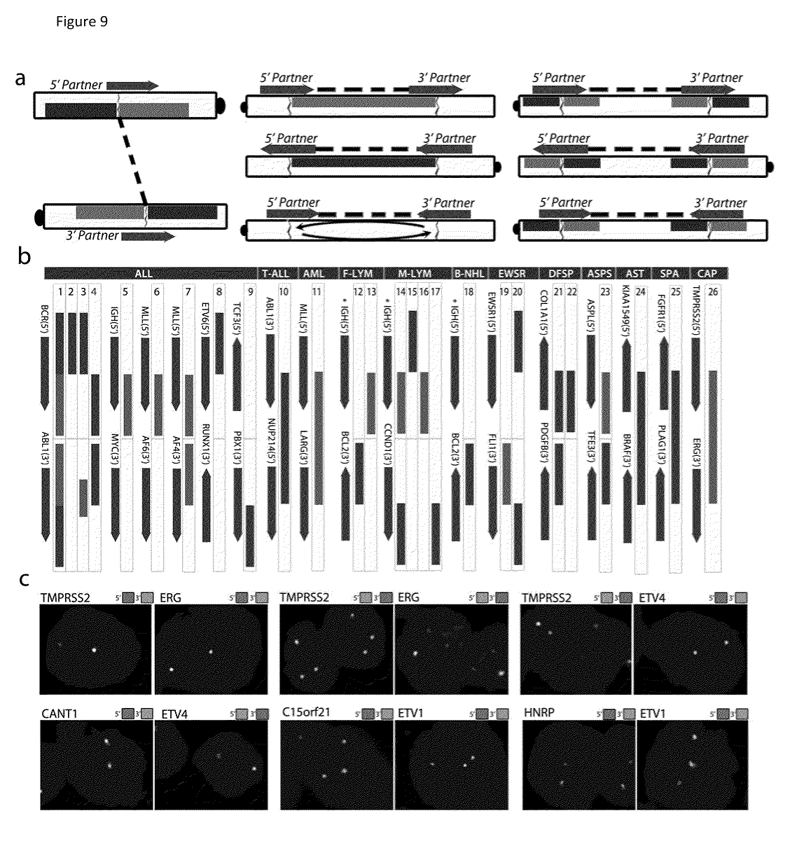

High-throughput copy number data was next used to explore the genomic imbalance pattern that could inform unidentified gene fusions. Using leukemia as a genetic model, the recurrent fusion genes were studied in a high-resolution single nucleotide polymorphism microarray (SNP array) dataset with 304 leukemia samples (Fuhrer et al., Biochem. Biophys. Res. Commun. 224:289 1996; Kharas, J. din. Invest. 118:3039 2008). A total of 157 samples were annotated with seven gene fusions in this dataset (Table 6). The percentage of unbalanced fusions ranged from 21.2% to 94.1% for different fusions, with most TCF3-PBX1 fusions identifiable by unbalanced breakpoints (FIG. 3a). The physical lengths of amplifications/deletions associated with fusion genes ranged between 0.08-84.21 Mb (averaging 19.7 Mb). Heterogeneity in the genomic aberrations generating gene fusions was observed. Often two fusion partners were found to possess different degrees of copy number gain or loss; elsewhere one fusion partner harbors a balanced translocation while the other partner has an unbalanced translocation. An association analysis of unbalanced breakpoints with fusion gene placements revealed a consistent genetic pattern—copy number increases generally affect the 5′ region of 5′ partners and the 3′ region of 3′ partners, whereas deletions generally remove the 3′ region of 5′ fusion partners and the 5′ region of 3′ partners. Of 56 samples with 7 unbalanced fusions in this dataset, 55 samples follow this pattern (FIG. 3b, Table 7). The data for 36 leukemia cell lines (Fuhrer et al, supra) and associated gene fusions from published sources (Mullighan, Nature 453:110 2008); was analyzed and 11 of 12 unbalanced fusions from these cell lines were found to follow this pattern (FIG. 3c, Table 8). This pattern was called the “fusion breakpoint principle”. Based on this reasoning, one can deduce an inferred principle for the unbalanced gene fusions within the same chromosome (FIG. 9a middle, right panels): If the 5′ partner locates at the 5′ side of the 3′ partner within the same DNA strand, the fusion can not be delineated by copy number increase; if the 5′ partner locates at the 3′ side of the 3′ partner within the same strand, the fusion can not be generated by deletions. If the 5′ and 3′ partners locate in different strands (inversion), the fusion can not be generated by simple interstitial deletions or copy number increase.

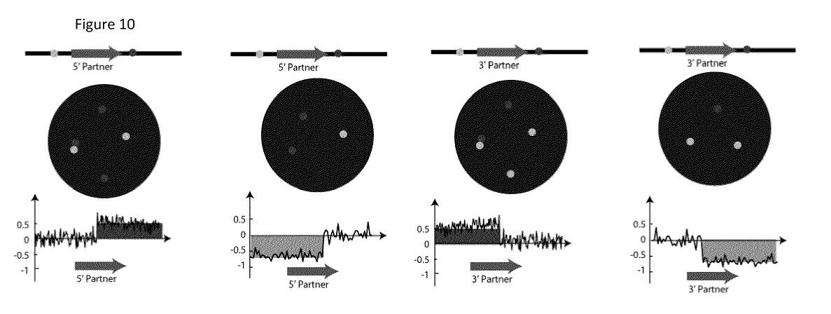

While the fusion breakpoint principle can be inferred based on conventional cytogenetics analysis, unlike G-banding and fluorescence in situ hybridization (FISH), array-based high-throughput genomic data loses balanced genomic translocation information, and may misrepresent individual cases of complex genomic rearrangements (See FIG. 10).

To confirm the breakpoint principle, a large-scale meta-analysis of recurrent gene fusions was performed based on high-resolution array CGH/SNP datasets annotated with gene fusions, as well as literature curation (Table 2). In total, 276 tumor samples were identified as having unbalanced fusions, including 85 leukemia, 15 lymphoma, 23 sarcoma, and 153 epithelial tumor samples. Although diverse breakpoint patterns were observed on these samples (FIG. 9b), the unbalanced fusions from 273 samples conformed to the principle (98.9%). Furthermore, the inferred principle was confirmed by analyzing the reports for all unbalanced intra-chromosome fusions from the Mitelman database (Mullighan, Nautre 446:758 2007) (See Table 10).

### Application of ConSig Technology to New Fusion Discovery

To demonstrate the application of those principles to new fusion discovery, NGS data and the large-scale genomic data from lung cancer was analyzed. First the ConSig Score was used to nominate biologically important fusion candidates from paired-end transcriptome data from lung cancer cell lines run in a single lane on an Illumina Genome Analyzer II flowcell. The chimeric paired reads were extracted from the paired-end libraries, and the the 3′ partners were ranked by rConSig score. This was first tested on the H2228 cell line known to harbor the recurrent EML4-ALK fusion. Rating 3′ partners of paired-end chimeras by rConSig score revealed EML4-ALK as the top on the H2228 cell line, which was supported by six mate pairs (FIG. 4a, left). This showed the effectiveness of the rConSig score in preferentially nominating driver gene fusions from numerous paired-end chimeras.

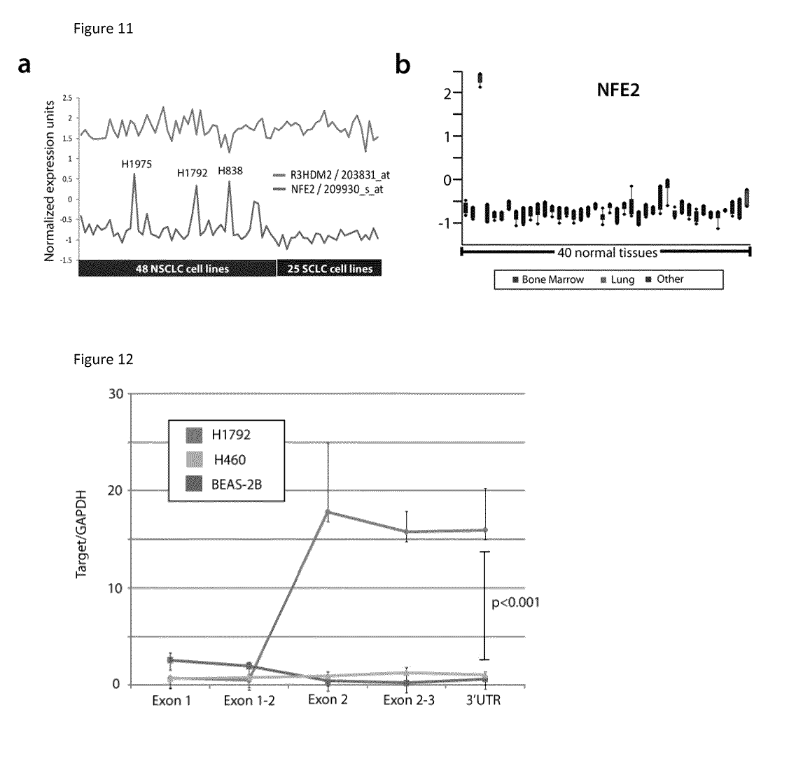

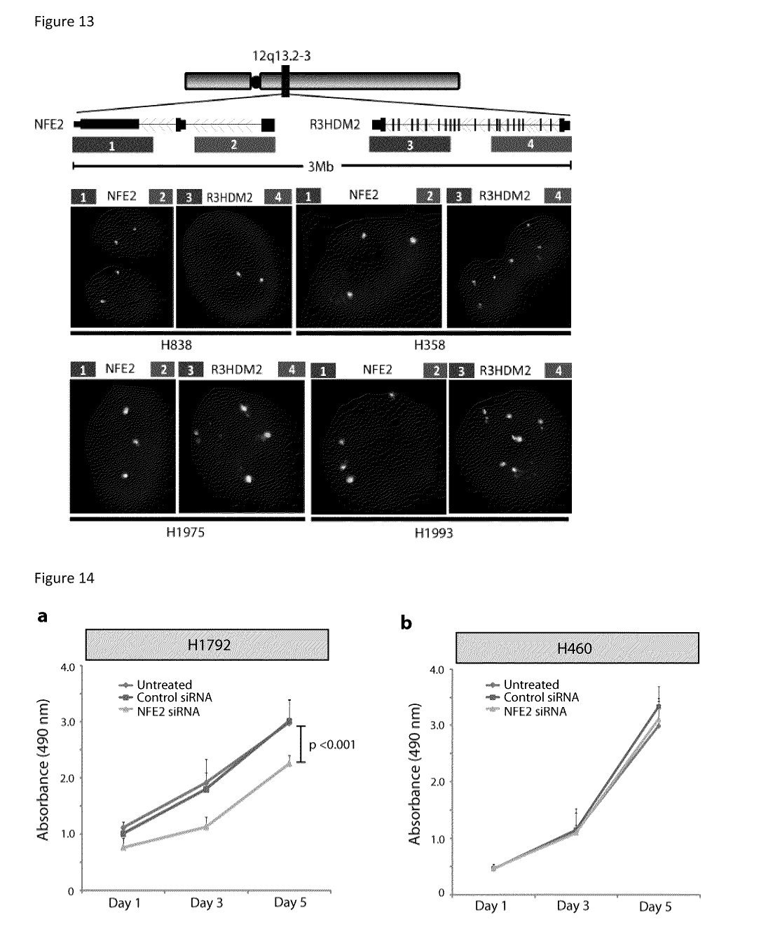

This method was then used to reveal biologically meaningful chimeras from the transcriptome data of 12 lung cancer cell lines. While there were 530 gene fusions in total supported by more than two paired reads, the 3′ rConSig Score prioritized R3HDM2-NFE2 as the lead in the H1792 lung cancer cell line (supported by three paired reads, FIG. 4a, right), and this fusion was confirmed by quantitative RT-PCR (qRT-PCR) (FIG. 4c), conventional capillary sequencing and interphase FISH, the latter of which showed high copy-number gain of R3HDM2-NFE2 in H1792 (FIG. 4e). Consistent with previous microarray data on lung cancer cell lines (FIG. 11a), qRT-PCR also revealed marked over-expression of NFE2 on H1792 and several additional lung adenocarcinoma cell lines (FIG. 4c); however, no rearrangements were detected in these samples by FISH, indicating other mechanisms activating NFE2 expression (FIG. 13).

The R3HDM2-NFE2 fusion was predicted to encode the full-length open reading frame of NFE2, with only untranslated promoter sequences contributed from R3HDM2 (FIG. 4b), and exon-walking qRT-PCR demonstrated the specific over-expression of the NFE2 coding exons 2-3 under the regulation of the R3HDM2 promoter (FIG. 12). In H1792, knock-down of NFE2, which encodes a transcription factor normally expressed during erythropoiesis, resulted in a marked decrease in cell proliferation and to a lesser extent cell invasion (FIG. 4g), whereas no effect was seen in H460, which has low levels of endogenous NFE2 (FIG. 14). Furthermore, analysis of SNP array data for 139 lung adenocarcinoma tissues revealed copy number gain at the 3′ NFE2 locus in 2 lung cancer patients (FIG. 4d), indicating recurrent aberrations involving the NFE2 locus in this cancer. FISH analysis was performed on a lung cancer tissue microarray (TMA) comprised of a different cohort of 76 lung adenocarcinoma samples, which confirmed recurrent NFE2 rearrangements in two patients (FIG. 4f).

TABLE 1
The compendia of molecular concepts for integrative
functional analysis of fusion genes. Four classes of
molecular concepts were compiled from 6 sources.
Connectivity represents the total number of concept to
gene connections in each concept type.

| | | | Concepts | Connectivity |
|---|---|---|---|---|
| Class | Source | Type | (n) | (n) |
| | | Biologic process | 3920 | 46530 |
| Annotation | Gene | Cellular component | 732 | 42463 |
| | Ontology | | | |
| | | Molecular function | 2561 | 47026 |
| | Biocarta | Signaling pathways | 263 | 4459 |
| | KEGG | Metabolic pathways | 112 | 2985 |
| Pathways | | Signaling pathways | 2456 | 52238 |
| | Reactome | Biochemical | 5450 | 44347 |
| | | reactions | | |
| Interactions | HPRD | Protein interaction | 7819 | 37206 |
| | | sets | | |
| Domains | Entrez | Conserved domains | 5650 | 5693 |
| | Gene | | | |

TABLE 2
Meta-analysis of unbalanced gene fusions in multiple human cancers to test the fusion
breakpoint principle.

| | | | | Total Samples | | Follow the | |
|---|---|---|---|---|---|---|---|
| Cancer type | First Author | Citation | Platform | with fusions | Unbalanced | principle | |
| ALL CML | Mulighan CG | Nature 2007; 445:758 | Affymatrix 500K aSNP | 185 | 68 | 66 | |
| ALL | Paulsson K | PNAS 2008; 105:6708 | Affymatrix 500K aSNP | 13 | 6 | 5 | |
| ALL | Kuiper KP | Leukemia 2007; 21:1258 | Affymatrix 100K aSNP | 6 | 4 | 4 | |
| T-ALL | Graux C | Nat Gemet 2004; 36:1084 | CGH | 6 | 6 | 6 | |
| AML | Kourlas PJ | PNAS 2000; 97:2145 | CGH | 1 | 1 | 1 | |
| NHL | Ferrerra BI | Haematologica 2008; 93:670 | Agilent 44B aCGH | 48 | 8 | 8 | |
| B-NHL | Galteland E | Leukemia 2005; 19:2313 | BAC aCGH | 18 | 7 | 7 | |
| EWS | Ferrerra BI | Oncogene 2008; 27:2084 | Agilent 44B aCGH | 25 | 2 | 2 | |
| DFSP | Linn SC | Am J Pathol 2003; 163:2383 | Standford aCGH | 7 | 7 | 7 | |
| DFSP | Kaw S | Cytogener Genome Res 2006 | Agilent 13l aCGH | 7 | 5 | 5 | |
| ASPS | Ladanyi M | Oncogene 2001; 20:48 | FISH | 12 | 9 | 9 | |
| AST | Jones DT | Cancer Res 2008; 68:8673 | MHP 1Mb aCGH | 29 | 29 | 29 | |
| SPA | Persson F | Oncogene 2008; 27:3072 | Agilent 44B aCGH | 11 | 10 | 10 | |
| CaP | Liu W | Gene Chrom Canc 2007; 46:972 | Affymetrix 500K aSNP | 41 | 16 | 16 | |
| CaP | Permer S | Cancer Res 2006; 66:8337 | FISH | 65 | 38 | 38 | |
| CaP | Wang XS | This study | FISH | 104 | 60 | 60 | |
| | | | TOTAL: | 578 | 276 | 273 | (98.9%) |

Abbreviations:
ALL, acute lymphoblastic leukemia;
CML, chronic mylogenous leukemia;
AML, acute mylogenous leukemia;
NHL, non-Hodgkin's lymphoma;
EWS, Ewing's sarcoma;
DFSP, dermatofibrosarcoma protruberans;
ASPS, Alveolar soft part sarcoma;
AST, astrocytoma;
SPA, Salivary Pleomorphic Adenoma;
CaP, prostate cancer.

### The Functional Significance of Fusion-Interaction Hubs and Their Potential as Drug Targets

To explore the functional role of most significant shared interacting genes with the connecting fusion genes, the fusion-interaction network was resolved by setting the p value to 10⁻⁷. After spring-embedded relaxing using the VisANT program (Hu et al. Nucleic Acids Res 35, W625-632 (2007)), the FI network was spread into six major clusters, which join gene fusions from similar tumor entities (FIG. 1d). The shared interacting genes with the greatest statistical significance in each subset of connected fusions were designated as fusion-interaction hubs in each cluster. In cluster i, this approach identified GATA3 as the hub for a subset of T cell receptor fusion partners—LYL1, TAL1, TAL2, LMO1 and LMO2, which are normally transcriptional cofactors for GATA3 (Ono et al., Mol Cell Biol 18, 6939-6950 (1998)). Whereas MEIS1 was the center of another subset of NUP98 fusion partners, the HOX family genes, which were reported to collaborate with MEIS1 in AML transformation (Thorsteinsdottir et al., Mol Cell Biol 21, 224-234 (2001)).

In clusters ii and iii, CDK6 and CTNNB1 join two distinct subsets of immunoglobulin fusion partners, exemplified by cyclins and BCL genes. CDK6 is known to form a complex with cyclins, that phosphorylates and inhibits Rb; whereas CTNNB1 is a known upstream protein of the BCL genes (Sampietro, J. et al. Mol Cell 24, 293-300 (2006)). In cluster v, PIK3R1 connects most of BCR and ETV6 fusion partners. This indicated that the central role of PIK3R1 in mediating the signaling of these fusion proteins. HDAC1, the hub for a subset of RUNX1 fusions in cluster iv, is normally a co-repressor of the RUNX1 partners—RUNX1T1, CBFA2T3, MDS1 and EVI1 (Senyuk, V. et al. Oncogene 21, 3232-3240 (2002)). Moreover, ERG and RPS6KA5 (MSK1) are hubs of ESWR1 fusions in cluster vi; the latter was reported to regulate EWSK1 partners—CREB1, ETV1 and ATF1 (Janknecht, Oncogene 22, 746-755 (2003)). The consistent functional relationship between a FI hub and a fusion partner family indicates this hub is a functional factor joining this family. This implies the role of FI hubs in mediating the function of the fusion proteins, and the benefits of mining for drug targets from shared interacting hubs to block multiple fusions. For example, HDAC1 is recruited by 3′ partners of RUNX1 resulting in dominant-negative effect over wild-type RUNX1 (Maki et al. Cancer Sci 99, 1878-1883 (2008); Hart et al., Haematologica 87, 1307-1323 (2002), and thus provides a target to block RUNX1 fusions (FIG. 1d).

The significance of the FI hubs in molecular targeting was verified by the therapeutic effect of their ablating drugs on the specific tumor entities where the connected gene fusions occur. The literature was investigated for the hubs that have blocking reagents, PIK3R1, CDK6, and HDAC1, according to the drug target database (Wishart, D. S. et al. Nucleic Acids Res 34, D668-672 (2006).). A recent report revealed that PIK3R1 and PIK3R2 are the specific PI3K isoforms that mediate transformation of BCR-ABL1 positive pre-B-ALL, and PI3K ablation by PI-103 can block BCR-ABL leukemogenesis in mice (Kharas, M. G. et al. J Clin Invest 118, 3038-3050 (2008)). Moreover, the HDAC1-3 inhibitor, Vorinostat, was reported to be effective in AML patients by a recent study (Garcia-Manero, G. et al. Blood 111, 1060-1066 (2008)), whereas Flavopiridol, an inhibitor of CDKs, was under clinical trial for the treatment of relapsed or refractory B cell lymphoma (Dunleavy, NCI—Center for Cancer Research: NCI-07-C-0081 (2007-2009)).

### The Application of ConSig Technology to Deep Sequencing Data Analysis

The ConSig technology preferentially identifies biologically important genes in cancer. This is particularly useful in the analysis of a large number of putative chimeras generated by next generation sequencing data to filter secondary fusions. This is especially useful for evaluating the genes involved in both fusion and point mutations (mixed type cancer genes), for example, a fusion involving EGFR gene is considered as biologically important because of the prior knowledge of EGFR point mutation in cancer.

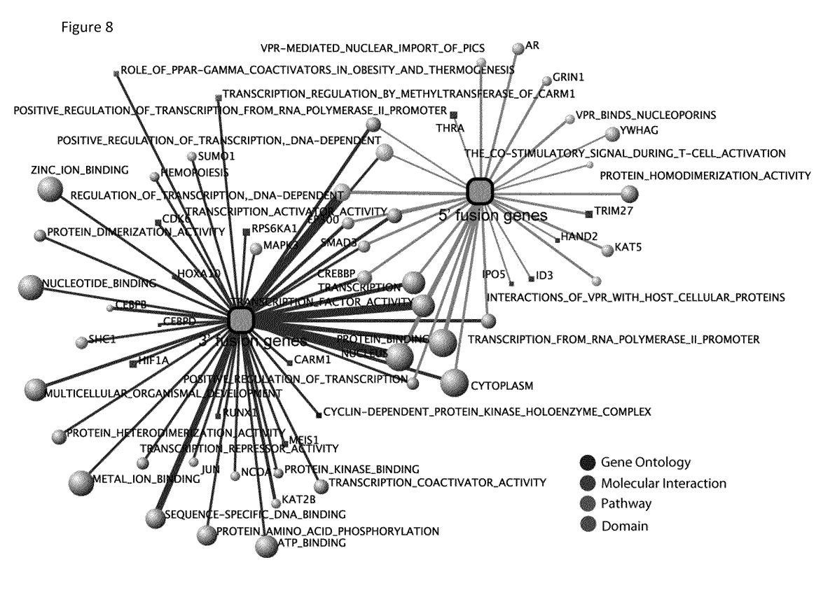

Moreover, the 3′ fusion partners display more distinctive signature concepts than the 5′ partners (FIG. 8), therefore the ConSig technology is more discriminative in evaluating 3′ genes. In practice, the 3′ partners of fusion chimeras are first rated by rConSig scores, and then the 5′ partners are rated by rConSig score to supplement this analysis.

### Confirmation of the Fusion Breakpoint Principle

While the fusion breakpoint principle can be inferred based on conventional cytogenetics analysis, the net output of high-throughput genomic measurement was different from G-banding and FISH, where the balanced genomic relocation information was lost. FIG. 10 demonstrates the possible complex chromosome rearrangements generating contradictory cases to the breakpoint principle on array CGH, but not on FISH data. For example, the THP-1 cell line harbors a MLL-AF9 fusion with duplication of the 3′ MLL gene that deviates from the principle. Studying public spectral karyotyping data revealed complex translocations between chr9 and chr10 resulting in possible three-way fusions involving the MLL gene (Odero et al., Genes Chromosomes Cancer 29, 333-338 (2000)).

A large-scale meta-analysis of unbalanced gene fusions based on high-resolution array CGH/SNP datasets annotated with gene fusions, as well as literature curation (Table 2, FIG. 9b) was performed. Analyses of four independent leukemia datasets and two lymphoma datasets was performed. Of 32 samples with 10 different unbalanced fusions in these datasets, 31 follow the principle. Analyses of the known fusions in mesenchymal and epithelial tumors also yielded strong supporting evidences. In four sarcoma datasets, three unbalanced fusions were identified in 23 samples, including EWSR1-FLI1 in Ewing's sarcoma, COL1A1-PDGFB fusion in dermatofibrosarcoma protuberans (DFSP), and ASPSCR1-TFE3 in alveolar soft part sarcoma (ASPS). None of these contradict the principle. In salivary adenoma, a FGFR1-PLAG1 fusion is found with interstitial duplications (Persson, F. et al. Oncogene (2007), whereas TMPRSS2-ERG fusion in prostate cancer is frequently reported having heterozygous deletions (Perner, S. et al. Cancer Res 66, 8337-8341 (2006); Liu, W. et al. Genes Chromosomes Cancer 46, 972-980 (2007)). Both are intra-chromosome gene fusions, but the genomic placements of genes in the two fusions are clearly opposite. This clearly demonstrated the inferred principle. The same pattern was also observed in the recurrent KIAA1549-BRAF fusion in astrocytoma (AST) (Jones, D. T. et al. Cancer Res 68, 8673-8677 (2008)). Furthermore, the reports were analyzed for all unbalanced intra-chromosome fusions from the Mitelman database, and it was confirmed that the inferred principle is the adherent genetic factor that determines the nature of genomic imbalances associated with these fusions (Table 10).

To further test this principle on prostate cancer, where unbalanced fusions are less studied by conventional cytogenetics, all fluorescence in situ hybridization performed on prostate cancer for ETS family gene fusions with break-apart probes was reviewed. A total of 60 samples with 5 unbalanced ETS fusions were found from 238 prostate cancer samples, including TMPRSS2-ERG, TMPRSS2-ETV4, C15orf21-ETV1, HNRP-ETV1, and CANT1-ETV4; no contradictory case was identified (FIG. 9c, and Table 11).

TABLE 3
Genbank sequences indicating distinct domain patterns of known gene fusions. Each
domain pattern was defined as the unique domain architecture generated by the fusion of two wild-
type proteins; fusion of the same gene partners could generate different domain patterns due to the
difference of fusion breakpoints.

| Fusion | Pattern No. | Genbank Accessions |
|---|---|---|
| AFF1-MLL | 1 | AF487906; AF492831; |
| AFF1-MLL | 2 | AF177238; AF177239; |
| AKAP9-BRAF | 1 | AY803272; |
| ASPSCR1-TFE3 | 1 | AY034077; |
| BCR-ABL1 | 1 | AM491362(e6a2); |
| BCR-ABL1 | 2 | EU236680(e14a3); S72478(e14a3); |
| BCR-ABL1 | 3 | EU216071(e14a2, YS); M25946(e14a2, K562, CML); M30829(e14a2, K562); |
| | | M30832(e14a2, EM2, CML); |
| BCR-ABL1 | 4 | AF487522(e18a2, CML); |
| BCR-ABL1 | 5 | AM491359(e13a3); AY043457(e13a3, CML); |
| BCR-ABL1 | 6 | AI131467(e13a2); EF158045(e13a2, SCA and CML); EU216066(e13a2, |
| | | CML); |
| BCR-ABL1 | 7 | AM491360(e14a3); |
| BCR-ABL1 | 8 | AI131466(e14a2); M13096(e14a2, K562); |
| BCR-ABL1 | 9 | AM491363(e19a2); |
| BCR-ABL1 | 10 | AM491361(e1a3); S72479(e1a3, ALL); |
| BCR-ABL1 | 11 | AF113911(e1a2); M17541(e1a2, ALL); M19730(e1a2, ALL); |
| | | X06418(e1a2, ALL); X07537(e1a2, ALL); |
| BIRC3-MALT1 | 1 | AF123094; |
| BRD4-C15orf55 | 1 | AY166680; |
| CBFB-MYHI1 | 1 | AF249897; AF249898; |
| CCDC6-RET | 1 | D90075; |
| CD74-ROS1 | 1 | EU236945; |
| CDK6-MLL | 1 | AF492830; |
| CHCHD7-PLAG1 | 1 | DQ478931; DQ478932; |
| CNBP-USP6 | 1 | AY624556; |
| COL1A1-PDGFB | 1 | X98709; X98710; Y15913; Y15917; Y15918; Y15919; Y15921; |
| COL1A1-PDGFB | 2 | X98707; X98708; Y08643; Y15914; Y15915; Y15916; Y15920; Y16346; |
| DAZAP1-MEF2D | 1 | AY678451; |
| DDX10-NUP98 | 1 | AB001342; |
| DDX10-NUP98 | 2 | AB001343; |
| ELL-MLL | 1 | DQ437655; |
| EML4-ALK | 1 | AB274722; |
| EML4-ALK | 2 | AB275889; |
| ETV6-ABL1 | 1 | Z35761; |
| ETV6-MN1 | 1 | X85024; X85026; |
| ETV6-NTRK3 | 1 | AF041811; AF125808; |
| EWSR1-DDIT3 | 1 | X92120; |
| EWSR1-ERG | 1 | S72621; S72622; S72865; |
| EWSR1-ETV4 | 1 | U35622; |
| EWSR1-FLI1 | 1 | AF327066; S62665; S72620; |
| EWSR1-NR4A3 | 1 | AF524261; S81242; |
| EWSR1-WT1 | 1 | S74529; |
| EWSR1-WT1 | 2 | S79672; |
| FGFR1-BCR | 1 | AJ298917; |
| FGFR1-PLAG1 | 1 | EF525168; EF525169; |
| FUS-ATF1 | 1 | AJ295163; |
| FUS-DDIT3 | 1 | AJ301611; |
| FUS-DDIT3 | 2 | AJ301612; S62138; S75762; S75763; X71427; |
| FUS-ERG | 1 | S77574; |
| GOLGA5-RET | 1 | X15786; |
| HMGA2-RAD51L1 | 1 | AY138857; AY138858; AY138859; |
| HNRNPA2B1-ETV1 | 1 | EF632110; |
| HOOK3-RET | 1 | DQ104207; |
| MAML2-CRTC1 | 1 | AY186998; |
| MAPRE1-MLL | 1 | AY752859; |
| MEF2D-DAZAP1 | 1 | AY675556; |
| MKL1-RBM15 | 1 | AF364036; |
| MLL-AFF1 | 1 | AF024541; AF177236; AF177237; DQ451148; |
| MLL-AFF1 | 2 | AF031404; AF487905; AF492832; S67825; |
| MLL-AFF3 | 1 | AF422798; |
| MLL-CBL | 1 | AY125965; |
| MLL-EPS15 | 1 | AF331760; |
| MLL-EPS15 | 2 | AY187922; |
| MLL-GAS7 | 1 | AF231998; AF231999; |
| MLL-GAS7 | 2 | AF231995; AF231996; AF231997; |
| MLL-GMPS | 1 | AF297746; AF297748; |
| MLL-GMPS | 2 | AF297747; AF297749; |
| MLL-KIAA0284 | 1 | AM422012; |
| MLL-MAML2 | 1 | AJ972402; DQ084494; DQ886023; |
| MLL-MAML2 | 2 | DQ886024; |
| MLL-MAPRE1 | 1 | AY752858; |
| MLL-MLLT1 | 1 | DQ224341; |
| MLL-MLLT1 | 2 | AF331759; AY040555; |
| MLL-MLLT1 | 3 | DQ224342; |
| MLL-MLLT1 | 4 | AY187921; |
| MLL-MLLT10 | 1 | AF272375; AF272383; |
| MLL-MLLT10 | 2 | AY187923; |
| MLL-MLLT10 | 3 | AF272376; AF272384; AF272385; |
| MLL-MLLT3 | 1 | EF406122; |
| MLL-MLLT3 | 2 | S82034; |
| MLL-MLLT4 | 1 | DQ387206; |
| MLL-MLLT6 | 1 | S72604; |
| MLL-PICALM | 1 | AF477006; |
| MLL-SEPT5 | 1 | AF061154; |
| MLL-SEPT6 | 1 | AF450279; AF512943; AF512944; AF512945; AF512946; |
| MLLT10-PICALM | 1 | AF060927; AF060930; AF060931; |
| MLLT1-MLL | 1 | AF373587; |
| MN1-ETV6 | 1 | X85025; X85027; |
| MYST3-ASXZ2 | 1 | AB084281; |
| MYST3-CREBBP | 1 | AJ251843; |
| MYST3-NCOA2 | 1 | EF374064; |
| MYST4-CREBBP | 1 | AJ299261; |
| NIN-PDGFRB | 1 | AY764156; |
| NOL1-TCF3 | 1 | EU155120; |
| NTRK3-ETV6 | 1 | AF125809; |
| NUP98-DDX10 | 1 | AB000267; |
| NUP98-DDX10 | 2 | AB000268; |
| NUP98-HOXC13 | 1 | AJ438986; |
| NUP98-HOXD13 | 1 | AB038155; |
| NUP98-PRRX2 | 1 | AY662674; |
| NUP98-RAP1GDS1 | 1 | AF133331; AF133332; |
| PAX3-FOXO1 | 1 | AF178854; BC008826; U02308; U02368; |
| PAX3-NCOA1 | 1 | AY633656; |
| PAX5-ETV6 | 1 | DQ841178; |
| PAX5-FOXP1 | 1 | DQ845346; |
| PAX5-ZNF521 | 1 | DQ845345; |
| PCM1-RET | 1 | AJ297349; |
| PICALM-MLLT10 | 1 | EF051633; |
| PML-RARA | 1 | M73779; S50916; |
| PRKAR1A-RET | 1 | L03357; |
| RARA-PML | 1 | M82827; |
| RBM15-MKL1 | 1 | AJ303089; |
| RPN1-EVI1 | 1 | AF310158; |
| RUNX1-MDS1 | 1 | S69002; |
| RUNX1-RUNX1T1 | 1 | AX813476; AX813478; D13979; D14822; D14823; S78158; S78159; |
| RUNX1-SH3D19 | 1 | EU093086; EU093087; |
| SLC34A2-ROS1 | 1 | EU236946; EU236947; |
| SLC45A3-ETV1 | 1 | EF632109; |
| SLC45A3-ETV5 | 1 | EU314932; |
| SS18-SSX1 | 1 | S79325; |
| SS18-SSX2 | 1 | X79200; |
| SS18-SSX4 | 1 | AF114234; |
| TAF15-NR4A3 | 1 | AF162670; AJ243810; AJ245932; |
| TCF12-NR4A3 | 1 | AF289510; |
| TCF3-PBX1 | 1 | AY311345; M31522; |
| TFG-ALK | 1 | AF125093; AF143407; AF390893; |
| TFG-NR4A3 | 1 | AY532911; |
| TFG-NTRK1 | 1 | X85960; |
| TMPRSS2-ERG | 1 | DQ204773; DQ831522; EU090248; |
| TMPRSS2-ETV1 | 1 | DQ204770; |
| TMPRSS2-ETV5 | 1 | EU314929; EU314930; EU314931; |

TABLE 4
Top hub genes shared by 3′fusion partner families as revealed by
significant overlapping statistics. “x, k, N” correspond to the
variables demonstrated in the algorithm of hypergeometric
statistics (FIG. 1a)

| | | | | Total | |
|---|---|---|---|---|---|
| | | | 3′partners | interacting | |
| | | | binding | genes of | |
| | | Hub | the | the hub | |
| 5′Partner | 3′partners(x) | Genes | hubs (k) | gene (N) | P value |
| NUP98 | 20 | MEIS1 | 6 | 21 | 3.77E−16 |
| IGL@ | 11 | CDK6 | 4 | 20 | 1.52E−11 |
| MYST3 | 3 | CARM1 | 3 | 11 | 1.57E−11 |
| IGH@ | 36 | RUNX1 | 5 | 25 | 2.36E−11 |
| IGL@ | 11 | RUNX1 | 4 | 25 | 3.96E−11 |
| IGL@ | 11 | DMTF1 | 3 | 4 | 6.26E−11 |
| BCR | 4 | PIK3R1 | 4 | 126 | 9.54E−11 |
| TRD@ | 8 | GATA3 | 3 | 7 | 1.86E−10 |
| MYST3 | 3 | HIF1A | 3 | 29 | 3.47E−10 |
| TRB@ | 15 | LMO1 | 3 | 6 | 8.63E−10 |
| EWSR1 | 13 | ERG | 3 | 7 | 9.49E−10 |
| IGL@ | 11 | CDKN1A | 4 | 55 | 1.06E−09 |
| MYST3 | 3 | NCOA2 | 3 | 44 | 1.26E−09 |
| IGH@ | 36 | CTNNB1 | 6 | 122 | 1.30E−09 |
| IGL@ | 11 | AKAP8 | 3 | 9 | 1.31E−09 |
| TRB@ | 15 | GATA3 | 3 | 7 | 1.51E−09 |

TABLE 5
PCR primers used in this study.

| | Accession | Refseq | Type | Sequence |
|---|---|---|---|---|
| Primer | Number | (ucsc) | | (5′→3′) |
| R3HDM2-NFE2 fusion | NM_014925- | uc001 snt- | Forward | ACTCATGGAGGCTGAGCATT |
| (exon 2 to exon 2) | NM_006163 | uc001 sfq | | |
| R3HDM2-NFE2 fusion | NM_014925- | uc001 snt- | Reverse | AGCTCGGTGATGGACATGAT |
| (exon 2 to exon 2) | NM_006163 | uc001 sfq | | |
| NFE2 (exon 1) | NM_006163 | uc001 sfq | Forward | AGCAGGGTGACCCCTGATGTTGCCC |
| NFE2 (exon 1) | NM_006163 | uc001 sfq | Reverse | ACTCCCCCAAACTGTTTTCCTGGCT |
| NFE2 (exon 1 − exon 2) | NM_006163 | uc001 sfq | Forward | AGCAGGGTGACCCCTGATGTTGCCC |
| NFE2 (exon 1 − exon 2) | NM_006163 | uc001 sfq | Reverse | TGGTCCAGGTTCCCGGAAAGCCCA |
| NFE2 (exon 2) | NM_006163 | uc001 sfq | Forward | TGGCCCAGTAGGATGTCCCCGTGT |
| NFE2 (exon 2) | NM_006163 | uc001 sfq | Reverse | GTGGACAGCTGTATCACCCTGTTCCT |
| NFE2 (exon 2 − exon 3) | NM_006163 | uc001 sfq | Forward | TCCCCAGCAGAGCAGGAACAGGGTGA |
| NFE2 (exon 2 − exon 3) | NM_006163 | uc001 sfq | Reverse | AAGGTATGGAGCTGGGGCTTGGGGCT |
| NFE2 (3′UTR) | NM_006163 | uc001 sfq | Forward | CTGAATCTCTTGAGCTGGAGG |
| NFE2 (3′UTR) | NM_006163 | uc001 sfq | Reverse | GCTGGCAAGGTATAGTTGGAGT |
| GAPDH | NM_002046 | — | Forward | TGCACCACCAACTGCTTAGC |
| GAPDH | NM_002046 | — | Reverse | GGCATGGACTGTGGTCATGAG |

TABLE 6
The summary of gene fusions reported in the leukemia array SNP dataset (GSE9113)

| | 5′ Partner | | 3′ Partner | Total Fusion | Unbalanced | Unbalanced/ |
|---|---|---|---|---|---|---|
| 5′ Partner | Cytoband | 3′ Partner | Cytoband | Samples (n) | Fusions (n) | Total % |
| BCR | 22q11.23 | ABL1 | 9q34.12 | 43 ALL | 9 ALL | 21.2 |
| | | | | 23 CML | 5 CML | |
| EIV6 | 12p13.2 | RUNXI | 21q22.12 | 48 | 17 | 35.4 |
| MLL | 11q23.3 | Multiple | | 22 | 5 | 22.7 |
| TCP3 | 19p13.3 | PBXI | 1q23.3 | 17 | 16 | 94.1 |
| PAX3 | 9p13.2 | ETV6 | 12p13.2 | 2 | 2 | 100 |
| PAX3 | 9p13.2 | FOXP1 | 3p14.1 | 1 | 1 | 100 |
| PAX3 | 9p13.2 | ZNF521 | 18q11.2 | 1 | 1 | 100 |

TABLE 7
Analysis of the genomic imbalances associated with each gene fusion identified from
the leukemia array SNP dataset (GSE9113).

| | | | Copy number abberation at | Copy number abberation at 3′ |
|---|---|---|---|---|
| GEO accession | SAMPLE_ID | FUSION | 5′ gene locus | gene locus |
| GSM235572 | #1 | BCR-ABL | N | N |
| GSM235734 | #10 | BCR-ABL | N | N |
| GSM235735 | #11 | BCR-ABL | 5′amp->T; 3′del->Rgr | 5′del->PPP2R4; 3′amp->T |
| GSM235736 | #12 | BCR-ABL | N | N |
| GSM235738 | #13 | BCR-ABL | N | N |
| GSM235740 | #14 | BCR-ABL | 5′amp->T | 3′amp->T |
| GSM235743 | #15 | BCR-ABL | N | N |
| GSM235747 | #16 | BCR-ABL | 3′del->LOC649264 | N |
| GSM235751 | #17 | BCR-ABL | N | N |
| GSM235753 | #18 | BCR-ABL | N | N |
| GSM235801 | #19 | BCR-ABL | N | N |
| GSM235617 | #2 | BCR-ABL | 5′amp->T | 3′amp->T |
| GSM235809 | #20 | BCR-ABL | N | N |
| GSM235865 | #21 | BCR-ABL | N | N |
| GSM235810 | #22 | BCR-ABL | N | N |
| GSM235713 | #23 | BCR-ABL | N | N |
| GSM235714 | #24 | BCR-ABL | N | N |
| GSM235715 | #25 | BCR-ABL | N | N |
| GSM235716 | #26 | BCR-ABL | N | N |
| GSM235717 | #27 | BCR-ABL | N | N |
| GSM235718 | #28 | BCR-ABL | N | N |
| GSM235719 | #29 | BCR-ABL | N | N |
| GSM235645 | #3 | BCR-ABL | N | N |
| GSM235720 | #30 | BCR-ABL | N | N |
| GSM235721 | #31 | BCR-ABL | N | N |
| GSM235722 | #32 | BCR-ABL | N | N |
| GSM235723 | #33 | BCR-ABL | N | N |
| GSM235724 | #34 | BCR-ABL | 5′amp->MAPK1 | 3′amp->Chr9: 133236046 |
| GSM235725 | #35 | BCR-ABL | N | N |
| GSM235726 | #36 | BCR-ABL | N | N |
| GSM235727 | #37 | BCR-ABL | N | N |
| GSM235728 | #38 | BCR-ABL | N | N |
| GSM235729 | #39 | BCR-ABL | N | N |
| GSM235664 | #4 | BCR-ABL | N | N |
| GSM235730 | #40 | BCR-ABL | 5′amp->T | 3′amp->T |
| GSM235731 | #41 | BCR-ABL | 3′del->UPB1 | 5′del->ZER1 |
| GSM235732 | #42 | BCR-ABL | N | N |
| GSM235733 | #43 | BCR-ABL | N | N |
| GSM235680 | #5 | BCR-ABL | N | 5′del->5′region |
| GSM235693 | #6 | BCR-ABL | 3′del->T | N |
| GSM235702 | #7 | BCR-ABL | N | N |
| GSM235767 | #8 | BCR-ABL | 5′amp->T | 3′amp->T |
| GSM235812 | #9 | BCR-ABL | N | N |
| GSM236531 | #10-AP | CML(BCR-ABL1) | N | N |
| GSM236532 | #10-CP | CML(BCR-ABL1) | N | N |
| GSM236534 | #11-AP | CML(BCR-ABL1) | N | N |
| GSM236533 | #11-CP | CML(BCR-ABL1) | N | N |
| GSM236536 | #12-AP | CML(BCR-ABL1) | N | N |
| GSM236535 | #12-CP | CML(BCR-ABL1) | N | N |
| GSM236537 | #12-CP2 | CML(BCR-ABL1) | N | N |
| GSM236538 | #13-CP | CML(BCR-ABL1) | N | N |
| GSM236539 | #13-CP2 | CML(BCR-ABL1) | N | N |
| GSM236540 | #14-BC | CML(BCR-ABL1) | N | N |
| GSM236541 | #14-Rem | CML(BCR-ABL1) | N | N |
| GSM236544 | #15-BC | CML(BCR-ABL1) | N | N |
| GSM236542 | #15-CP | CML(BCR-ABL1) | N | N |
| GSM236543 | #15-CP2 | CML(BCR-ABL1) | N | N |
| GSM236547 | #16-BC | CML(BCR-ABL1) | N | N |
| GSM236548 | #16-BC-GL | CML(BCR-ABL1) | N | N |
| GSM236545 | #16-CP | CML(BCR-ABL1) | N | N |
| GSM236546 | #16-CP2 | CML(BCR-ABL1) | N | N |
| GSM236550 | #17-AP | CML(BCR-ABL1) | 5′amp->T | 3′amp->T |
| GSM236549 | #17-CP | CML(BCR-ABL1) | N | N |
| GSM236551 | #18-BC | CML(BCR-ABL1) | N | N |
| GSM236553 | #19-BC | CML(BCR-ABL1) | 5′amp->T | 3′amp->T |
| GSM236554 | #19-BC-GL | CML(BCR-ABL1) | N | N |
| GSM236552 | #19-CP | CML(BCR-ABL1) | N | N |
| GSM236511 | #1-BC | CML(BCR-ABL1) | N | N |
| GSM236510 | #1-CP | CML(BCR-ABL1) | N | N |
| GSM236556 | #20-AP | CML(BCR-ABL1) | N | N |
| GSM236557 | #20-BC | CML(BCR-ABL1) | N | N |
| GSM236555 | #20-CP | CML(BCR-ABL1) | N | N |
| GSM236558 | #21-CP | CML(BCR-ABL1) | N | N |
| GSM236559 | #21-CP2 | CML(BCR-ABL1) | N | N |
| GSM236561 | #22-BC | CML(BCR-ABL1) | 5′amp->T; 3′del->IGLL1 | 5′del->CDK9; 3′amp->T |
| GSM236562 | #22-BC-GL | CML(BCR-ABL1) | N | N |
| GSM236560 | #22-CP | CML(BCR-ABL1) | 3′del->IGLL1 | 5′del->CDK9 |
| GSM236564 | #23-BC | CML(BCR-ABL1) | N | N |
| GSM236565 | #23-BC-GL | CML(BCR-ABL1) | N | N |
| GSM236563 | #23-CP | CML(BCR-ABL1) | N | N |
| GSM236512 | #2-CP | CML(BCR-ABL1) | N | N |
| GSM236513 | #2-CP2 | CML(BCR-ABL1) | N | N |
| GSM236514 | #3-AP | CML(BCR-ABL1) | N | N |
| GSM236515 | #3-BC | CML(BCR-ABL1) | N | 3′amp->T |
| GSM236518 | #4-BC | CML(BCR-ABL1) | N | N |
| GSM236516 | #4-CP | CML(BCR-ABL1) | N | N |
| GSM236517 | #4-Rem | CML(BCR-ABL1) | N | N |
| GSM236521 | #5-BC | CML(BCR-ABL1) | N | N |
| GSM236520 | #5-BC-GL | CML(BCR-ABL1) | N | N |
| GSM236519 | #5-Rem | CML(BCR-ABL1) | N | N |
| GSM236524 | #6-BC | CML(BCR-ABL1) | 5′amp->T | 3′amp?->T |
| GSM236523 | #6-BC-GL | CML(BCR-ABL1) | N | N |
| GSM236522 | #6-CP | CML(BCR-ABL1) | N | N |
| GSM236526 | #7-BC | CML(BCR-ABL1) | N | N |
| GSM236525 | #7-CP | CML(BCR-ABL1) | N | N |
| GSM236528 | #8-AP | CML(BCR-ABL1) | N | N |
| GSM236527 | #8-CP | CML(BCR-ABL1) | N | N |
| GSM236529 | #9-BC | CML(BCR-ABL1) | N | N |
| GSM236530 | #9-BC-GL | CML(BCR-ABL1) | N | N |
| GSM235579 | #1 | E2A-PBX1 | 5′del->T | 3′amp->T |
| GSM235776 | #10 | E2A-PBX1 | 5′del->T | 3′amp->T |
| GSM235789 | #11 | E2A-PBX1 | 5′del?->T | 3′amp->T |
| GSM235804 | #12 | E2A-PBX1 | 5′del->T | 3′amp->T |
| GSM235813 | #13 | E2A-PBX1 | 5′del->T | 3′amp->T |
| GSM235820 | #14 | E2A-PBX1 | 5′del->T | 3′amp->T |
| GSM235854 | #15 | E2A-PBX1 | 5′del->T | 3′amp->T |
| GSM235859 | #16 | E2A-PBX1 | 5′del->T | 3′amp->T |
| GSM235861 | #17 | E2A-PBX1 | 5′del->T | 3′amp->T |
| GSM235602 | #2 | E2A-PBX1 | 5′del->T | 3′amp->T |
| GSM235620 | #3 | E2A-PBX1 | 5′del->T | 3′amp->T |
| GSM235625 | #4 | E2A-PBX1 | 5′del?->T | 3′amp->T |
| GSM235632 | #5 | E2A-PBX1 | 5′del?->T | 3′amp->T |
| GSM235641 | #6 | E2A-PBX1 | 5′del->T | 3′amp->T |
| GSM235650 | #7 | E2A-PBX1 | 5′del->T | 3′amp->T |
| GSM235668 | #8 | E2A-PBX1 | N | N |
| GSM235701 | #9 | E2A-PBX1 | 5′del?->T | 3′amp->T |
| GSM235670 | #10 | PAX5-ETV6 | PAX5:3′del->T | ETV6:5′del->T |
| GSM235611 | #3 | PAX5-ZNF521 | PAX5:3′del->T | ZNF521:5′del->T |
| GSM235631 | #1 | MLL- | N | N |
| GSM235846 | #10 | MLL- | N | N |
| GSM235866 | #11 | MLL- | N | N |
| GSM235563 | #12 | MLL- | N | N |
| GSM235578 | #13 | MLL- | N | N |
| GSM235627 | #15 | MLL- | N | N |
| GSM235673 | #16 | MLL- | N | MLLT3 5′del->C |
| GSM235712 | #17 | MLL- | N | N |
| GSM235742 | #18 | MLL- | N | N |
| GSM235746 | #19 | MLL- | N | N |
| GSM235633 | #2 | MLL- | 3′del->HYOU1 | APF1 int del |
| GSM235818 | #20 | MLL- | 3′del->BCL9L | N |
| GSM235847 | #21 | MLL- | N | N |
| GSM235855 | #22 | MLL- | N | N |
| GSM235869 | #23 | MLL- | N | N |
| GSM235652 | #3 | MLL- | 3′del->CBL | N |
| GSM235662 | #4 | MLL- | N | N |
| GSM235768 | #5 | MLL- | N | N |
| GSM235780 | #6 | MLL- | N | N |
| GSM235851 | #7 | MLL- | N | N |
| GSM235834 | #8 | MLL- | N | APF1 int del |
| GSM235837 | #9 | MLL- | N | N |
| GSM235828 | #14 | PAX5-FOXP1 | PAX5:3′del->PTPRD | FOXP1:n |
| GSM235561 | #1 | PAX5-ETV6 | PAX5:3′del->T | ETV6:5′del->T |
| GSM235566 | #1 | TEL-AML1 | 3′del->3′region | 5′del->5′region |
| GSM235601 | #10 | TEL-AML1 | N | N |
| GSM235603 | #11 | TEL-AML1 | int del | N |
| GSM235605 | #12 | TEL-AML1 | N | N |
| GSM235616 | #13 | TEL-AML1 | N | N |
| GSM235634 | #14 | TEL-AML1 | N | N |
| GSM235642 | #15 | TEL-AML1 | N | N |
| GSM235653 | #16 | TEL-AML1 | N | N |
| GSM235654 | #17 | TEL-AML1 | N | N |
| GSM235658 | #18 | TEL-AML1 | N | N |
| GSM235661 | #19 | TEL-AML1 | N | N |
| GSM235567 | #2 | TEL-AML1 | N | N |
| GSM235672 | #20 | TEL-AML1 | 3′del->chr12:028840592 | 3′amp->JAM2 |
| GSM235674 | #21 | TEL-AML1 | int del | N |
| GSM235684 | #22 | TEL-AML1 | 3′del->3′region | N |
| GSM235685 | #23 | TEL-AML1 | N | N |
| GSM235688 | #24 | TEL-AML1 | int del | N |
| GSM235696 | #25 | TEL-AML1 | N | N |
| GSM235699 | #26 | TEL-AML1 | int del | N |
| GSM235704 | #27 | TEL-AML1 | N | N |
| GSM235705 | #28 | TEL-AML1 | N | N |
| GSM235708 | #29 | TEL-AML1 | N | N |
| GSM235570 | #3 | TEL-AML1 | N | 5′del->TCC3 |
| GSM235754 | #30 | TEL-AML1 | N | 5′del->5′region |
| GSM235760 | #31 | TEL-AML1 | N | N |
| GSM235761 | #32 | TEL-AML1 | N | N |
| GSM235762 | #33 | TEL-AML1 | N | N |
| GSM235764 | #34 | TEL-AML1 | 3′del->chr12:017224159 | N |
| GSM235770 | #35 | TEL-AML1 | N | N |
| [symbol] | [symbol] | [symbol] | [symbol] | [symbol] |
| GSM235774 | #37 | TEL-AML1 | N | N |
| GSM235779 | #38 | TEL-AML1 | 3′del->C | N |
| GSM235788 | #39 | TEL-AML1 | 3′del->CDKN1B | N |
| GSM235571 | #4 | TEL-AML1 | N | N |
| GSM235794 | #40 | TEL-AML1 | N | N |
| GSM235800 | #41 | TEL-AML1 | 3′del->LRP6 | N |
| GSM235816 | #42 | TEL-AML1 | N | N |
| GSM235821 | #43 | TEL-AML1 | N | N |
| GSM235827 | #44 | TEL-AML1 | N | N |
| GSM235835 | #45 | TEL-AML1 | 3′del->BICD1 | 3′amp->T |
| GSM235857 | #46 | TEL-AML1 | N | N |
| GSM235862 | #47 | TEL-AML1 | N | N |
| GSM235845 | #48 | TEL-AML1 | N | int del |
| GSM235576 | #5 | TEL-AML1 | N | N |
| GSM235581 | #6 | TEL-AML1 | N | N |
| GSM235582 | #7 | TEL-AML1 | int del | N |
| GSM235585 | #8 | TEL-AML1 | N | N |
| GSM235597 | #9 | TEL-AML1 | N | N |

Note:
“N”, no change;
“amp”, amplification;
“del”, deletion;
“T”, telomere;
“C”, centromere;
“->” denotes the other end of the segmental deletion or amplification not generating the fusion.
The case contradicting the fusion breakpoint principle was marked with bold and italic.

TABLE 8
The summary of the genomic imbalances associated with gene fusions identified from
the 36 leukemia cell lines.

| GEO | | | Copy number abberation | Copy number abberation at |
|---|---|---|---|---|
| accession | Sample ID | Fusion | at 5′ gene locus | 3′ gene locus |
| GSM236815 | #BV173 | BCR-ABL1 | 5′amp->IGL@ | 5′del->C |
| GSM236820 | #K-562 | BCR-ABL1 | 5′amp->T | 3′amp->NUP214 |
| GSM236836 | #OP1 | BCR-ABL1 | N | N |
| GSM236840 | #SD1 | BCR-ABL1 | N | 5′del->5′region |
| GSM236842 | #SUPB-15 | BCR-ABL1 | N | N |
| GSM236843 | #THP-1 | BCR-ABL1 | N | N |
| GSM236844 | #TOM-1 | BCR-ABL1 | N | N |
| GSM236824 | #ME-1 | CBFB-MYH11 | N | N |
| GSM236846 | #UOCB1 | E2A-HLF | N | N |
| GSM236814 | #AT1 | ETV6-RUNX1 | N | N |
| GSM236823 | #KG-1 | FGFR1OP2-FGFR1 | 5′amp->chr12:025768792; | 3′amp->chr8:036399366 |
| | | | 3′del->AMN1 | |
| GSM236812 | #380 | IGH-BCL2 | int del | N |
| GSM236839 | #RS4_11 | MLL-AF4 | N | N |
| GSM236832 | #MV4-11 | MLL-AF4(AFF1) | N | N |
| GSM236827 | #ML-2 | MLL-AF6(MLLT4) | 3′del->T | 5′del->ESR1 |
| GSM236835 | #NOMO-1 | MLL-AF9 | N | 5′del->chr9:032018982 |
| GSM236843 | #THP-1 | MLL-AF9 | 3′amp->T | 3′amp->T |
| GSM236831 | #Mono-mac-6 | MLL-AF9(MLLT3) | N | N |
| GSM236812 | #380 | MYC-IGH | N | int del |
| GSM236845 | #U-937 | PICALM-AF10(MLLT10) | N | N |
| GSM236834 | #NB4 | PML-RARA | N | N |
| GSM236821 | #Kasumi-1 | RUNX1-RUNX1T1 | N | N |
| GSM236841 | #SKNO-1 | RUNX1-RUNX1T1 | N | N |
| GSM236816 | #CCRF-CEM | SIL(STIL)-SCL(TAL1) | N | N |
| GSM236813 | #697 | TCF3-PBX1 | N | 3′amp->T |
| GSM236822 | #Kasumi-2 | TCF3-PBX1 | 3′del->T | 3′amp->T |
| GSM236826 | #MHH-CALL-3 | TCF3-PBX1 | 3′del->T | 3′amp->T |
| GSM236838 | #Reh | TEL-AML1 | whole gene del | 5′amp->T |

Note:
“N”, no change;
“amp”, amplification;
“del”, deletion;
“T”, telomere;
“C”, centromere;
“->” denotes the other end of the segmental amplification or deletion not generating the fusion.

TABLE 9
Analysis and curation results of the public array CGH/array SNP/tiling CGH data
with gene fusions associated from publications.

| GEO | | | | | DNA | Copy number | Copy number |
|---|---|---|---|---|---|---|---|
| accession/ | | GSM | | | Breakpoint | abberation at 5′ | abberation at 3′ |
| Puluned ID | Cancer | accession | Sample ID | Fusion | Pattern ID | gene locus | gene locus |
| GSE9611 | ALL | GSM243107 | #1 | BCR-ABL1 | — | N | N |
| GSE9611 | ALL | GSM243108 | #2 | BCR-ABL1 | #1 | 5′amp->T; | 5′del->CCBL1; |
| | | | | | | 3′del->LOC51233 | 3′amp->OBP2B |
| GSE9611 | ALL | GSM243109 | #3 | BCR-ABL1 | #2 | 5′amp->T | N |
| GSE9611 | ALL | GSM243110 | #4 | BCR-ABL1 | — | N | N |
| GSE9611 | ALL | GSM243111 | #5 | BCR-ABL1 | #2 | 5′amp->T | N |
| GSE9611 | ALL | GSM243112 | #6 | BCR-ABL1 | #3 | 5′amp->T | int del |
| GSE9611 | ALL | GSM243113 | #7 | BCR-ABL1 | — | N | N |
| GSE9611 | ALL | GSM243114 | #8 | BCR-ABL1 | #4 | 5′amp->T | 5′amp->C |
| GSE9611 | ALL | GSM243115 | #9 | BCR-ABL1 | — | N | N |
| GSE9611 | ALL | GSM243116 | #10 | BCR-ABL1 | — | N | N |
| GSE9611 | ALL | GSM243119 | #13 | IGH-MYC | #5 | 3′del->T | N |
| GSE9611 | ALL | GSM243120 | #14 | IGH-MYC | — | N | N |
| GSE7255 | ALL | GSM174868 | 9348(#1) | MLL-AF5 | #6 | 3′del->CBL | N |
| GSE7255 | ALL | GSM174860 | 9225(#2) | MLL-AF4 | #7 | 3′del->DDX6 | 5′del->LOC442777 |
| GSE7255 | ALL | GSM174830 | 9256(#3) | ETV6-RUNX1 | — | N | N |
| GSE7255 | ALL | GSM174846 | 9418(#4) | ETV6-RUNX1 | #8 | 5′amp->T | N |
| GSE7255 | ALL | GSM174851 | 9357(#5) | ETV6-RUNX1 | — | N | N |
| GSE7255 | ALL | GSM174852 | 9393(#6) | E2A-PBX1 | #9 | N | 3′amp->T |
| GSE8918 | NHL | GSM226057 | FL#1 | IgH-BCL2(90%) | — | N | N |
| GSE8918 | NHL | GSM226058 | FL#2 | IgH-BCL2(90%) | — | N | N |
| GSE8918 | NHL | GSM226059 | FL#3 | IgH-BCL2(90%) | — | N | N |
| GSE8918 | NHL | GSM226060 | FL#4 | IgH-BCL2(90%) | — | N | N |
| GSE8918 | NHL | GSM226061 | FL#5 | IgH-BCL2(90%) | — | N | N |
| GSE8918 | NHL | GSM226062 | FL#6 | IgH-BCL2(90%) | — | N | N |
| GSE8918 | NHL | GSM226063 | FL#7 | IgH-BCL2(90%) | — | N | N |
| GSE8918 | NHL | GSM226064 | FL#8 | IgH-BCL2(90%) | — | N | N |
| GSE8918 | NHL | GSM226065 | FL#9 | IgH-BCL2(90%) | — | N | N |
| GSE8918 | NHL | GSM226066 | FL#10 | IgH-BCL2(90%) | — | N | N |
| GSE8918 | NHL | GSM226067 | FL#11 | IgH-BCL2(90%) | — | N | N |
| GSE8918 | NHL | GSM226068 | FL#12 | IgH-BCL2(90%) | #12 | N | 3′amp->T |
| GSE8918 | NHL | GSM226069 | FL#13 | IgH-BCL2(90%) | #13 | 3′del?->T | N |
| GSE8918 | NHL | GSM226070 | FL#14 | IgH-BCL2(90%) | — | N | N |
| GSE8918 | NHL | GSM226071 | FL#15 | IgH-BCL2(90%) | — | N | N |
| GSE8918 | NHL | GSM226088 | MCL#31 | IgH-CCND1(95%) | #14 | 3′del?->T | 3′amp->CENTD2 |
| GSE8918 | NHL | GSM226089 | MCL#32 | IgH-CCND1(95%) | — | N | N |
| GSE8918 | NHL | GSM226090 | MCL#33 | IgH-CCND1(95%) | #15 | 5′amp?->IAG2 | N |
| GSE8918 | NHL | GSM226091 | MCL#34 | IgH-CCND1(95%) | — | N | N |
| GSE8918 | NHL | GSM226092 | MCL#35 | IgH-CCND1(95%) | — | N | N |
| GSE8918 | NHL | GSM226093 | MCL#36 | IgH-CCND1(95%) | #16 | 3′del?->T | N |
| GSE8918 | NHL | GSM226094 | MCL#37 | IgH-CCND1(95%) | — | N | N |
| GSE8918 | NHL | GSM226095 | MCL#38 | IgH-CCND1(95%) | #17 | N | 3′amp->UVRAG |
| GSE8918 | NHL | GSM226096 | MCL#39 | IgH-CCND1(95%) | — | N | N |
| GSE8918 | NHL | GSM226097 | MCL#40 | IgH-CCND1(95%) | — | N | N |
| GSE8918 | NHL | GSM226098 | MCL#41 | IgH-CCND1(95%) | — | N | N |
| GSE8918 | NHL | GSM226099 | MCL#42 | IgH-CCND1(95%) | — | N | N |
| GSE8918 | NHL | GSM226100 | MCL#43 | IgH-CCND1(95%) | — | N | N |
| GSE8918 | NHL | GSM226101 | MCL#44 | IgH-CCND1(95%) | — | N | N |
| GSE8918 | NHL | GSM226111 | LPL#54 | IgH-PAX5(50%) | — | N | N |
| GSE8918 | NHL | GSM226112 | LPL#55 | IgH-PAX5(50%) | — | N | N |
| GSE8918 | NHL | GSM226113 | LPL#56 | IgH-PAX5(50%) | — | N | N |
| GSE8918 | NHL | GSM226114 | LPL#57 | IgH-PAX5(50%) | — | N | N |
| GSE8918 | NHL | GSM226115 | LPL#58 | IgH-PAX5(50%) | — | N | N |
| GSE8918 | NHL | GSM226116 | LPL#59 | IgH-PAX5(50%) | — | N | N |
| GSE8918 | NHL | GSM226117 | LPL#60 | IgH-PAX5(50%) | — | N | N |
| GSE8918 | NHL | GSM226118 | LPL#61 | IgH-PAX5(50%) | — | N | N |
| GSE8918 | NHL | GSM226119 | LPL#62 | IgH-PAX5(50%) | — | N | N |
| GSE8918 | NHL | GSM226120 | LPL#63 | IgH-PAX5(50%) | — | N | N |
| GSE8918 | NHL | GSM226136 | MALT#79 | BIRC3-MALT1(30%)* | — | N | N |
| GSE8918 | NHL | GSM226137 | MALT#80 | BIRC3-MALT1(30%)* | — | N | N |
| GSE8918 | NHL | GSM226138 | MALT#81 | BIRC3-MALT1(30%)* | — | N | N |
| GSE8918 | NHL | GSM226139 | MALT#82 | BIRC3-MALT1(30%)* | — | N | N |
| GSE8918 | NHL | GSM226140 | MALT#83 | BIRC3-MALT1(30%)* | — | N | N |
| GSE8918 | NHL | GSM226141 | MALT#84 | BIRC3-MALT1(30%)* | — | N | N |
| GSE8918 | NHL | GSM226142 | MALT#85 | BIRC3-MALT1(30%)* | — | N | N |
| GSE8918 | NHL | GSM226143 | MALT#86 | BIRC3-MALT1(30%)* | — | N | N |
| GSE8918 | NHL | GSM226144 | MALT#87 | BIRC3-MALT1(30%)* | — | N | N |
| GSE8398 | EWS | GSM207892 | #1 | EWSR1-FLI1 | — | N | N |
| GSE8398 | EWS | GSM207893 | #2 | EWSR1-FLI1 | — | N | N |
| GSE8398 | EWS | GSM207894 | #3 | EWSR1-FLI1 | — | N | N |
| GSE8398 | EWS | GSM207895 | #4 | EWSR1-FLI1 | #19 | N | 5′del->TMEM135 |
| GSE8398 | EWS | GSM207896 | #5 | EWSR1-FLI1 | #20 | 5′amp->T | 3′amp->T |
| GSE8398 | EWS | GSM207897 | #6 | EWSR1-FLI1 | — | N | N |
| GSE8398 | EWS | GSM207898 | #7 | EWSR1-FLI1 | — | N | N |
| GSE8398 | EWS | GSM207899 | #8 | EWSR1-FLI1 | — | N | N |
| GSE8398 | EWS | GSM207900 | #9 | EWSR1-FLI1 | — | N | N |
| GSE8398 | EWS | GSM207901 | #10 | EWSR1-FLI1 | — | N | N |
| GSE8398 | EWS | GSM207902 | #12 | EWSR1-FLI1 | — | N | N |
| GSE8398 | EWS | GSM207903 | #13 | EWSR1-FLI1 | — | N | N |
| GSE8398 | EWS | GSM207904 | #14 | EWSR1-FLI1 | — | N | N |
| GSE8398 | EWS | GSM207905 | #15 | EWSR1-FLI1 | — | N | N |
| GSE8398 | EWS | GSM207906 | #16 | EWSR1-FLI1 | — | N | N |
| GSE8398 | EWS | GSM207907 | #17 | EWSR1-FLI1 | — | N | N |
| GSE8398 | EWS | GSM207908 | #18 | EWSR1-FLI1 | — | N | N |
| GSE8398 | EWS | GSM207909 | #19 | EWSR1-FLI1 | — | N | N |
| GSE8398 | EWS | GSM207910 | #20 | EWSR1-FLI1 | — | N | N |
| GSE8398 | EWS | GSM207911 | #21 | EWSR1-FLI1 | — | N | N |
| GSE8398 | EWS | GSM207912 | #22 | EWSR1-FLI1 | — | N | N |
| GSE8398 | EWS | GSM207913 | #23 | EWSR1-FLI1 | — | N | N |
| GSE8398 | EWS | GSM207914 | #24 | EWSR1-FLI1 | — | N | N |
| GSE8398 | EWS | GSM207915 | #25 | EWSR1-FLI1 | — | N | N |
| GSE8398 | EWS | GSM207916 | #26 | EWSR1-FLI1 | — | N | N |
| 16193090 | B-NHL | NA | 581/90(#1) | IGH-BCL2 | #18 | ? | 3′amp |
| 16193090 | B-NHL | NA | 364/86(#2) | IGH-BCL2 | #18 | ? | 3′amp |
| 16193090 | B-NHL | NA | 436/91(#3) | IGH-BCL2 | #18 | ? | 3′amp |
| 16193090 | B-NHL | NA | 176/88(#4) | IGH-BCL2 | #18 | ? | 3′amp |
| 16193090 | B-NHL | NA | 472/90(#5) | IGH-BCL2 | #18 | ? | 3′amp |
| 16193090 | B-NHL | NA | 287/88(#6) | IGH-BCL2 | #18 | ? | 3′amp |
| 16193090 | B-NHL | NA | 21/87(#7) | IGH-BCL2 | #18 | ? | 3′amp |
| 16193090 | B-NHL | NA | 190/92(#8) | IGH-BCL2 | — | ? | N |
| 16193090 | B-NHL | NA | 377/83(#9) | IGH-BCL2 | — | ? | N |
| 16193090 | B-NHL | NA | 311/89(#10) | IGH-BCL2 | — | ? | N |
| 16193090 | B-NHL | NA | 41/88(#11) | IGH-BCL2 | — | ? | N |
| 16193090 | B-NHL | NA | 64/89(#12) | IGH-BCL2 | — | ? | N |
| 16193090 | B-NHL | NA | 34/90(#13) | IGH-BCL2 | — | ? | N |
| 16193090 | B-NHL | NA | 381/88(#14) | IGH-BCL2 | — | ? | N |
| 16193090 | B-NHL | NA | 140/90(#15) | IGH-BCL2 | — | ? | N |
| 16193090 | B-NHL | NA | 345/87(#16) | IGH-BCL2 | — | ? | N |
| 16193090 | B-NHL | NA | 140/90(#17) | IGH-BCL2 | — | ? | N |
| 16193090 | B-NHL | NA | 345/87(#18) | IGH-BCL2 | — | ? | N |
| 15361874 | T-ALL | NA | #1 | NUP214-ABL1 | #10 | 5′amp->ABL1 | 3′amp->NUP214 |
| 15361874 | T-ALL | NA | #2 | NUP214-ABL1 | #10 | 5′amp->ABL1 | 3′amp->NUP214 |
| 15361874 | T-ALL | NA | #3 | NUP214-ABL1 | #10 | 5′amp->ABL1 | 3′amp->NUP214 |
| 15361874 | T-ALL | NA | #4 | NUP214-ABL1 | #10 | 5′amp->ABL1 | 3′amp->NUP214 |
| 15361874 | T-ALL | NA | #5 | NUP214-ABL1 | #10 | 5′amp->ABL1 | 3′amp->NUP214 |
| 15361874 | T-ALL | NA | #6 | NUP214-ABL1 | #10 | 5′amp->ABL1 | 3′amp->NUP214 |
| 10681437 | AML | NA | #1 | MLL-LARG | #11 | 3′del->LARG | 5′del->MLL |
| GSE3930 | DFSP | GSM89915 | STT154(#1) | COLIA1-PDGFB | #21 | 5′amp | 3′amp |
| GSE3930 | DFSP | GSM89909 | STT154(#2) | COL1A1-PDGFB | #21 | 5′amp | 3′amp |
| GSE3930 | DFSP | GSM89916 | STT491(#3) | COL1A1-PDGFB | #21 | 5′amp | 3′amp |
| GSE3930 | DFSP | GSM89911 | STT491(#4) | COL1A1-PDGFB | #21 | 5′amp | 3′amp |
| GSE3930 | DFSP | GSM89919 | STT1984(#5) | COL1A1-PDGFB | #21 | 5′amp | 3′amp |
| GSE3930 | DFSP | GSM89904 | STT1984(#6) | COL1A1-PDGFB | #21 | 5′amp | 3′amp |
| GSE3930 | DFSP | GSM89931 | STT1971(#7) | COL1A1-PDGFB | #22 | 5′amp | N |
| 17124411 | DFSP | NA | 7 cases | COL1A1-PDGFB | #21 | 5′amp in 5 cases | 3′amp in 3 cases |
| 11244503 | ASPS | NA | 12 cases | ASP8CR1-TPE3 | #23 | 3′del in 9 cases | 3′amp in 9 cases |
| 18974108 | AST | NA | 29 cases | KLAA1549-BRAF | #24 | 5′amp in 29 cases | 3′amp in 29 cases |
| 18059337 | SPA | NA | 11 cases | FGFR1-PLAG1 | #25 | 5′amp in 10 cases | 3′amp in 10 cases |
| 17654723 | CaP | NA | 106 cases | TMPRSS2-ERG | #26 | 3′del in 54 cases | 5′amp in 54 cases |
| 16951139 | | | | | | | |

ASPS, Alveolar soft part sarcoma;
DFSP, Dermatofibrosarcoma Protruberans;
AML, Acute Myelogenous Leukemia;
ALL, Acute Lymphoblastic Lymphoma;
EWS, Ewings' sarcoma;
NHL, non-hodgekin lymphoma;
AST, Brain Astrocytoma;
LUG, Lung Carcinoma;
CaP, Prostate Adenocarcinoma;
SPA, Salivary Pleomorphic Adenoma.
*In MALT, besides BIRC3-MALT1 reported in 30% cases, there were also IgH-MALT1 reported in 15-20% cases, IgH-FOXP1 in 10% cases and IgH-BCL10 in 5% cases.

TABLE 10
Curation of the experimental data showing the genomic abberations for all intra-
chromosome gene fusions from the Mitelman database.

| | | Genomic | | | | | |
|---|---|---|---|---|---|---|---|
| | | Distance | Predicted | | Puluned ID of | | |
| | Cancer | between fusion | genomic | Total No. of | informative | Experimental | |
| Gene Fusion | Type | partners (Lb) | imbalance | reports | reports | Methods | Curation Results* |
| MLL/ARHGEF17 | AML | 45055 | amp | 1 | — | — | no information |
| TMP3/TPR | THY | 32118 | amp | 1 | — | — | no information |
| RPN1/EVI1 | AML | 40433 | amp | 2 | — | — | no information |
| NUP214/ABL1 | ALL | 237 | amp | 1 | 15361874 | Tiling CGH | interstitial amplicon |
| PRKAR1A/RARA | APL | 28252 | amp | 1 | 17712046 | FISH | no unbalance info |
| TCEA1/PLAG1 | SAL | 2138 | amp | 2 | 16736500 | FISH | no unbalance info |
| MLL/DCPS | AML | 7778 | del | 1 | — | — | no information |
| TFRC/BCL6 | B-LYM | 8315 | del | 1 | — | — | no information |
| HAS2/PLAG1 | LPB | 65408 | del | 2 | 10987300 | FISH | bac not locatable |
| TMPR882/ERG | CsP | 2803 | del | 10 | 16951139 | aCGH | interstitial deletion |
| HNRPA2B1/ETV1 | CaP | 12203 | del | 1 | 17671502 | FISH | del 3′ HNRPA2B1; del |
| | | | | | | | 5′ETV1 |
| MLL/CBL | AML | 681 | del | 1 | 12696071 | FISH | del 3′MLL |
| MLL/ARHGEF12 | AML | 1812 | del | 2 | 10681437 | inference | del 3′MLL; del 5′LARG |
| FIP1L1/PDGFRA | CEL | 770 | del | 4 | 12660384, | FISH | del internal BAC a$$ |
| | | | | | 14973504 | | CHIC2 locus |
| SET/NUP214 | AML | 2492 | del | 2 | 17296573 | aCGH | interstitial deletion |
| STIL/TAL1 | ALL | 20 | del | 2 | 8459224 | citation | interstitial deletion |
| GOPC/ROSI | GBM | 134 | del | 1 | 12661006 | sequencing | interstitial deletion |
| MLL/TIRAP | AML | 7757 | del | 1 | 15626757 | RT-PCR | no reciprocal fusion |
| RET/NCOA4 | THY | 8297 | del | 4 | — | — | no information |
| LPP/BCL6 | B-LYM | 467 | inv | 1 | — | — | no information |
| MLL/BCL9L | ALL | 371 | inv | 1 | — | — | no information |
| MLL/MAML2 | AML | 22096 | inv | 1 | — | — | no information |
| BCL11B/TRD@ | ALL | 76700 | inv | 1 | 15558700 | FISH | balanced inversion |
| TRA@/TCLLA | NLD | 73155 | inv | 1 | 7662982 | FISH | dnp pf 5′& 3′TCL1A |
| RET/CCDC6 | THY | 18274 | inv | 5 | 1542652 | Southern blot | reciprocal fusion |
| EML4/ALK | LUG | 12252 | inv | 3 | 18083107 | FISH | del 5′ ALK |
| EWSR1/PATZ1 | SAR | 2025 | inv | 1 | 10949935 | RT-PCR | no reciprocal fusion |
| MLL/PICALM | AML; ALL | 32355 | inv | 2 | 12461747 | RT-PCR | no reciprocal fusion |
| CHCHD7/PLAGI | SAL | 0 | inv | 1 | 16736500 | FISH | no unbalanced info |
| AKAP9/BRAF | THY | 48503 | inv | 1 | 15630448 | RT-PCR | reciprocal fusion |
| TPM3/NTEK1 | THY | 2621 | inv | 2 | 7590742 | RT-PCR | reciprocal fusion |
| AFF1/ELF2 | ALL | 51917 | inv | 1 | 17410185 | RT-PCR | three way balanced |
| DSCAML1/MLL | ALL | 639 | inv | 1 | 17410185 | RT-PCR | three way balanced |
| FXYD6/MLL | ALL | 560 | inv | 1 | 17410185 | RT-PCR | three way balanced |

“No.”: the number of publications reporting the gene fusions.
Note:
Amp, segmental amplification,
Del: interstitial deletion,
inv: inversion.
APL, Acute promyelocytic leukemia;
AUL, Acute undifferentiated leukemia;
CEL, Chronic eosinophilic leukemia;
MYE, Myeloproliferative disease;
SAR, Sarcoma;
LPB, Lipoblastoma;
LYM, Lymphoma;
F-LYM, Follicular Lymphoma;
T-LYM, T-cell Lymphoma;
B-LYM, B-cell Lymphoma,
M-LYM, Mantle Cell Lymphoma;
NLD, Nonneoplastic lymphatic disorder;
LPL, Lymphoplasmacytic Lymphoma;
THY, Thyroid Adenocarcinoma.
*Amplifications/deletions fit to the prediction from the inferred principle as well as translocations without genomic imbalances are considered as following the inferred principle.

TABLE 11
The summary of FISH findings for unbalanced ETS gene
fusions in 171 prostate cancer cases (UM cohort)

| Case (n) | 3′ ETS Gene | FISH finding | 5′ fusion partner | FISH finding |
|---|---|---|---|---|
| 44 | ERG | 5′deletion | TMPRSS2 | 3′ deletion |
| 4 | ERG | split | TMPRSS2 | 3′ deletion |
| 3 | ERG | 5′ deletion | TMPRSS2 | split |
| 5 | ERG | 5′deletion; | TMPRSS2 | 3′ deletion; |
| | | 3′duplication | | 5′ duplication |
| 1 | ETV1 | split | HNRPA2B1 | 3′ deletion |
| 1 | ETV1 | 5′deletion | C15ORF21 | split |
| 1 | ETV4 | 5′deletion | TMPRSS2 | split |
| 1 | ETV4 | 5′deletion | CANT1 | 3′ deletion |

TABLE 12
The split-apart probes used for fluorescence in situ hybridization
detecting ETS gene rearrangements in prostate cancer.

| | Chromosome | | |
|---|---|---|---|
| Gene | band | 5′ region | 3′ region |
| ERG | 21q22.2 | RP11-95I21 | RP11-476D17 |
| ETV1 | 7p21.2 | RP11-703A4 | RP11-124L22 |
| ETV4 | 17q21.31 | RP11-436J4 | RP11-100E5 |
| ETV5 | 3q27.2 | RP11-379C23 | RP11-1144N13 |
| TMPRSS2 | 21q22.3 | RP11-35C4 | RP11-120C17 |
| SLC45A3 | 1q32.1 | RP11-1089F13 | RP11-1143H2 |
| C15ORF21 | 15q21.1 | RP11-474E1 | RP11-626F7 |
| HERV-K_22q11.23 | 22q11.23 | RP11-947A12 | RP11-61P17 |
| HNRPA2B1 | 7p15.2 | RP11-379M24 | RP11-11F13 |
| FLJ35294 | 17p13.1 | RP11-1099M24 | RP11-55C13 |
| CANT1 | 17q25.3 | RP11-52K16 | RP11-46K10 |
| KLK2 | 19q13.33 | CTC-771P3 | RP11-26P14 |
| DDX5 | 17q24.1 | RP11-81D7 | RP11-315N9 |

TABLE 13
Normal tissues from the “Both_Normal” dataset analyzed
using Oncomine database²⁰. The gene expression data of
NFE2 across the 40 normal tissue types indicated in this table
are displayed in FIG. 13.

| No. | Tissue Type | Sample Number (n) |
|---|---|---|
| 1 | Adipose | 10 |
| 2 | Adrenal Gland Cortex | 4 |
| 3 | Bone Marrow | 5 |
| 4 | Bronchus | 3 |
| 5 | Cervix | 4 |
| 6 | Colon Cecum | 3 |
| 7 | Coronary Artery | 3 |
| 8 | Dorsal Root Ganglia | 8 |
| 9 | Endometrium | 4 |
| 10 | Esophagus | 4 |
| 11 | Heart Atrium | 4 |
| 12 | Heart Ventricle | 3 |
| 13 | Kidney Cortex | 4 |
| 14 | Kidney Medulla | 4 |
| 15 | Liver | 4 |
| 16 | Lymph Nodes | 4 |
| 17 | Mammary Gland | 3 |
| 18 | Myometrium | 5 |
| 19 | Nipple Cross-Section | 4 |
| 20 | Nodose Nucleus | 8 |
| 21 | Oral Mucosa | 4 |
| 22 | Ovary | 4 |
| 23 | Pharyngeal Mucosa | 4 |
| 24 | Pituitary Gland | 8 |
| 25 | Prostate Gland | 3 |
| 26 | Salivary Gland | 4 |
| 27 | Saphenous Vein | 3 |
| 28 | Skeletal Muscle | 5 |
| 29 | Spleen | 4 |
| 30 | Stomach | 11 |
| 31 | Testes | 3 |
| 32 | Thyroid Gland | 4 |
| 33 | Tongue | 8 |
| 34 | Tonsil | 3 |
| 35 | Trachea | 3 |
| 36 | Trigeminal Ganglia | 8 |
| 37 | Urethra | 3 |
| 38 | Vagina | 4 |
| 39 | Vulva | 4 |
| 40 | Lung | 3 |

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

We claim:

1. A method for identifying lung cancer in a patient comprising:
(a) providing a sample from the patient; and
(b) detecting the presence or absence in the sample of a gene fusion having a 5′ portion from a transcriptional regulatory region of an R3HDM2 gene and a 3′ portion from a NFE2 gene,
wherein detecting the presence in the sample of the gene fusion identifies lung cancer in the patient.

2. The method of claim 1, wherein the transcriptional regulatory region of the R3HDM2 gene comprises a promoter region of the R3HDM2 gene.

3. The method of claim 1, wherein step (b) comprises detecting chromosomal rearrangements of genomic DNA having a 5′ DNA portion from the transcriptional regulatory region of the R3HDM2 gene and a 3′ DNA portion from the NFE2 gene.

4. The method of claim 1, wherein step (b) comprises detecting chimeric mRNA transcripts having a 5′ RNA portion transcribed from the transcriptional regulatory region of the R3HDM2 gene and a 3′ RNA portion transcribed from a NFE2 gene.

5. The method of claim 1, wherein the sample is selected from the group consisting of tissue, blood, plasma, serum and lung cells.

6. A composition comprising at least one of the following:
(a) an oligonucleotide probe comprising a sequence that hybridizes to a junction of a chimeric genomic DNA or chimeric mRNA in which a 5′ portion of the chimeric genomic DNA or chimeric mRNA is from a transcriptional regulatory region of an R3HDM2 gene and a 3′ portion of the chimeric genomic DNA or chimeric mRNA is from a NFE2 gene;
(b) a first oligonucleotide probe comprising a sequence that hybridizes to a 5′ portion of a chimeric genomic DNA or chimeric mRNA from a transcriptional regulatory region of an R3HDM2 gene and a second oligonucleotide probe comprising a sequence that hybridizes to a 3′ portion of the chimeric genomic DNA or chimeric mRNA from a NFE2 gene;
(c) a first amplification oligonucleotide comprising a sequence that hybridizes to a 5′ portion of a chimeric genomic DNA or chimeric mRNA from a transcriptional regulatory region of an R3HDM2 gene and a second amplification oligonucleotide comprising a sequence that hybridizes to a 3′ portion of the chimeric genomic DNA or chimeric mRNA from a NFE2 gene;
(d) an antibody to a chimeric protein having an animo-terminal portion encoded by the R3HDM2 gene and a carboxy-terminal portion encoded by a NFE2 gene; and
(e) an antibody to an overexpressed NFE2 gene.


SEQUENCE LISTING (14 sequences)

SEQ ID NO: 1 - DNA, 20 bp, Artificial Sequence
  Synthetic
actcatggaggctgagcatt

SEQ ID NO: 2 - DNA, 20 bp, Artificial Sequence
  Synthetic
agctcggtgatggacatgat

SEQ ID NO: 3 - DNA, 25 bp, Artificial Sequence
  Synthetic
agcagggtgacccctgatgttgccc

SEQ ID NO: 4 - DNA, 25 bp, Artificial Sequence
  Synthetic
actcccccaaactgttttcctggct

SEQ ID NO: 5 - DNA, 25 bp, Artificial Sequence
  Synthetic
agcagggtgacccctgatgttgccc

SEQ ID NO: 6 - DNA, 24 bp, Artificial Sequence
  Synthetic
tggtccaggttcccggaaagccca

SEQ ID NO: 7 - DNA, 24 bp, Artificial Sequence
  Synthetic
tggcccagtaggatgtccccgtgt

SEQ ID NO: 8 - DNA, 26 bp, Artificial Sequence
  Synthetic
gtggacagctgtatcaccctgttcct

SEQ ID NO: 9 - DNA, 26 bp, Artificial Sequence
  Synthetic
tccccagcagagcaggaacagggtga

SEQ ID NO: 10 - DNA, 26 bp, Artificial Sequence
  Synthetic
aaggtatggagctggggcttggggct

SEQ ID NO: 11 - DNA, 21 bp, Artificial Sequence
  Synthetic
ctgaatctcttgagctggagg

SEQ ID NO: 12 - DNA, 22 bp, Artificial Sequence
  Synthetic
gctggcaaggtatagttggagt

SEQ ID NO: 13 - DNA, 20 bp, Artificial Sequence
  Synthetic
tgcaccaccaactgcttagc

SEQ ID NO: 14 - DNA, 21 bp, Artificial Sequence
  Synthetic
ggcatggactgtggtcatgag